(19) United States
(12) Patent Application Publication
(10) Pub. No.: US 2023/0038233 A1
(43) Pub. Date: Feb. 9, 2023

(54) TREATMENT OF NEUROLOGICAL DISORDERS

(71) Applicant: IPSEN BIOPHARM LIMITED, Wrexham (GB)

(72) Inventors: Elena FONFRIA SUBIROS, Wrexham (GB); Agnieszka LEWANDOWSKA, Wrexham (GB)

(21) Appl. No.: 17/754,216

(22) PCT Filed: Sep. 30, 2020
(86) PCT No.: PCT/GB2020/052363

§ 371 Date: Mar. 28, 2022

(30) Foreign Application Priority Data
Sep. 30, 2019 (GB) 1914034.2

(51) Int. Cl. A61K 38/48; A61P 25/28

(52) U.S. Cl. CPC A61K 38/4893; A61P 25/28

(57) ABSTRACT

The present invention is directed to a polypeptide for use in promoting neuronal growth or neuronal repair to treat a neurological disorder in a subject, wherein the polypeptide comprises: a clostridial neurotoxin light chain (L-chain) or fragment thereof; and/or a fragment of a clostridial neurotoxin heavy chain (H-chain). Additional polypeptides for use in promoting neuronal growth or neuronal repair to treat a neurological disorder in a subject are also provided, as are corresponding methods and uses.

The present invention relates to the treatment of neurological disorders.

Neurological disorders include neuronal injuries, neurodegenerative disorders, sensory disorders, and autonomic disorders.

Neuronal injuries, such as spinal cord injuries (SCI), induce degeneration of injured axons preventing normal sensory, motor, and autonomic function. Recovery can occur by endogenous mechanisms such as regeneration of injured axons and the collateral sprouting of undamaged axons, resulting in the reinnervation of denervated targets. However, the regenerative capacity of the injured neurons (especially the spinal cord) is limited in adult mammals and patients can suffer various disabilities which greatly impact quality of life.

Conventional therapeutics for neuronal injuries include interleukin-6 (IL-6) and stem cell transplantation, however few are at a phase of development for use in the clinic. Therefore, there remains a need for a therapeutic for neuronal injuries capable of promoting neuronal growth or repair.

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies.

WO 2016/170501 A1 describes the use of catalytically active full-length BoNT/A (containing the L-chain and complete H-chain including the H_{N} and H_{C} domains) for the treatment of paralysis caused by spinal cord injury. WO 2016/170501 A1 teaches that each of the functional domains of BoNT/A are essential for the therapeutic effects observed, including the H-chain binding and translocation capabilities and the L-chain non-cytotoxic protease activity. As described above, full-length clostridial neurotoxins are extremely potent, necessitating adoption of specific safety procedures when handling the toxin. Moreover, spread of toxin away from the target tissue is believed to be responsible for undesirable side effects that in extreme cases may be life threatening. This can be a particular concern when using clostridial neurotoxin therapeutics (such as BoNT therapeutics) at high doses, concentrations and injection volumes. Adverse effects associated with this problem that have been reported for commercial BoNT/A therapeutics include asthenia, generalised muscle weakness, diplopia, ptosis, dysphagia, dysphonia, dysarthria, urinary incontinence, and breathing difficulties. Swallowing and breathing difficulties can be life threatening and there have been reported deaths related to the spread of toxin effects. Thus, there is a need for a safer therapeutic for promoting neuronal growth or repair.

Given their size, use of the full-length clostridial neurotoxins (˜150 kDa) or complete H-chains thereof (˜100 kDa) is associated with an increased risk of eliciting an immune response in a subject being treated with said polypeptide. Moreover, the presence of the entire H-chain (and in particular the H_{C} domain) results in polypeptide binding to clostridial neurotoxin target receptors, which may be associated with unwanted off-target effects in a subject administered said polypeptide.

The present invention overcomes one or more of the above-mentioned problems.

The present inventors have surprisingly found that a polypeptide comprising a clostridial neurotoxin L-chain and/or a fragment of a clostridial neurotoxin H-chain (e.g. the translocation domain (H_{N}) or the receptor binding domain (H_{C})) promotes neuronal growth or repair, and thus finds utility in treating neurological disorders. Advantageously, this allows for the use of non-toxic (or substantially non-toxic) fragments of clostridial neurotoxins, which given the smaller size (compared to the full-length H-chain or full-length clostridial neurotoxin), are less likely to provoke an immune response in a subject administered said fragments. Moreover, the non-toxic (or substantially non-toxic) fragments are less expensive and/or less complex to manufacture than full-length clostridial neurotoxins. Additionally, the non-toxic (or substantially non-toxic) fragments constitute a more well-defined therapeutic than the full-length clostridial toxins, and given the shorter length of the polypeptides there is a reduced probability of, for example, cysteine shuffling between domains.

Thus, in one aspect the invention provides a polypeptide for use in promoting neuronal growth or neuronal repair to treat a neurological disorder in a subject, wherein the polypeptide comprises:
a clostridial neurotoxin light chain (L-chain) or fragment thereof; and/or
a fragment of a clostridial neurotoxin heavy chain (H-chain).

In a related aspect there is provided a method for promoting neuronal growth or neuronal repair to treat a neurological disorder in a subject, the method comprising administering a polypeptide to the subject, wherein the polypeptide comprises:
a clostridial neurotoxin L-chain or fragment thereof; and/or
a fragment of a clostridial neurotoxin H-chain.

In another aspect there is provided use of a polypeptide in the manufacture of a medicament for promoting neuronal growth or neuronal repair to treat a neurological disorder in a subject, wherein the polypeptide comprises:
a clostridial neurotoxin L-chain or fragment thereof; and/or
a fragment of a clostridial neurotoxin H-chain.

In one aspect the invention provides a polypeptide for use in treating a neurological disorder in a subject, wherein the polypeptide comprises:
a clostridial neurotoxin light chain (L-chain) or fragment thereof; and/or
a fragment of a clostridial neurotoxin heavy chain (H-chain).

In a related aspect there is provided a method for treating a neurological disorder in a subject, the method comprising administering a polypeptide to the subject, wherein the polypeptide comprises:
a clostridial neurotoxin L-chain or fragment thereof; and/or
a fragment of a clostridial neurotoxin H-chain.

In another aspect there is provided use of a polypeptide in the manufacture of a medicament for treating a neurological disorder in a subject, wherein the polypeptide comprises:
a clostridial neurotoxin L-chain or fragment thereof; and/or
a fragment of a clostridial neurotoxin H-chain.

In one embodiment a polypeptide of the invention comprises a clostridial neurotoxin L-chain. It is preferred that the L-chain is catalytically inactive.

Thus, in one aspect, the invention provides a polypeptide for use in promoting neuronal growth or neuronal repair to treat a neurological disorder in a subject, wherein the polypeptide comprises a catalytically inactive clostridial neurotoxin L-chain.

In a related aspect the invention provides a method for promoting neuronal growth or neuronal repair to treat a neurological disorder in a subject, the method comprising administering a polypeptide to the subject, wherein the polypeptide comprises a catalytically inactive clostridial neurotoxin L-chain.

In another related aspect the invention provides use of a polypeptide comprising a catalytically inactive clostridial neurotoxin L-chain in the manufacture of a medicament for promoting neuronal growth or neuronal repair to treat a neurological disorder in a subject.

In one aspect, the invention provides a polypeptide for use in treating a neurological disorder in a subject, wherein the polypeptide comprises a catalytically inactive clostridial neurotoxin L-chain.

In a related aspect the invention provides a method for treating a neurological disorder in a subject, the method comprising administering a polypeptide to the subject, wherein the polypeptide comprises a catalytically inactive clostridial neurotoxin L-chain.

In another related aspect the invention provides use of a polypeptide comprising a catalytically inactive clostridial neurotoxin L-chain in the manufacture of a medicament for treating a neurological disorder in a subject.

The present inventors were the first to show that the catalytic activity of a clostridial neurotoxin L-chain is not necessary to promote neuronal growth or neuronal repair. Thus, the present invention allows for the provision of a safer (less toxic) therapeutic.

Active clostridial neurotoxin L-chain has non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP-25, synaptobrevin/VAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain, for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

Cell-based and in vivo assays may also be used to determine if a clostridial neurotoxin comprising an L-chain and a functional cell binding and translocation domain has non-cytotoxic protease activity. Assays such as the Digit Abduction Score (DAS), the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Donald et al (2018), Pharmacol Res Perspect, e00446, 1-14, which is incorporated herein by reference.

A catalytically inactive L-chain may have one or more mutations that inactivate said catalytic activity. For example, a catalytically inactive BoNT/A L-chain may comprise a mutation of an active site residue, such as His223, Glu224, His227, Glu262, and/or Tyr366. The position numbering corresponds to the amino acid positions of SEQ ID NO: 62 and can be determined by aligning a polypeptide with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. His223 will be His223 of SEQ ID NO: 62). Alternatively, where the methionine is absent from SEQ ID NO: 62 the amino acid residue numbering should be modified by −1 (e.g. His223 will be His222 of SEQ ID NO: 62). Similar considerations apply when the methionine at position 1 of the other polypeptide sequences described herein is present/absent, and the skilled person will readily determine the correct amino acid residue numbering using techniques routine in the art.

In a particularly preferred embodiment, a polypeptide of the invention may comprise a modified BoNT/A or fragment thereof (preferably a BoNT/A H_{C} domain or fragment thereof). The modified BoNT/A or fragment thereof may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such a modified BoNT/A or fragment thereof may demonstrate a reduction in, or absence of, side effects compared to the use of known BoNT/A. The increased tissue retention properties of the modified BoNT/A of the invention may also provide increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 (as well as the SEQ ID NOs corresponding to modified BoNT/A polypeptides or fragments thereof described herein) is optional, the skilled person will take the presence/absence of the methionine residue into account when determining amino acid residue numbering. For example, where SEQ ID NO: 62 includes a methionine, the position numbering will be as defined above (e.g. ASN 886 will be ASN 886 of SEQ ID NO: 62). Alternatively, where the methionine is absent from SEQ ID NO: 2 the amino acid residue numbering should be modified by −1 (e.g. ASN 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

The amino acid residue(s) indicated for modification above are surface exposed amino acid residue(s).

A modified BoNT/A or fragment thereof may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of modified BoNT/A or fragment thereof preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A or fragment thereof may comprise 1-30, 3-20, or 5-10 amino acid modifications. More preferably, the term “one or more amino acid residue(s)” when used in the context of modified BoNT/A or fragment thereof means all of the indicated amino acid residue(s).

Preferably, beyond the one or more amino acid modification(s) at the indicated amino acid residue(s), the modified BoNT/A or fragment thereof does not contain any further amino acid modifications when compared to SEQ ID NO: 62.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A or fragment thereof that has an increased positive surface charge and increased isoelectric point when compared to the corresponding unmodified BoNT/A or fragment thereof.

The isoelectric point (pl) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pl). The isoelectric point is a standard concept in protein biochemistry with which the skilled person would be familiar.

The isoelectric point (pl) is therefore defined as the pH value at which a protein displays a net charge of zero. An increase in pl means that a higher pH value is required for the protein to display a net charge of zero. Thus, an increase in pl represents an increase in the net positive charge of a protein at a given pH. Conversely, a decrease in pl means that a lower pH value is required for the protein to display a net charge of zero. Thus, a decrease in pl represents a decrease in the net positive charge of a protein at a given pH.

Methods of determining the pl of a protein are known in the art and would be familiar to a skilled person. By way of example, the pl of a protein can be calculated from the average pKa values of each amino acid present in the protein (“calculated pl”). Such calculations can be performed using computer programs known in the art, such as the Compute pl/MW Tool from ExPASy (https://web.expasy.org/compute_pi/), which is the preferred method for calculating pl in accordance with the present invention. Comparisons of pl values between different molecules should be made using the same calculation technique/program.

Where appropriate, the calculated pl of a protein can be confirmed experimentally using the technique of isoelectric focusing (“observed pl”). This technique uses electrophoresis to separate proteins according to their pl. Isoelectric focusing is typically performed using a gel that has an immobilised pH gradient. When an electric field is applied, the protein migrates through the pH gradient until it reaches the pH at which it has zero net charge, this point being the pl of the protein. Results provided by isoelectric focusing are typically relatively low-resolution in nature, and thus the present inventors believe that results provided by calculated pl (as described above) are more appropriate to use.

Throughout the present specification, “pl” means “calculated pl” unless otherwise stated.

The pl of a protein may be increased or decreased by altering the number of basic and/or acidic groups displayed on its surface. This can be achieved by modifying one or more amino acids of the protein. For example, an increase in pl may be provided by reducing the number of acidic residues, or by increasing the number of basic residues.

A modified BoNT/A or fragment thereof of the invention may have a pl value that is at least 0.2, 0.4, 0.5 or 1 pl units higher than that of an unmodified BoNT/A (e.g. SEQ ID NO: 62) or fragment thereof. Preferably, a modified BoNT/A or fragment thereof may have a pl of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence or fragment thereof, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A or fragment thereof. In an amino acid substitution, an amino acid residue that forms part of the BoNT/A polypeptide sequence or fragment thereof is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Preferably, a modified BoNT/A or fragment thereof for use in the invention comprises between 4 and 40 amino acid modifications located in the clostridial toxin H_{N} domain. Said modified BoNT/A or fragment thereof preferably also has pl of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A or fragment thereof may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

In one aspect the invention provides a polypeptide for use in promoting neuronal growth or neuronal repair to treat a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In a related aspect, there is provided a method for promoting neuronal growth or neuronal repair to treat a neurological disorder in a subject, the method comprising administering a polypeptide to the subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In a further related aspect, there is provided use of a polypeptide in the manufacture of a medicament for promoting neuronal growth or neuronal repair to treat a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In one aspect the invention provides a polypeptide for use in treating a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In a related aspect, there is provided a method for treating a neurological disorder in a subject, the method comprising administering a polypeptide to the subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In a further related aspect, there is provided use of a polypeptide in the manufacture of a medicament for treating a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.

In one embodiment a polypeptide for use according to the invention comprises a polypeptide sequence having at least 80%, 90%, 95% or 98% sequence identity to SEQ ID NO: 42. Preferably, a polypeptide for use according to the invention comprises a polypeptide sequence shown as SEQ ID NO: 42.

In one embodiment a polypeptide for use according to the invention comprises a polypeptide sequence that is encoded by a nucleotide sequence having at least 80%, 90%, 95% or 98% sequence identity to SEQ ID NO: 41. Preferably, a polypeptide for use according to the invention comprises a polypeptide sequence that is encoded by a nucleotide sequence shown as SEQ ID NO: 41.

In one embodiment a polypeptide for use according to the invention (e.g. comprising SEQ ID NO: 42 or encoded by SEQ ID NO: 41) may be a portion of a polypeptide having at least 70% sequence identity to SEQ ID NO: 61 or 65. Thus, in one embodiment a polypeptide for use according to the invention may comprise a polypeptide sequence having at least 80%, 90%, 95% or 98% sequence identity to SEQ ID NO: 61 or 65. Preferably, a polypeptide for use according to the invention may comprise (more preferably consist of) SEQ ID NO: 61 or 65. In one embodiment the polypeptide comprises a catalytically-inactive L-chain (e.g. as per SEQ ID NO: 65).

In one embodiment a polypeptide for use according to the invention (e.g. comprising SEQ ID NO: 42 or encoded by SEQ ID NO: 41) may be encoded by a nucleotide sequence having at least 70% sequence identity to SEQ ID NO: 60. Thus, in one embodiment a polypeptide for use according to the invention may be encoded by a nucleotide sequence having at least 80%, 90%, 95% or 98% sequence identity to SEQ ID NO: 60. Preferably, a polypeptide for use according to the invention may be encoded by a nucleotide sequence comprising (more preferably consisting of) SEQ ID NO: 60. In one embodiment the polypeptide comprises a catalytically-inactive L-chain.

SEQ ID NO: 42 is an example of a modified BoNT/A fragment and SEQ ID NOs: 61 and 65 are examples of modified BoNT/A polypeptides that are catalytically active and inactive, respectively. Such modified BoNT/A polypeptides and fragments are particularly preferred for use in the present invention. The polypeptides shown as SEQ ID NO: 42, 61 and 62 have a number of amino acid modifications (e.g. substitutions) when compared to wild-type BoNT/A, which increase the isoelectric point of the polypeptide. Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action. Thus, it is envisaged that neuronal growth and/or repair properties of SEQ ID NO: 42, 61 and 65 will be improved compared to equivalent polypeptides lacking said modifications.

For the catalytically active modified BoNT/A polypeptides described above (e.g. SEQ ID NO: 61), one way in which these advantageous properties (which represent an increase in the therapeutic index) may be defined is in terms of the Safety Ratio of the modified BoNT/A. In this regard, undesired effects of a clostridial neurotoxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial neurotoxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki K R, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A catalytically active modified BoNT/A of the present invention may have a Safety Ratio that is higher than the Safety Ratio of an equivalent unmodified (native) botulinum toxin (e.g. SEQ ID NO: 62).

Thus, in one embodiment, a catalytically active modified BoNT/A of the present invention has a Safety Ratio of at least 8 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2].

In one embodiment, a catalytically active modified BoNT/A of the present invention has a Safety Ratio of at least 10. In one embodiment, a modified BoNT/A or fragment thereof of the present invention has a Safety Ratio of at least 15.

Polypeptides comprising at least 70% sequence identity to SEQ ID NO: 61 are described in WO 2015/004461 A1, which is incorporated herein by reference in its entirety.

In one embodiment a polypeptide comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises a substitution at one or more (preferably two or more, three or more, four or more, five or more or six or more, more preferably at all) of positions 930, 955, 991, 1026, 1052, 1229, and 886. The position numbering corresponds to the positions of SEQ ID NO: 62 and can be determined by aligning the polypeptide sequence with SEQ ID NO: 62 (unmodified/wild-type BoNT/A). As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. position 886 will be ASN 886 of SEQ ID NO: 62). Alternatively, where the methionine is absent from SEQ ID NO: 62 the amino acid residue numbering should be modified by −1 (e.g. position 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

Preferably, the polypeptide comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises lysine or arginine (more preferably lysine) at one or more of positions 930, 955, 991, 1026, 1052, 1229, and 886. In one embodiment, the polypeptide comprises lysine or arginine (more preferably lysine) at least two, three, four, five, six or all of positions 930, 955, 991, 1026, 1052, 1229, and 886. Most preferably, the polypeptide comprises lysine or arginine (more preferably lysine) at all of positions 930, 955, 991, 1026, 1052, 1229, and 886.

The polypeptides of the invention promote neuronal growth and/or neuronal repair. Thus, said polypeptides find utility in treating neurological disorders. The term “neurological disorder” as used herein is a disorder that can be treated by promoting neuronal growth and/or repair in a subject.

Thus, in one aspect the invention provides a method for promoting neuronal growth and/or neuronal repair, the method comprising administering a polypeptide to a subject, the polypeptide comprising a clostridial neurotoxin light chain (L-chain) or fragment thereof; and/or a fragment of a clostridial neurotoxin heavy chain (H-chain). In another aspect, the invention provides a method for promoting neuronal growth and/or neuronal repair, the method comprising administering a polypeptide to a subject, the polypeptide comprising a catalytically inactive clostridial neurotoxin L-chain. In another aspect, there is provided a method for promoting neuronal growth or neuronal repair, the method comprising administering a polypeptide to a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41. In another aspect there is provided a method for promoting neuronal growth or neuronal repair, the method comprising administering a polypeptide to a subject, wherein the polypeptide comprises a polypeptide sequence having at least 70% sequence identity to SEQ ID NO: 63.

The term “promotes neuronal growth and/or neuronal repair” may mean that the polypeptide of the invention initiates neuronal growth and/or neuronal repair, for example where neuronal growth and/or neuronal repair was not occurring. In other embodiments, the term “promotes neuronal growth and/or neuronal repair” may mean that the polypeptide of the invention increases the rate of neuronal growth and/or neuronal repair. Said increase may be an increase when compared to the rate of neuronal growth and/or neuronal repair in the absence of the polypeptide of the invention. In one embodiment neuronal growth and/or neuronal repair allows for the rebuilding of damaged neuronal circuits, thereby restoring activity and/or neuronal communication in a network or population of neurons. Thus, the term “neuronal repair” as used herein may encompass repair of a specific neuron as well as repair of a neuronal circuit.

The term “neuronal growth and/or neuronal repair” may also encompass neuronal plasticity. Thus, in one embodiment a polypeptide of the invention promotes neuronal plasticity. The term “neuronal plasticity” as used herein encompasses axonal sprouting, dendritic sprouting, neurogenesis (e.g. the production of new neurons), maturation, differentiation, and/or synaptic plasticity (e.g. including changes to synaptic strength, activity, anatomy, and/or connectivity). In one embodiment a polypeptide of the invention promotes the establishment of functional synapses (e.g. at or near to a site of injury).

Neuronal growth and/or repair may be increased by at least 10%, 20%, 30%, 40%, 50%, 60% or 70% (preferably at least 80%) in the presence of a polypeptide of the invention when compared to the neuronal growth and/or repair in the absence of the polypeptide of the invention or in the presence of an alternative polypeptide. In some embodiments neuronal growth and/or repair may be increased by at least 100%, 150% or 200% in the presence of a polypeptide of the invention when compared to the neuronal growth and/or repair in the absence of the polypeptide of the invention or in the presence of an alternative polypeptide.

In one embodiment, a polypeptide of the invention promotes neuronal growth. The term “neuronal growth” as used herein encompasses growth of any part of a neuron, including growth of axons and/or dendrites. A polypeptide of the invention may increase neurite length, neurite number (e.g. number of neurites per cell), and/or may increase the length and/or numbers of projections from a cell body or cell membrane of a neuron. Preferably, a polypeptide of the invention promotes axonal growth of a neuron, e.g. a neuron in a subject. In other words, preferably a polypeptide of the invention increases axonal growth, e.g. axonal sprouting. Said axonal growth may promote connections and/or chemical communication between neurons.

A neurological disorder treated by a polypeptide of the invention may be a neuronal injury, a neurodegenerative disorder, a sensory disorder or an autonomic disorder.

A neurological disorder may be a neuronal injury. In one embodiment, a neuronal injury may be nerve trauma, neuropathy (e.g. peripheral neuropathy), spinal cord injury, a nerve section, brain injury (e.g. traumatic brain injury), non-traumatic injury (e.g. stroke or spinal cord infarction), or injury to the brachial plexus, e.g. Erb's palsy or Klumpke's palsy.

In one embodiment the nerve trauma may result from scarring and/or from a bone fracture. In such instances of nerve trauma, nerve terminals are damaged. The polypeptide of the invention, advantageously, allows for repair of said nerve terminals or of distal nerve terminals allowing treatment of nerve trauma.

A neuronal injury may be paralysis, such as paralysis caused by spinal cord injury (e.g. caused by compression, constriction, and/or stretching). In one embodiment a spinal cord injury is paraplegia or tetraplegia.

A neurological disorder may be a sensory disorder. In one embodiment, a sensory disorder is sensory neuropathy, sensorimotor polyneuropathy, diabetic neuropathy, pain, Brown-Sequard syndrome, Charcot-Marie-Tooth disease, or Devic's syndrome. Preferably, a sensory disorder described herein is not pain. In other words, preferably a neurological disorder described herein is not pain.

A neurological disorder may be an autonomic disorder. In one embodiment, an autonomic disorder is autonomic neuropathy, multiple system atrophy, acute idiopathic polyneuropathy, dysautonomia, familial dysautonomia, diabetic autonomic failure, pure autonomic failure, temperature regulation disorders, hyperhidrosis, neurally mediated syncope (vasovagal, micturition, cough, swallow and other situational forms), erectile dysfunction, orthostatic hypotension, postural tachycardia syndrome (PoTS), or Guillain-Barre syndrome.

A neurological disorder may be a neurodegenerative disorder. In one embodiment, a neurodegenerative disorder is Alzheimer's disease, Parkinson's disease, Parkinson's disease related disorders, motor neuron disease, peripheral neuropathy, motor neuropathy, prion disease, Huntington's disease, spinocerebellar ataxia, spinal muscular atrophy, monomelic amyotrophy, Friedreich's ataxia, Hallervorden-Spatz disease, or frontotemporal lobar degeneration. Preferably, a neurodegenerative disorder is Parkinson's disease or motor neuron disease. Advantageously, the polypeptides of the invention are believed to find utility in the treatment of neurodegenerative disorders owing to their ability to promote neuronal growth (e.g. including neuronal plasticity) and/or neuronal repair, and further owing to their ability to rebuild damaged neuronal circuits, thereby restoring activity and/or neuronal communication in a network or population of neurons.

The polypeptides of the invention may be considered neurotrophic polypeptides in view of their ability to promote neuronal growth and/or neuronal repair. A neuron described herein may be one or more selected from: a motor neuron (including an autonomic neuron), a sensory neuron, a spinal interneuron, and a cerebral interneuron. Thus, in one embodiment a polypeptide of the invention promotes the growth and/or repair of a motor neuron, a sensory neuron, and/or an interneuron. Preferably, a polypeptide of the invention promotes the growth and/or repair of a motor neuron.

A “subject” as used herein may be a mammal, such as a human or other mammal. Preferably “subject” means a human subject.

The term “disorder” as used herein also encompasses a “disease”. In one embodiment the disorder is a disease.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment.

The term “treat” or “treating” as used herein refers to the disorder and/or a symptom thereof.

Therefore a polypeptide of the invention may be administered to a subject in a therapeutically effective amount or a prophylactically effective amount. Preferably a polypeptide of the invention is administered to a subject in a therapeutically effective amount.

A “therapeutically effective amount” is any amount of the polypeptide, which when administered alone or in combination to a subject for treating said disorder (or a symptom thereof) is sufficient to effect such treatment of the disorder, or symptom thereof.

A “prophylactically effective amount” is any amount of the polypeptide that, when administered alone or in combination to a subject inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely.

The polypeptides of the invention may be formulated in any suitable manner for administration to a subject, for example as part of a pharmaceutical composition. Thus, in one aspect, the invention provides a pharmaceutical composition comprising a polypeptide of the invention and a pharmaceutically acceptable carrier, excipient, adjuvant, propellant and/or salt. In some embodiments, the polypeptide of the invention may be in a single-chain form, while in other embodiments the polypeptide may be in a di-chain form, e.g. where the two chains are linked by a di-sulphide bridge. Preferably the polypeptide is in a di-chain form.

The polypeptides of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a polypeptide that is to be delivered locally, the polypeptide may be formulated as a cream (e.g. for topical application), or for sub-dermal injection.

Local delivery means may include an aerosol, or other spray (e.g. a nebuliser). In this regard, an aerosol formulation of a polypeptide enables delivery to the lungs and/or other nasal and/or bronchial or airway passages.

Polypeptides of the invention may be administered to a subject by intrathecal or epidural injection in the spinal column at the level of the spinal segment involved in the innervation of an affected organ.

A route of administration may be via laproscopic and/or localised injection. In one embodiment a polypeptide of the invention is administered at or near to a site of injury, preferably at a site of injury. For example, where an injury is a spinal cord injury, the polypeptide may be administered intrathecally or intraspinally (preferably intrathecally). In one embodiment the route of administration of a polypeptide of the invention may be perineural, intraneural, intraspinal, and/or intrathecal.

The dosage ranges for administration of the polypeptides of the present invention are those to produce the desired therapeutic and/or prophylactic effect. It will be appreciated that the dosage range required depends on the precise nature of the clostridial neurotoxin or composition, the route of administration, the nature of the formulation, the age of the subject, the nature, extent or severity of the subject's condition, contraindications, if any, and the judgement of the attending physician. Variations in these dosage levels can be adjusted using standard empirical routines for optimisation.

In one embodiment a dosage of the polypeptide is a flat dose. A flat dose may be in the range of 50 pg to 250 ug, preferably 100 pg to 100 ug. In one embodiment a flat dose may be at least 50 pg, 100 pg, 500 pg, 1 ng, 50 ng, 100 ng, 500 ng, 1 ug or 50 ug. Said dose may be a single flat dose.

Fluid dosage forms are typically prepared utilising the polypeptide and a pyrogen-free sterile vehicle. The clostridial neurotoxin, depending on the vehicle and concentration used, can be either dissolved or suspended in the vehicle. In preparing solutions the polypeptide can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

A polypeptide of the invention may be a clostridial neurotoxin or a fragment thereof, preferably a fragment thereof.

In one embodiment, a polypeptide of the invention may be encoded by a nucleotide sequence having at least 70% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. In one embodiment, a polypeptide of the invention may be encoded by a nucleotide sequence having at least 80%, 90%, 95% or 98% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. Preferably, a polypeptide of the invention may be encoded by a nucleotide sequence comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60.

In one embodiment a polypeptide of the invention may comprise a polypeptide sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. In one embodiment a polypeptide of the invention may comprise a polypeptide sequence having at least 80%, 90%, 95% or 98% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. Preferably, a polypeptide of the invention may comprise a polypeptide sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.

In one embodiment the present invention encompasses the use of full-length clostridial neurotoxins comprising a clostridial neurotoxin L-chain and a clostridial neurotoxin H-chain with the proviso that said clostridial neurotoxin L-chain is catalytically inactive.

The term “clostridial neurotoxin” embraces toxins produced by C. botulinum (botulinum neurotoxin serotypes A, B, C1, D, E, F, G, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing.

Botulinum neurotoxin (BoNT) is produced by C. botulinum in the form of a large protein complex, consisting of BoNT itself complexed to a number of accessory proteins. There are at present eight different classes of botulinum neurotoxin, namely: botulinum neurotoxin serotypes A, B, C1, D, E, F, G, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the clostridial neurotoxin of the invention is a modified clostridial neurotoxin, or a modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL, xExxxLL, xExxxlL, and xExxxLM (wherein x is any amino acid). Suitable tyrosine-based motifs include Y-x-x-Hy (wherein Hy is a hydrophobic amino acid). Examples of modified clostridial neurotoxins comprising leucine- and tyrosine-based motifs are described in WO 2002/08268, which is hereby incorporated by reference in its entirety.

As described above, a modified clostridial neurotoxin (or clostridial neurotoxin fragment) may be one that comprises one or more modifications that increases the isoelectric point of the clostridial neurotoxin when compared to an equivalent unmodified clostridial neurotoxin lacking said one or more modifications. Suitable modified clostridial neurotoxins are described above and in WO 2015/004461 A1 and WO 2016/110662 A1, which are incorporated herein by reference. Exemplary sequences include SEQ ID NOs: 61 and 42 described herein.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the clostridial neurotoxin (or fragment thereof) of the invention is a hybrid clostridial neurotoxin, or a chimeric clostridial neurotoxin.

In a particularly preferred embodiment, a polypeptide of the invention may be a chimeric clostridial neurotoxin comprising (preferably consisting of) a BoNT/A light-chain and translocation domain, and a BoNT/B receptor binding domain (H_{C} domain) or a portion thereof. A suitable chimeric and/or hybrid clostridial neurotoxin may be one taught in WO 2017/191315 A1, which is incorporated herein by reference. Such preferred sequences include SEQ ID NOs: 44, 63, and 64.

The BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C=O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a 6-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureld=3BTA) and 1 EPW (http://www.rcsb.org/pdb/explore/explore.do?structureld=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{C}N” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{C}N domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 31c, helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀, helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 62) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (a.k.a. SEQ | | | | |
| ID NO: 52) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8th residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

The LH_{N} domain from BoNT/A may correspond to amino acid residues 1 to 872 of SEQ ID NO: 62, or a polypeptide sequence having at least 70% sequence identity thereto. The LH_{N} domain from BoNT/A may correspond to amino acid residues 1 to 872 of SEQ ID NO: 62, or a polypeptide sequence having at least 80%, 90% or 95% sequence identity thereto. Preferably, the LH_{N} domain from BoNT/A corresponds to amino acid residues 1 to 872 of SEQ ID NO: 62.

The H_{C} domain from BoNT/B may correspond to amino acid residues 860 to 1291 of SEQ ID NO: 52, or a polypeptide sequence having at least 70% sequence identity thereto. The H_{C} domain from BoNT/B may correspond to amino acid residues 860 to 1291 of SEQ ID NO: 52, or a polypeptide sequence having at least 80%, 90% or 95% sequence identity thereto. Preferably, the H_{C} domain from BoNT/B corresponds to amino acid residues 860 to 1291 of SEQ ID NO: 52.

Preferably, the BoNT/AB chimera comprises a BoNT/A LH_{N} domain and a BoNT/B H_{C} domain. More preferably, the LH_{N} domain corresponds to amino acid residues 1 to 872 of BoNT/A (SEQ ID NO: 62) and the H_{C} domain corresponds to amino acid residues 860 to 1291 of BoNT/B (SEQ ID NO: 52).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E11911; E1191Q; E1191T; 51199Y; 51199F; 51199L; S1201V; E1191C, E1191V, E1191L, E1191Y, S1199W, 51199E, 51199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and 51199L, E1191M and 51199Y, E1191M and 51199F, E1191Q and 51199L, E1191Q and 51199Y, E1191Q and 51199F, E1191M and S1199W, E1191M and W1178Q, E1191C and S1199W, E1191C and 51199Y, E1191C and W1178Q, E1191Q and S1199W, E1191V and S1199W, E1191V and 51199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and 51199Y.

The modification may be a modification when compared to unmodified BoNT/B shown as SEQ ID NO: 52, wherein the amino acid residue numbering is determined by alignment with SEQ ID NO: 52. As the presence of a methionine residue at position 1 of SEQ ID NO: 52 is optional, the skilled person will take the presence/absence of the methionine residue into account when determining amino acid residue numbering. For example, where SEQ ID NO: 52 includes a methionine, the position numbering will be as defined above (e.g. E1191 will be E1191 of SEQ ID NO: 52). Alternatively, where the methionine is absent from SEQ ID NO: 52 the amino acid residue numbering should be modified by −1 (e.g. E1191 will be E1190 of SEQ ID NO: 52). Similar considerations apply when the methionine at position 1 of the other polypeptide sequences described herein is present/absent, and the skilled person will readily determine the correct amino acid residue numbering using techniques routine in the art.

Thus, in one aspect, the invention provides a polypeptide for use in promoting neuronal growth or neuronal repair to treat a neurological disorder in a subject, wherein the polypeptide comprises a polypeptide sequence having at least 70% sequence identity to SEQ ID NO: 63 or 64.

In a related aspect, there is provided a method for promoting neuronal growth or neuronal repair to treat a neurological disorder in a subject, the method comprising administering a polypeptide to the subject, wherein the polypeptide comprises a polypeptide sequence having at least 70% sequence identity to SEQ ID NO: 63 or 64.

In a further related aspect, there is provided use of a polypeptide in the manufacture of a medicament for promoting neuronal growth or neuronal repair to treat a neurological disorder in a subject, wherein the polypeptide comprises a polypeptide sequence having at least 70% sequence identity to SEQ ID NO: 63 or 64.

In one aspect the invention provides a polypeptide for use in treating a neurological disorder in a subject, wherein the polypeptide comprises a polypeptide sequence having at least 70% sequence identity to SEQ ID NO: 63 or 64.

In a related aspect, there is provided a method for treating a neurological disorder in a subject, the method comprising administering a polypeptide to the subject, wherein the polypeptide comprises a polypeptide sequence having at least 70% sequence identity to SEQ ID NO: 63 or 64.

In a further related aspect, there is provided use of a polypeptide in the manufacture of a medicament for treating a neurological disorder in a subject, wherein the polypeptide comprises a polypeptide sequence having at least 70% sequence identity to SEQ ID NO: 63 or 64.

In one embodiment a polypeptide for use according to the invention comprises a polypeptide sequence having at least 80%, 90%, 95% or 98% sequence identity to SEQ ID NO: 63 or 64. Preferably, a polypeptide for use according to the invention comprises (more preferably consists of) a polypeptide sequence shown as SEQ ID NO: 63 or 64.

Preferably, the polypeptide comprising a polypeptide sequence having at least 70% sequence identity to SEQ ID NO: 63 comprises a catalytically-inactive L-chain, such as SEQ ID NO: 64.

A chimeric and/or hybrid clostridial neurotoxin for use in the present invention may comprise a portion of a BoNT/A polypeptide and a portion of a BoNT/B polypeptide, an example of which includes the polypeptide described herein as SEQ ID NO: 44.

Suitable chimeric clostridial neurotoxins may include BoNT/FA. Indeed, in a particularly preferred embodiment, a polypeptide of the invention may comprise BoNT/FA or a fragment thereof. Catalytically inactive forms of BoNT/FA are described herein as SEQ ID NO: 26 and 34. Suitable fragments of BoNT/FA are also described herein as SEQ ID NOs: 28, 30, and 32.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: 0T022244.1), which cleaves VAMP2 and SNAP25, and the Chryseobacterium pipero encoded toxin (NCBI Ref.Seq: WP_034687872.1).

The polypeptide of the present invention may lack a functional H_{C} domain of a clostridial neurotoxin and also lack any functionally equivalent exogenous ligand Targeting Moiety (TM).

Thus, in a particularly preferred embodiment, a clostridial neurotoxin of the invention is not a re-targeted clostridial neurotoxin. In a re-targeted clostridial neurotoxin, the clostridial neurotoxin is modified to include an exogenous ligand known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety.

As discussed above, (full-length) clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

A clostridial neurotoxin may be selected from BoNT/A, BoNT/B, BoNT/C, BoNT/D, BoNT/E, BoNT/F, BoNT/G, BoNT/X, and TeNT (tetanus neurotoxin). Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X.

In one embodiment the clostridial neurotoxin may be BoNT/A. A reference BoNT/A sequence is shown as SEQ ID NO: 51. In another embodiment the clostridial neurotoxin may be BoNT/B. A reference BoNT/B sequence is shown as SEQ ID NO: 52. In another embodiment the clostridial neurotoxin may be BoNT/C. A reference BoNT/C sequence is shown as SEQ ID NO: 53. In another embodiment the clostridial neurotoxin may be BoNT/D. A reference BoNT/D sequence is shown as SEQ ID NO: 54. In another embodiment the clostridial neurotoxin may be BoNT/E. A reference BoNT/E sequence is shown as SEQ ID NO: 55. In another embodiment the clostridial neurotoxin may be BoNT/F. A reference BoNT/F sequence is shown as SEQ ID NO: 56. In another embodiment the clostridial neurotoxin may be BoNT/G. A reference BoNT/G sequence is shown as SEQ ID NO: 57. In another embodiment the clostridial neurotoxin may be TeNT. A reference TeNT sequence is shown as SEQ ID NO: 58. In another embodiment the clostridial neurotoxin may be BoNT/X. A reference BoNT/X sequence is shown as SEQ ID NO: 59.

In one embodiment a polypeptide of the invention comprises a fragment of a BoNT/A or a fragment of a BoNT/F. In another embodiment, the polypeptide of the invention comprises a catalytically inactive L-chain of BoNT/A or BoNT/F.

In embodiments where a polypeptide described herein has a tag for purification (e.g. a His-tag) and/or a linker, said tag and/or linker are optional.

Suitable full-length clostridial neurotoxins are described herein.

In one embodiment a polypeptide of the invention may comprise a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65 with the proviso that a clostridial neurotoxin L-chain of said polypeptide is catalytically inactive. In one embodiment a polypeptide of the invention may comprise a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65 with the proviso that a clostridial neurotoxin L-chain of said polypeptide is catalytically inactive. Preferably, a polypeptide of the invention may comprise a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65 with the proviso that a clostridial neurotoxin L-chain of said polypeptide is catalytically inactive.

In one embodiment a polypeptide of the invention may be one encoded by a nucleotide sequence having at least 70% sequence identity to any one of SEQ ID NOs: 1, 9, 11, 13, 15, 17, 25, 33, or 60 with the proviso that the clostridial neurotoxin L-chain of said polypeptide is catalytically inactive. In one embodiment a polypeptide of the invention is one encoded by a nucleotide sequence having at least 80%, 90%, 95% or 98% sequence identity to any one of SEQ ID NOs: 1, 9, 11, 13, 15, 17, 25, 33, or 60 with the proviso that the clostridial neurotoxin L-chain of said polypeptide is catalytically inactive. Preferably, a polypeptide of the invention is one encoded by a nucleotide sequence comprising any one of SEQ ID NOs: 1, 9, 11, 13, 15, 17, 25, 33, or 60 with the proviso that the clostridial neurotoxin L-chain of said polypeptide is catalytically inactive.

In one embodiment a polypeptide of the invention may comprise a polypeptide sequence having at least 70% sequence identity to any one of SEQ ID NOs: 2, 10, 12, 14, 16, 18, 26, 34, 64 or 65 with the proviso that the clostridial neurotoxin L-chain of said polypeptide is catalytically inactive. In one embodiment a polypeptide of the invention comprises a polypeptide sequence having at least 80%, 90%, 95% or 98% sequence identity to any one of SEQ ID NOs: 2, 10, 12, 14, 16, 18, 26, 34, 64 or 65 with the proviso that the clostridial neurotoxin L-chain of said polypeptide is catalytically inactive. Preferably, a polypeptide of the invention comprises any one of SEQ ID NOs: 2, 10, 12, 14, 16, 18, 26, 34, 64 or 65 with the proviso that the clostridial neurotoxin L-chain of said polypeptide is catalytically inactive.

In one embodiment a polypeptide of the invention is a full-length clostridial neurotoxin selected from BoNT/B, BoNT/C, BoNT/D, BoNT/E, BoNT/F, BoNT/G, BoNT/X, and TeNT.

In one embodiment a polypeptide of the invention may comprise a polypeptide sequence having at least 70% sequence identity to any one of SEQ ID NOs: 52-59, 61 or 63. In one embodiment a polypeptide of the invention may comprise a polypeptide sequence having at least 80%, 90%, 95% or 98% sequence identity to any one of SEQ ID NOs: 52-59, 61 or 63. In one embodiment a polypeptide of the invention may comprise a polypeptide sequence having at least 99% or 99.9% sequence identity to any one of SEQ ID NOs: 52-59, 61 or 63. Preferably, a polypeptide of the invention may comprise (more preferably consist of) a polypeptide sequence comprising any one of SEQ ID NOs: 52-59, 61 or 63.

In a particularly preferred embodiment a polypeptide of the invention is not a full-length catalytically active clostridial neurotoxin, e.g. is not full-length catalytically active BoNT/A.

The polypeptide of the present invention may comprise (or consist of) a fragment of a clostridial neurotoxin, e.g. a fragment of any full-length clostridial neurotoxin described herein.

In one embodiment a polypeptide of the invention may comprise a fragment of a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65. In one embodiment a polypeptide of the invention may comprise a fragment of a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65. Preferably, a polypeptide of the invention may comprise a fragment of a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65.

In one embodiment a polypeptide of the invention comprises (or consists of) a clostridial neurotoxin L-chain or fragment thereof. A fragment of a clostridial neurotoxin L-chain may have 400, 350, 300, 250, 200, 150, 100 or 50 amino acid residues of a clostridial neurotoxin L-chain. In one embodiment, a fragment of a clostridial neurotoxin L-chain has at least 20, 30, 40, 50, 60, 70, 80, 90, 100, 120, 150 or 200 amino acid residues of a clostridial neurotoxin L-chain. For example, a fragment of a clostridial neurotoxin L-chain may have 20-400, 50-300 or 100-200 amino acid residues of a clostridial neurotoxin L-chain.

Examples of L-chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Suitable clostridial neurotoxin L-chains are described herein.

A clostridial neurotoxin L-chain may comprise a polypeptide sequence having at least 70% sequence identity to any one of SEQ ID NOs: 6, 24, 32 or 40 or a fragment thereof. In one embodiment a clostridial neurotoxin L-chain comprises a polypeptide sequence having at least 80%, 90%, 95% or 98% sequence identity to any one of SEQ ID NOs: 6, 24, 32 or 40 or a fragment thereof. Preferably, a clostridial neurotoxin L-chain comprises (more preferably consists of) a polypeptide sequence comprising any one of SEQ ID NOs: 6, 24, 32 or 40 or a fragment thereof.

A clostridial neurotoxin L-chain may be one encoded by a nucleotide sequence having at least 70% sequence identity to any one of SEQ ID NOs: 5, 23, 31 or 39 or a fragment thereof. In one embodiment a clostridial neurotoxin L-chain is one encoded by a nucleotide sequence having at least 80%, 90%, 95% or 98% sequence identity to any one of SEQ ID NOs: 5, 23, 31 or 39 or a fragment thereof. Preferably, a clostridial neurotoxin L-chain is one encoded by a nucleotide sequence comprising any one of SEQ ID NOs: 5, 23, 31 or 39 or a fragment thereof.

In one embodiment a polypeptide of the invention comprises (or consists of) a fragment of a clostridial neurotoxin H-chain. A fragment of a clostridial neurotoxin H-chain may have 800, 700, 600, 500, 400, 350, 300, 250, 200, 150, 100 or 50 amino acid residues of a clostridial neurotoxin H-chain. In one embodiment, a fragment of a clostridial neurotoxin H-chain has at least 20, 30, 40, 50, 60, 70, 80, 90, 100, 120, 150 or 200 amino acid residues of a clostridial neurotoxin H-chain. For example, a fragment of a clostridial neurotoxin H-chain may have 20-800, 30-600, 40-400, 50-300 or 100-200 amino acid residues of a clostridial neurotoxin H-chain.

A clostridial neurotoxin H-chain comprises two structural/functional domains: the translocation domain (H_{N}) and receptor binding domain (H_{C}).

In one embodiment a polypeptide of the invention comprises (or consists of) a clostridial neurotoxin translocation domain or a fragment thereof. A fragment of a clostridial neurotoxin translocation domain may have 400, 350, 300, 250, 200, 150, 100 or 50 amino acid residues of a clostridial neurotoxin translocation domain. In one embodiment, a fragment of a clostridial neurotoxin translocation domain has at least 20, 30, 40, 50, 60, 70, 80, 90, 100, 120, 150 or 200 amino acid residues of a clostridial neurotoxin translocation domain. For example, a fragment of a clostridial neurotoxin translocation domain may have 20-400, 50-300 or 100-200 amino acid residues of a clostridial neurotoxin translocation domain.

The translocation domain is a fragment of the H-chain of a clostridial neurotoxin approximately equivalent to the amino-terminal half of the H-chain, or the domain corresponding to that fragment in the intact H-chain. In one embodiment the H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, in some embodiments the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-5858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (5458-V879)

In the context of the present invention, a variety of clostridial neurotoxin H_{N} regions comprising a translocation domain can be useful in aspects of the present invention. in one embodiment these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising a translocation domain having a length of, for example, at most 350 amino acids, at most 375 amino acids, at most 400 amino acids and at most 425 amino acids.

For further details on the genetic basis of toxin production in Clostridium botulinum and C. tetani, see Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues. In one embodiment said modified H_{N} portions still demonstrate the above-mentioned translocation function.

In a preferred embodiment a polypeptide of the invention comprises (or consists of) a clostridial neurotoxin receptor binding domain (H_{C}) or a fragment thereof. A fragment of a clostridial neurotoxin receptor binding domain (H_{C}) may have 350, 300, 250, 200, 150, 00 or 50 amino acid residues of a clostridial neurotoxin receptor binding domain (H_{C}). In one embodiment, a fragment of a clostridial neurotoxin receptor binding domain (H_{C}) has at least 20, 30, 40, 50, 60, 70, 80, 90, 100, 120, 150 or 200 amino acid residues of a clostridial neurotoxin receptor binding domain (H_{C}). For example, a fragment of a clostridial neurotoxin receptor binding domain (H_{C}) may have 20-350, 50-300 or 100-200 amino acid residues of a clostridial neurotoxin receptor binding domain (H_{C}).

Examples of clostridial neurotoxin receptor binding domain (H_{C}) reference sequences include:
BoNT/A—N872—L1296
BoNT/B—E859—E1291
BoNT/C1—N867-E1291
BoNT/D—S863—E1276
BoNT/E—R846—K1252
BoNT/F—K865—E1274
BoNT/G—N864—E1297
TeNT—1880—D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

A clostridial neurotoxin H-chain may further comprise a translocation facilitating domain. Said domain facilitates delivery of the L-chain into the cytosol of the target cell and are described, for example, in WO 08/008803 and WO 08/008805, each of which is herein incorporated by reference thereto.

By way of example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{C}N translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) clostridial neurotoxin H_{C}N domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Suitable clostridial neurotoxin H_{C} domains are described herein.

A clostridial neurotoxin H_{C} domain may comprise a polypeptide sequence having at least 70% sequence identity to any one of SEQ ID NOs: 8, 22, 30, 38, 42, 44, 46, 48 or 50 or a fragment thereof. In one embodiment a clostridial neurotoxin H_{C} domain comprises a polypeptide sequence having at least 80%, 90%, 95% or 98% sequence identity to any one of SEQ ID NOs: 8, 22, 30, 38, 42, 44, 46, 48 or 50 or a fragment thereof. Preferably, a clostridial neurotoxin H_{C} domain comprises (more preferably consists of) a polypeptide sequence comprising any one of SEQ ID NOs: 8, 22, 30, 38, 42, 44, 46, 48 or 50 or a fragment thereof.

A clostridial neurotoxin H_{C} domain may be one encoded by a nucleotide sequence having at least 70% sequence identity to any one of SEQ ID NOs: 7, 21, 29, 37, 41, 43, 45, 47 or 49 or a fragment thereof. In one embodiment a clostridial neurotoxin H_{C} domain is one encoded by a nucleotide sequence having at least 80%, 90%, 95% or 98% sequence identity to any one of SEQ ID NOs: 7, 21, 29, 37, 41, 43, 45, 47 or 49 or a fragment thereof. Preferably, a clostridial neurotoxin H_{C} domain is one encoded by a nucleotide sequence comprising any one of SEQ ID NOs: 7, 21, 29, 37, 41, 43, 45, 47 or 49 or a fragment thereof.

In one embodiment a clostridial neurotoxin H_{C} domain for use in the invention is a variant BoNT/A H_{C} domain. Said variant BoNT/A H_{C} domain may comprise a modification of one or more amino acids residues selected from Y1117, F1252, H1253, and L1278. For example, a variant BoNT/A H_{C} domain may comprise one or more (preferably two or more) of the following modifications Y1117V, F1252Y, H1253K, and L1278F or L1278H.

In one embodiment a variant BoNT/A H_{C} domain comprises the following modifications: Y1117V and H1253K; or Y1117V, F1252Y, H1253K, and L1278F; or Y1117V, F1252Y, H1253K, and L1278H.

Preferably, a variant BoNT/A H_{C} domain comprises the following modifications: Y1117V and H1253K; or Y1117V, F1252Y, H1253K, and L1278H.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. Y1117 will align against Y1117 of SEQ ID NO: 62). Alternatively, where the methionine is absent from SEQ ID NO: 62 the amino acid residue numbering should be modified by −1 (e.g. Y1117 will align against Y1116 of SEQ ID NO: 52). Similar considerations apply when the methionine at position 1 of the other polypeptide sequences described herein is present/absent, and the skilled person will readily determine the correct amino acid residue numbering using techniques routine in the art.

A variant BoNT/A H_{C} domain may comprise a polypeptide sequence having at least 70% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46, 48 or 50 or a fragment thereof with the proviso that the variant BoNT/A H_{C} domain comprises a modification as described above. Preferably, a variant BoNT/A H_{C} domain comprises (more preferably consists of) a polypeptide sequence comprising any one of SEQ ID NOs: 46, 48 or 50 or a fragment thereof.

A variant BoNT/A H_{C} domain may comprise a polypeptide sequence having at least 70% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46 or 50 or a fragment thereof with the proviso that the variant BoNT/A H_{C} domain comprises a modification as described above. Preferably, a variant BoNT/A H_{C} domain comprises (more preferably consists of) a polypeptide sequence comprising any one of SEQ ID NOs: 46 or 50 or a fragment thereof.

A variant BoNT/A H_{C} domain may be one encoded by a nucleotide sequence having at least 70% sequence identity to any one of SEQ ID NOs: 45, 47 or 49 or a fragment thereof with the proviso that the variant BoNT/A H_{C} domain comprises a modification as described above. In one embodiment a variant BoNT/A H_{C} domain be one encoded by a nucleotide sequence having at least 80%, 90%, 95% or 98% sequence identity to any one of SEQ ID NOs: 45, 47 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45, 47 or 49 or a fragment thereof with the proviso that the variant BoNT/A H_{C} domain comprises a modification as described above. Preferably, a variant BoNT/A H_{C} domain be one encoded by any one of SEQ ID NOs: 45, 47 or 49 or a fragment thereof.

A variant BoNT/A H_{C} domain may be one encoded by a nucleotide sequence having at least 70% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 80%, 90%, 95% or 98% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45 or 49 or a fragment thereof with the proviso that the variant BoNT/A H_{C} domain comprises a modification as described above. Preferably, a variant BoNT/A H_{C} domain be one encoded by any one of SEQ ID NOs: 45 or 49 or a fragment thereof.

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{C}N translocation facilitating domain may be combined with a non-clostridial translocation domain peptide. Alternatively, a clostridial neurotoxin H_{C}N facilitating domain may be combined with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the clostridial neurotoxins of the present invention may lack a functional H_{C} domain of a clostridial neurotoxin. In one embodiment, the clostridial neurotoxins preferably lack the last 50 C-terminal amino acids of a clostridial neurotoxin holotoxin. In another embodiment, the clostridial neurotoxins preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{C}N peptide or domain) and the C-terminal region (commonly referred to as the H_{CC} peptide or domain). This fact is confirmed by the following publications, each of which is herein incorporated in its entirety by reference thereto: Umland TC (1997) Nat. Struct. Biol. 4: 788-792; Herreros J (2000) Biochem. J. 347: 199-204; Halpern J (1993) J. Biol. Chem. 268:15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{cc}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to reduce its native binding ability for nerve terminals at the neuromuscular junction.

Thus, in one embodiment, a clostridial neurotoxin H_{N} peptide of the present invention lacks part of a C-terminal peptide portion (H_{cc}) of a clostridial neurotoxin and thus lacks the H_{C} binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the H_{C} binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

In a preferred embodiment a polypeptide of the invention comprises (or consists of) a clostridial neurotoxin L-chain or fragment thereof and a fragment of a clostridial neurotoxin H-chain. For example, a polypeptide may comprise (or consist of) a clostridial neurotoxin L-chain or fragment thereof and a clostridial neurotoxin translocation domain (H_{N}). Preferably, the polypeptide does not further comprise a clostridial neurotoxin receptor binding domain (H_{C}) or at least the C-terminal portion of a clostridial neurotoxin receptor binding domain (H_{cc}). Thus, in one embodiment a polypeptide of the present invention lacks a C-terminal portion of a clostridial neurotoxin receptor binding domain (H_{cc}). Advantageously, such polypeptides lack the endogenous clostridial neurotoxin receptor binding capabilities and thus exhibit fewer off-target effects in a subject administered said polypeptide.

In one embodiment a polypeptide of the invention consists essentially of a clostridial neurotoxin L-chain or fragment thereof and/or a fragment of a clostridial neurotoxin H-chain. The term “consists essentially of” as used in this context means that the polypeptide does not further comprise one or more amino acid residues that confer additional functionality to the polypeptide, e.g. when administered to a subject. In other words, a polypeptide that “consists essentially of” a clostridial neurotoxin L-chain or fragment thereof and/or a fragment of a clostridial neurotoxin H-chain may further comprise one or more amino acid residues (to those of the clostridial neurotoxin L-chain or fragment thereof and/or fragment of a clostridial neurotoxin H-chain) but said one or more further amino acid residues do not confer additional functionality to the polypeptide, e.g. when administered to a subject. Additional functionality may include enzymatic activity, binding activity and/or any physiological activity whatsoever.

In one embodiment a polypeptide may comprise non-clostridial neurotoxin sequences in addition to any clostridial neurotoxin sequences. The non-clostridial neurotoxin sequences preferably do not disrupt the ability of a polypeptide of the invention to promote neuronal growth or neuronal repair. Preferably, the non-clostridial neurotoxin sequence is not one having catalytic activity, e.g. enzymatic activity. Preferably, the non-clostridial sequence is not one that binds to a cellular receptor. In other words, it is most preferred that the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. It is preferred that any clostridial neurotoxin sequences comprised in said polypeptide consist of a clostridial neurotoxin L-chain or fragment thereof and/or a fragment of a clostridial neurotoxin H-chain. In one embodiment, the clostridial neurotoxin sequence comprised in said polypeptide may consist of a clostridial neurotoxin L-chain. In one embodiment, the clostridial neurotoxin sequence comprised in said polypeptide may consist of a clostridial neurotoxin translocation domain. In one embodiment, the clostridial neurotoxin sequence comprised in said polypeptide may consist of a clostridial neurotoxin receptor binding domain. In one embodiment, the clostridial neurotoxin sequence comprised in said polypeptide may consist of a clostridial neurotoxin L-chain and a clostridial neurotoxin translocation domain.

Suitable polypeptides comprising (or consisting of) a clostridial neurotoxin L-chain and translocation domain are described herein.

A clostridial neurotoxin comprising (or consisting of) a clostridial neurotoxin L-chain and translocation domain may comprise a polypeptide sequence having at least 70% sequence identity to any one of SEQ ID NOs: 4, 20, 28 or 36 or a fragment thereof. In one a clostridial neurotoxin comprising (or consisting of) a clostridial neurotoxin L-chain and translocation domain comprises a polypeptide sequence having at least 80%, 90%, 95% or 98% sequence identity to any one of SEQ ID NOs: 4, 20, 28 or 36 or a fragment thereof. Preferably, a clostridial neurotoxin comprising (or consisting of) a clostridial neurotoxin L-chain and translocation domain comprises (more preferably consists of) a polypeptide sequence comprising any one of SEQ ID NOs: 4, 20, 28 or 36 or a fragment thereof.

A clostridial neurotoxin comprising (or consisting of) a clostridial neurotoxin L-chain and translocation domain may be one encoded by a nucleotide sequence having at least 70% sequence identity to any one of SEQ ID NOs: 3, 19, 27 or 35 or a fragment thereof. In one embodiment a clostridial neurotoxin comprising (or consisting of) a clostridial neurotoxin L-chain and translocation domain is one encoded by a nucleotide sequence having at least 80%, 90%, 95% or 98% sequence identity to any one of SEQ ID NOs: 3, 19, 27 or 35 or a fragment thereof. Preferably, a clostridial neurotoxin comprising (or consisting of) a clostridial neurotoxin L-chain and translocation domain is one encoded by a nucleotide sequence comprising any one of SEQ ID NOs: 3, 19, 27 or 35 or a fragment thereof.

The polypeptides of the present invention may be free from the complexing proteins that are present in a naturally occurring clostridial neurotoxin complex.

The polypeptides of the present invention can be produced using recombinant nucleic acid technologies. Thus, in one embodiment, a polypeptide (as described above) is a recombinant polypeptide.

In one embodiment a nucleic acid (for example, a DNA) comprising a nucleic acid sequence encoding a polypeptide is provided. In one embodiment, the nucleic acid sequence is prepared as part of a DNA vector comprising a promoter and a terminator.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A polypeptide of the invention (and especially any clostridial neurotoxin portion thereof) may be present as a single-chain or as a di-chain.

The invention provides a method of producing a single-chain polypeptide having a light chain and a heavy chain, the method comprising expressing a nucleic acid described herein in an expression host, lysing the host cell to provide a host cell homogenate containing the single-chain polypeptide, and isolating the single-chain polypeptide. In one aspect, the present invention provides a method of activating a polypeptide described herein, the method comprising contacting the polypeptide with a protease that hydrolyses a peptide bond in the activation loop of the polypeptide, thereby converting the (single-chain) polypeptide into a corresponding di-chain polypeptide (e.g. wherein the light chain and heavy chain are joined together by a disulphide bond).

The present invention therefore provides a di-chain polypeptide obtainable by a method of the invention.

Embodiments related to the various therapeutic uses of the invention are intended to be applied equally to methods of treatment, polypeptides of the invention, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}length of the longer sequence plus the \\ number of gaps introduced into the longer \\ sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine

Acidic: glutamic acid
aspartic acid

Polar: glutamine
asparagine

Hydrophobic: leucine
isoleucine
valine

Aromatic: phenylalanine
tryptophan
tyrosine

Small: glycine
alanine
serine
threonine
methionine

In addition to the 20 standard amino acids, non-standard amino acids (such as 4-hydroxyproline, 6-N-methyl lysine, 2-am inoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasm ids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the neurotrophic effect of different recombinantly expressed catalytically inactive BoNT serotypes compared to positive control brain-derived neurotrophic factor (BDNF) in motor-neuron like cell line NSC34. * p<0.05 vs untreated control, one-way ANOVA followed by Dunnett's multiple comparison test. Data are mean±standard error of three independent experiments, each performed in six replicate wells.

FIG. 2 shows the neurotrophic effect of botulinum neurotoxin serotype A fragments in motor-neuron like cell line NSC34 and the effect of recombinantly expressed catalytically inactive BoNT/A. BDNF was used as a positive control. * p<0.05 vs untreated control, one-way ANOVA followed by Dunnett's multiple comparison test. Data are mean±standard error of three independent experiments, each performed in six replicate wells.

FIG. 3 shows the neurotrophic effect of negative controls versus recombinantly expressed catalytically inactive BoNT/A (BoNT/A (0)) in motor-neuron like cell line NSC34. BDNF was used as a positive control. * p<0.05 vs untreated control, one-way ANOVA followed by Dunnett's multiple comparison test. Data are mean±standard error of three independent experiments, each performed in six replicate wells.

FIG. 4 shows the results of a horizontal ladder test for mice administered vehicle control (PBS) or rBoNT/A(0) at 100 pg, 100 ng or 50 ug.

FIG. 5 shows: (A) immunohistochemistry using antibodies binding to neurofilament 200 (NF200) at 4 weeks following administration of vehicle (PBS) (left panel) or 100 ng rBoNT/A(0) (right panel); and (B) immunohistochemistry using antibodies binding to MAP1b at 4 weeks following administration of vehicle (PBS) (left panel) or 100 ng rBoNT/A(0) (right panel). Lesion sites are indicated by * (and for FIG. 5B indicated by white arrows).

FIG. 6 shows the effect of (A) catalytically inactive BoNT/A(0), (B) a BoNT/A light-chain plus translocation domain fragment (LH_{N}/A), (C) BoNT/A light-chain (LC/A, i.e. L/A), and (D) a BoNT/A receptor binding domain (H_{C}/A) on the number of neurites per cell. The BoNT or BoNT fragment was compared to BSA (negative control), BDNF (positive control), and tested at concentrations of 0.1 nM, 1 nM, and 10 nM. * p<0.05 vs BSA control, one-way ANOVA followed by Dunnett's post hoc test. Data are mean±s.e.mean.

FIG. 7 shows the effect of (A) catalytically inactive BoNT/FA(0), (B) a BoNT/FA light-chain plus translocation domain fragment (LH_{N}/FA), (C) BoNT/FA light-chain (LC/FA, i.e. UFA), and (D) a BoNT/FA receptor binding domain (H_{C}/FA) on the number of neurites per cell. The BoNT or BoNT fragment was compared to BSA (negative control), BDNF (positive control), and tested at concentrations of 0.1 nM, 1 nM, and 10 nM. * p<0.05 vs BSA control, one-way ANOVA followed by Dunnett's post hoc test. Data are mean±s.e.mean.

FIG. 8 shows the effect of (A) a BoNT/F light-chain plus translocation domain fragment (LH_{N}/F), (B) BoNT/F light-chain (LC/F, i.e. L/F), and (C) a BoNT/F receptor binding domain (H_{C}/F) on the number of neurites per cell. The BoNT or BoNT fragment was compared to BSA (negative control), BDNF (positive control), and tested at concentrations of 0.1 nM, 1 nM, and 10 nM. * p<0.05 vs BSA control, one-way ANOVA followed by Dunnett's post hoc test. Data are mean±s.e.mean.

FIG. 9 shows the effect of cationic rH_{C}/A (i.e. mrHC/A) on the number of neurites per cell. The cationic BoNT fragment was compared to BSA (negative control), BDNF (positive control), and tested at concentrations of 0.1 nM, 1 nM, and 10 nM. * p<0.05 vs BSA control, one-way ANOVA followed by Dunnett's post hoc test. Data are mean±s.e.mean.

FIG. 10 shows the effect of (A) toxHC/A YH (i.e. rH_{C}/A Variant Y1117V H1253K) and (B) toxHC/A YFHL (L to H) (i.e., rH_{C}/A Variant Y1117V F1252Y H1253K L1278H) on the number of neurites per cell. The variant BoNT fragments were compared to BSA (negative control), BDNF (positive control), and tested at concentrations of 0.1 nM, 1 nM, and 10 nM. * p<0.05 vs BSA control, one-way ANOVA followed by Dunnett's post hoc test. Data are mean±s.e.mean.

### SEQUENCE LISTING

Where an initial Met amino acid residue or a corresponding initial codon is indicated in any of the following SEQ ID NOs, said residue/codon is optional.

SEQ ID NO: 1—Nucleotide Sequence of Recombinant Catalytically Inactive BoNT/A (rBoNT/A(0))

SEQ ID NO: 2—Polypeptide Sequence of rBoNT/A(0)

SEQ ID NO: 3—Nucleotide Sequence of rLH_{N}/A (light-chain plus translocation domain only).

SEQ ID NO: 4—Polypeptide Sequence of rLH_{N}/A

SEQ ID NO: 5—Nucleotide Sequence of rL/A (light-chain only)

SEQ ID NO: 6—Polypeptide Sequence of rL/A

SEQ ID NO: 7—Nucleotide Sequence of rH_{C}/A

SEQ ID NO: 8—Polypeptide Sequence of rH_{C}/A

SEQ ID NO: 9—Nucleotide Sequence of rBoNT/B(0)

SEQ ID NO: 10—Polypeptide Sequence of rBoNT/B(0)

SEQ ID NO: 11—Nucleotide Sequence of rBoNT/C(0)

SEQ ID NO: 12—Polypeptide Sequence of rBoNT/C(0)

SEQ ID NO: 13—Nucleotide Sequence of rBoNT/E(0)

SEQ ID NO: 14—Polypeptide Sequence of rBoNT/E(0)

SEQ ID NO: 15—Nucleotide Sequence of rBoNT/F(0)

SEQ ID NO: 16—Polypeptide Sequence of rBoNT/F(0)

SEQ ID NO: 17—Nucleotide Sequence of rBoNT/A(0) (His-tagged)

SEQ ID NO: 18—Polypeptide Sequence of rBoNT/A(0) (His-tagged)

SEQ ID NO: 19—Nucleotide Sequence of rLH_{N}/A (His-tagged)

SEQ ID NO: 20—Polypeptide Sequence of rLH_{N}/A (His-tagged)

SEQ ID NO: 21—Nucleotide Sequence of rH_{C}/A (His-tagged)

SEQ ID NO: 22—Polypeptide Sequence of rH_{C}/A (His-tagged)

SEQ ID NO: 23—Nucleotide Sequence of rLC/A (His-tagged)

SEQ ID NO: 24—Polypeptide Sequence of rLC/A (His-tagged)

SEQ ID NO: 25—Nucleotide Sequence of rBoNT/FA(0) (His-tagged)

SEQ ID NO: 26—Polypeptide Sequence of rBoNT/FA(0) (His-tagged)

SEQ ID NO: 27—Nucleotide Sequence of rLH_{N}/FA (His-tagged)

SEQ ID NO: 28—Polypeptide Sequence of rLH_{N}/FA (His-tagged)

SEQ ID NO: 29—Nucleotide Sequence of rH_{C}/FA (His-tagged)

SEQ ID NO: 30—Polypeptide Sequence of rH_{C}/FA (His-tagged)

SEQ ID NO: 31—Nucleotide Sequence of rLC/FA (His-tagged)

SEQ ID NO: 32—Polypeptide Sequence of rLC/FA (His-tagged)

SEQ ID NO: 33—Nucleotide Sequence of rBoNT/F(0) (His-tagged)

SEQ ID NO: 34—Polypeptide Sequence of rBoNT/F(0) (His-tagged)

SEQ ID NO: 35—Nucleotide Sequence of rL_{H}N/F (His-tagged)

SEQ ID NO: 36—Polypeptide Sequence of rL_{H}N/F (His-tagged)

SEQ ID NO: 37—Nucleotide Sequence of rH_{C}/F (His-tagged)

SEQ ID NO: 38—Polypeptide Sequence of rH_{C}/F (His-tagged)

SEQ ID NO: 39—Nucleotide Sequence of rLC/F (His-tagged)

SEQ ID NO: 40—Polypeptide Sequence of rLC/F (His-tagged)

SEQ ID NO: 41—Nucleotide Sequence of Cationic rH_{C}/A (His-tagged)

SEQ ID NO: 42—Polypeptide Sequence of Cationic rH_{C}/A (His-tagged)

SEQ ID NO: 43—Nucleotide Sequence of rH_{C}/AB (His-tagged)

SEQ ID NO: 44—Polypeptide Sequence of rH_{C}/AB (His-tagged)

SEQ ID NO: 45—Nucleotide Sequence of rH_{C}/A Variant Y1117V H1253K (His-tagged)

SEQ ID NO: 46—Polypeptide Sequence of rH_{C}/A Variant Y1117V H1253K (His-tagged)

SEQ ID NO: 47—Nucleotide Sequence of rH_{C}/A Variant Y1117V F1252Y H1253K L1278F (His-tagged)

SEQ ID NO: 48—Polypeptide Sequence of rH_{C}/A Variant Y1117V F1252Y H1253K L1278F (His-tagged)

SEQ ID NO: 49—Nucleotide Sequence of rH_{C}/A Variant Y1117V F1252Y H1253K L1278H (His-tagged)

SEQ ID NO: 50—Polypeptide Sequence of rH_{C}/A Variant Y1117V F1252Y H1253K L1278H (His-tagged)

SEQ ID NO: 51—Polypeptide Sequence of BoNT/A—UniProt P10845

SEQ ID NO: 52—Polypeptide Sequence of BoNT/B—UniProt P10844

SEQ ID NO: 53—Polypeptide Sequence of BoNT/C—UniProt P18640

SEQ ID NO: 54—Polypeptide Sequence of BoNT/D—UniProt P19321

SEQ ID NO: 55—Polypeptide Sequence of BoNT/E—UniProt Q00496

SEQ ID NO: 56—Polypeptide Sequence of BoNT/F—UniProt A7GBG3

SEQ ID NO: 57—Polypeptide Sequence of BoNT/G—UniProt Q60393

SEQ ID NO: 58—Polypeptide Sequence of TeNT— UniProt P04958

SEQ ID NO: 59—Polypeptide Sequence of BoNT/X

SEQ ID NO: 60—Nucleotide Sequence of mrBoNT/A

SEQ ID NO: 61—Polypeptide Sequence of mrBoNT/A

SEQ ID NO: 62—Polypeptide Sequence of Unmodified BoNT/A1

SEQ ID NO: 63—Polypeptide Sequence of mrBoNT/AB

SEQ ID NO: 64—Polypeptide Sequence of mrBoNT/AB(0)

SEQ ID NO: 65—Polypeptide Sequence of mrBoNT/A(0)

Nucleotide Sequence of rBoNT/A(0)
SEQ ID NO: 1
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATcAACTG
ATCtACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
Polypeptide Sequence of rBoNT/A(0)
SEQ ID NO: 2
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
Nucleotide Sequence of rLH_{N}/A
SEQ ID NO: 3
atggagttcgttaacaaacagttcaactataaagacccagttaacggtgttgacattgcttacatcaaaatcccg
aacgctggccagatgcagccggtaaaggcattcaaaatccacaacaaaatctgggttatcccggaacgtgatacc
tttactaacccggaagaaggtgacctgaacccgccaccggaagcgaaacaggtgccggtatcttactatgactcc
acctacctgtctaccgataacgaaaaggacaactacctgaaaggtgttactaaactgttcgagcgtatttactcc
accgacctgggccgtatgctgctgactagcatcgttcgcggtatcccgttctggggcggttctaccatcgatacc
gaactgaaagtaatcgacactaactgcatcaacgttattcagccggacggttcctatcgttccgaagaactgaac
ctggtgatcatcggcccgtctgctgatatcatccagttcgagtgtaagagctttggtcacgaagttctgaacctc
acccgtaacggctacggttccactcagtacatccgtttctctccggacttcaccttcggttttgaagaatccctg
gaagtagacacgaacccactgctgggcgctggtaaattcgcaactgatcctgcggttaccctggctcacgaactg
attcatgcaggccaccgcctgtacggtatcgccatcaatccgaaccgtgtcttcaaagttaacaccaacgcgtat
tacgagatgtccggtctggaagttagcttcgaagaactgcgtacttttggcggtcacgacgctaaattcatcgac
tctctgcaagaaaacgagttccgtctgtactactataacaagttcaaagatatcgcatccaccctgaacaaagcg
aaatccatcgtgggtaccactgcttctctccagtacatgaagaacgtttttaaagaaaaatacctgctcagcgaa
gacacctccggcaaattctctgtagacaagttgaaattcgataaactttacaaaatgctgactgaaatttacacc
gaagacaacttcgttaagttctttaaagttctgaaccgcaaaacctatctgaacttcgacaaggcagtattcaaa
atcaacatcgtgccgaaagttaactacactatctacgatggtttcaacctgcgtaacaccaacctggctgctaat
tttaacggccagaacacggaaatcaacaacatgaacttcacaaaactgaaaaacttcactggtctgttcgagttt
tacaagctgctgtgcGTCGACGGCATCATTACCTCCAAAACTAAATCTGACGATGACGATAAAAACAAAGCGCTG
AACCTGCAGtgtatcaaggttaacaactgggatttattcttcagcccgagtgaagacaacttcaccaacgacctg
aacaaaggtgaagaaatcacctcagatactaacatcgaagcagccgaagaaaacatctcgctggacctgatccag
cagtactacctgacctttaatttcgacaacgagccggaaaacatttctatcgaaaacctgagctctgatatcatc
ggccagctggaactgatgccgaacatcgaacgtttcccaaacggtaaaaagtacgagctggacaaatataccatg
ttccactacctgcgcgcgcaggaatttgaacacggcaaatcccgtatcgcactgactaactccgttaacgaagct
ctgctcaacccgtcccgtgtatacaccttcttctctagcgactacgtgaaaaaggtcaacaaagcgactgaagct
gcaatgttcttgggttgggttgaacagcttgtttatgattttaccgacgagacgtccgaagtatctactaccgac
aaaattgcggatatcactatcatcatcccgtacatcggtccggctctgaacattggcaacatgctgtacaaagac
gacttcgttggcgcactgatcttctccggtgcggtgatcctgctggagttcatcccggaaatcgccatcccggta
ctgggcacctttgctctggtttcttacattgcaaacaaggttctgactgtacaaaccatcgacaacgcgctgagc
aaacgtaacgaaaaatgggatgaagtttacaaatatatcgtgaccaactggctggctaaggttaatactcagatc
gacctcatccgcaaaaaaatgaaagaagcactggaaaaccaggcggaagctaccaaggcaatcattaactaccag
tacaaccagtacaccgaggaagaaaaaaacaacatcaacttcaacatcgacgatctgtcctctaaactgaacgaa
tccatcaacaaagctatgatcaacatcaacaagttcctgaaccagtgctctgtaagctatctgatgaactccatg
atcccgtacggtgttaaacgtctggaggacttcgatgcgtctctgaaagacgccctgctgaaatacatttacgac
aaccgtggcactctgatcggtcaggttgatcgtctgaaggacaaagtgaacaataccttatcgaccgacatccct
tttcagctcagtaaatatgtcgataaccaacgccttttgtccactctagaagcaCACCATCATCACcaccatcac
catcaccat
Polypeptide Sequence of rLH_{N}/A
SEQ ID NO: 4
MEFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVDGIITSKTKSDDDDKNKAL
NLQCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTLEAHHHHHHHHHH
Nucleotide Sequence of rL/A
SEQ ID NO: 5
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGggtctagaagcaCACCATCATCACcaccatcaccatcaccat
Polypeptide Sequence of rL/A
SEQ ID NO: 6
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLGLEAHHHHHHHHHH
Nucleotide Sequence of rH_{C}/A
SEQ ID NO: 7
ATGCATCATCACCATCACCACAAAAACATCATCAATACTAGCATTCTGAACCTGCGTTACGAGAGCAATCATCTG
ATTGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTAC
GAGAATTTCTCCACCAGCTTCTGGATTCGCATCCCGAAATACTTCAACAGCATTAGCCTGAACAACGAGTATACT
ATCATCAACTGTATGGAGAACAACAGCGGTTGGAAGGTGTCTCTGAACTATGGTGAGATCATTTGGACCTTGCAG
GACACCCAAGAGATCAAGCAGCGCGTCGTGTTCAAGTACTCTCAAATGATCAACATTTCCGATTACATTAATCGT
TGGATCTTCGTGACCATTACGAATAACCGTCTGAATAACAGCAAGATTTACATCAATGGTCGCTTGATCGATCAG
AAACCGATTAGCAACCTGGGTAATATCCACGCAAGCAACAACATTATGTTCAAATTGGACGGTTGCCGCGATACC
CATCGTTATATCTGGATCAAGTATTTCAACCTGTTTGATAAAGAACTGAATGAGAAGGAGATCAAAGATTTGTAT
GACAACCAATCTAACAGCGGCATTTTGAAGGACTTCTGGGGCGATTATCTGCAATACGATAAGCCGTACTATATG
CTGAACCTGTATGATCCGAACAAATATGTGGATGTCAATAATGTGGGTATTCGTGGTTACATGTATTTGAAGGGT
CCGCGTGGCAGCGTTATGACGACCAACATTTACCTGAACTCTAGCCTGTACCGTGGTACGAAATTCATCATTAAG
AAATATGCCAGCGGCAACAAAGATAACATTGTGCGTAATAACGATCGTGTCTACATCAACGTGGTCGTGAAGAAT
AAAGAGTACCGTCTGGCGACCAACGCTTCGCAGGCGGGTGTTGAGAAAATTCTGAGCGCGTTGGAGATCCCTGAT
GTCGGTAATCTGAGCCAAGTCGTGGTTATGAAGAGCAAGAACGACCAGGGTATCACTAACAAGTGCAAGATGAAC
CTGCAAGACAACAATGGTAACGACATCGGCTTTATTGGTTTCCACCAGTTCAACAATATTGCTAAACTGGTAGCG
AGCAATTGGTACAATCGTCAGATTGAGCGCAGCAGCCGTACTTTGGGCTGTAGCTGGGAGTTTATCCCGGTCGAT
GATGGTTGGGGCGAACGTCCGCTG
Polypeptide Sequence of rH_{C}/A
SEQ ID NO: 8
MHHHHHHKNIINTSILNLRYESNHLIDLSRYASKINIGSKVNFDPIDKNQIQLFNLESSKIEVILKNAIVYNSMY
ENFSTSFWIRIPKYFNSISLNNEYTIINCMENNSGWKVSLNYGEIIWTLQDTQEIKQRVVFKYSQMINISDYINR
WIFVTITNNRLNNSKIYINGRLIDQKPISNLGNIHASNNIMFKLDGCRDTHRYIWIKYFNLFDKELNEKEIKDLY
DNQSNSGILKDFWGDYLQYDKPYYMLNLYDPNKYVDVNNVGIRGYMYLKGPRGSVMTTNIYLNSSLYRGTKFIIK
KYASGNKDNIVRNNDRVYINVVVKNKEYRLATNASQAGVEKILSALEIPDVGNLSQVVVMKSKNDQGITNKCKMN
LQDNNGNDIGFIGFHQFNNIAKLVASNWYNRQIERSSRTLGCSWEFIPVDDGWGERPL
Nucleotide Sequence of rBoNT/B(0)
SEQ ID NO: 9
ATGCCGGTGACGATTAACAACTTCAACTACAACGACCCGATTGACAACAACAACATTATCATGATGGAACCGCCG
TTTGCACGCGGCACGGGCCGTTATTACAAAGCGTTTAAAATCACCGATCGTATTTGGATTATCCCGGAACGCTAC
ACGTTTGGTTATAAACCGGAAGACTTCAACAAAAGCTCTGGCATCTTCAACCGTGATGTTTGCGAATACTACGAT
CCGGACTACCTGAACACCAACGATAAGAAAAACATTTTTCTGCAAACGATGATCAAACTGTTCAATCGCATTAAA
AGCAAACCGCTGGGTGAAAAACTGCTGGAAATGATTATCAATGGCATTCCGTATCTGGGTGATCGTCGCGTGCCG
CTGGAAGAATTTAACACCAATATCGCGAGTGTTACGGTCAACAAACTGATTTCCAATCCGGGTGAAGTCGAACGT
AAAAAAGGCATCTTCGCCAACCTGATCATCTTCGGCCCGGGTCCGGTGCTGAACGAAAATGAAACCATTGATATC
GGTATTCAGAACCATTTTGCCTCACGCGAAGGCTTCGGCGGTATTATGCAAATGAAATTTTGCCCGGAATATGTG
TCGGTTTTCAACAATGTTCAGGAAAACAAAGGTGCAAGCATCTTTAATCGTCGCGGCTATTTCTCTGATCCGGCT
CTGATCCTGATGCACcAACTGATTtATGTGCTGCACGGCCTGTATGGTATCAAAGTGGATGACCTGCCGATCGTT
CCGAACGAGAAAAAATTTTTCATGCAGAGCACCGACGCAATTCAAGCTGAAGAACTGTATACGTTTGGCGGTCAG
GACCCGTCTATTATCACCCCGAGCACCGACAAAAGCATCTACGATAAAGTGCTGCAAAACTTTCGTGGCATTGTT
GACCGCCTGAATAAAGTCCTGGTGTGTATCTCTGATCCGAACATCAACATCAACATCTACAAAAACAAATTCAAA
GACAAATACAAATTCGTTGAAGATTCTGAAGGCAAATATAGTATTGACGTCGAATCCTTTGATAAACTGTACAAA
AGTCTGATGTTCGGTTTCACCGAAACGAACATCGCGGAAAACTACAAAATCAAAACCCGCGCCTCCTATTTCAGC
GACTCTCTGCCGCCGGTTAAAATCAAAAATCTGCTGGATAACGAAATTTATACGATCGAAGAAGGTTTCAACATC
AGCGATAAAGACATGGAAAAAGAATACCGTGGCCAGAATAAAGCAATCAACAAACAGGCGTATGAAGAAATTAGT
AAAGAACATCTGGCGGTCTACAAAATTCAGATGTGCAAATCCGTGAAAGCCCCGGGTATTTGTATCGATGTTGAC
AATGAAGACCTGTTTTTCATCGCCGATAAAAACAGTTTTTCCGATGACCTGTCAAAAAATGAACGCATCGAATAC
AACACCCAATCGAACTACATCGAAAACGATTTCCCGATCAACGAACTGATTCTGGATACGGACCTGATTAGTAAA
ATCGAACTGCCGTCAGAAAACACCGAATCGCTGACGGACTTTAATGTTGATGTCCCGGTGTATGAAAAACAGCCG
GCAATTAAGAAAATTTTTACCGATGAAAACACGATCTTCCAGTACCTGTACAGCCAAACCTTTCCGCTGGACATT
CGCGATATCTCTCTGACGAGTTCCTTTGATGACGCACTGCTGTTCAGCAACAAAGTGTACTCCTTTTTCTCAATG
GATTACATCAAAACCGCTAACAAAGTGGTTGAAGCGGGCCTGTTTGCCGGTTGGGTGAAACAGATCGTTAACGAT
TTCGTCATCGAAGCCAACAAAAGTAACACGATGGATAAAATTGCTGATATCTCCCTGATTGTCCCGTATATTGGC
CTGGCACTGAATGTGGGTAACGAAACGGCGAAAGGCAATTTTGAAAACGCCTTCGAAATTGCAGGCGCTTCAATC
CTGCTGGAATTTATTCCGGAACTGCTGATCCCGGTCGTGGGTGCGTTCCTGCTGGAATCTTACATCGACAACAAA
AACAAAATCATCAAAACCATTGATAACGCGCTGACGAAACGTAACGAAAAATGGTCAGATATGTACGGCCTGATT
GTTGCCCAGTGGCTGAGCACCGTCAACACGCAATTTTACACCATCAAAGAAGGTATGTACAAAGCGCTGAATTAT
CAGGCGCAAGCCCTGGAAGAAATCATCAAATACCGCTACAACATCTACAGCGAAAAAGAAAAATCTAACATCAAC
ATCGACTTTAATGATATCAACAGCAAACTGAACGAAGGTATCAACCAGGCAATCGATAACATCAACAACTTCATC
AACGGCTGCTCAGTGTCGTATCTGATGAAGAAAATGATCCCGCTGGCTGTTGAAAAACTGCTGGATTTTGACAAC
ACCCTGAAGAAAAACCTGCTGAACTACATCGATGAAAACAAACTGTACCTGATCGGCTCAGCCGAATACGAAAAA
TCGAAAGTGAACAAATACCTGAAAACCATCATGCCGTTTGACCTGAGTATTTACACCAACGATACGATCCTGATC
GAAATGTTCAACAAATACAACTCCGAAATTCTGAACAATATTATCCTGAACCTGCGTTACAAAGACAACAATCTG
ATCGATCTGAGCGGCTATGGTGCAAAAGTTGAAGTCTACGACGGTGTCGAACTGAACGATAAAAACCAGTTCAAA
CTGACCTCATCGGCTAACTCAAAAATTCGTGTGACGCAGAACCAAAACATCATCTTCAACTCGGTCTTTCTGGAC
TTCAGCGTGTCTTTCTGGATTCGCATCCCGAAATATAAAAATGATGGCATCCAGAACTACATCCATAACGAATAC
ACCATCATCAACTGTATGAAAAACAACAGTGGTTGGAAAATTTCCATCCGTGGCAACCGCATTATCTGGACCCTG
ATTGATATCAATGGTAAAACGAAAAGCGTGTTTTTCGAATACAACATCCGTGAAGATATCTCTGAATACATCAAT
CGCTGGTTTTTCGTGACCATTACGAACAATCTGAACAATGCGAAAATCTATATCAACGGCAAACTGGAAAGTAAT
ACCGACATCAAAGATATTCGTGAAGTTATCGCCAACGGTGAAATCATCTTCAAACTGGATGGCGACATCGATCGC
ACCCAGTTCATTTGGATGAAATACTTCTCCATCTTCAACACGGAACTGAGTCAGTCCAATATCGAAGAACGCTAC
AAAATCCAATCATACTCGGAATACCTGAAAGATTTCTGGGGTAACCCGCTGATGTACAACAAAGAATACTACATG
TTCAACGCGGGCAACAAAAACTCATACATCAAACTGAAAAAAGATTCGCCGGTGGGTGAAATCCTGACCCGTAGC
AAATACAACCAGAACTCTAAATACATCAACTATCGCGATCTGTACATTGGCGAAAAATTTATTATCCGTCGCAAA
AGCAACTCTCAGAGTATTAATGATGACATCGTGCGTAAAGAAGACTACATCTATCTGGATTTCTTTAATCTGAAC
CAAGAATGGCGCGTTTATACCTACAAATACTTCAAAAAAGAAGAAGAGAAACTGTTCCTGGCCCCGATTAGCGAC
AGCGATGAATTTTACAACACCATCCAGATCAAAGAATACGATGAACAGCCGACGTATAGTTGCCAACTGCTGTTC
AAAAAAGACGAAGAATCCACCGATGAAATTGGCCTGATTGGTATCCACCGTTTCTATGAAAGCGGTATCGTTTTC
GAAGAATACAAAGATTACTTCTGTATCTCTAAATGGTATCTGAAAGAAGTCAAACGCAAACCGTACAACCTGAAA
CTGGGCTGCAACTGGCAATTTATCCCGAAAGACGAAGGCTGGACCGAA
Polypeptide Sequence of rBoNT/B(0)
SEQ ID NO: 10
MPVTINNFNYNDPIDNNNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVFNNVQENKGASIFNRRGYFSDPA
LILMHQLIYVLHGLYGIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGFNISDKDMEKEYRGQNKAINKQAYEEISKEHLAVYKIQMCKSVKAPGICIDVD
NEDLFFIADKNSFSDDLSKNERIEYNTQSNYIENDFPINELILDTDLISKIELPSENTESLTDFNVDVPVYEKQP
AIKKIFTDENTIFQYLYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLIPVVGAFLLESYIDNK
NKIIKTIDNALTKRNEKWSDMYGLIVAQWLSTVNTQFYTIKEGMYKALNYQAQALEEIIKYRYNIYSEKEKSNIN
IDFNDINSKLNEGINQAIDNINNFINGCSVSYLMKKMIPLAVEKLLDFDNTLKKNLLNYIDENKLYLIGSAEYEK
SKVNKYLKTIMPFDLSIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEIIFKLDGDIDR
TQFIWMKYFSIFNTELSQSNIEERYKIQSYSEYLKDFWGNPLMYNKEYYMFNAGNKNSYIKLKKDSPVGEILTRS
KYNQNSKYINYRDLYIGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCISKWYLKEVKRKPYNLK
LGCNWQFIPKDEGWTE
Nucleotide Sequence of rBoNT/C(0)
SEQ ID NO: 11
ATGCCGATCACGATTAATAATTTCAACTATAGCGATCCGGTGGACAATAAGAATATTCTGTATCTGGATACTCAT
CTGAATACGCTGGCTAACGAACCGGAGAAAGCGTTCCGCATCACAGGCAACATCTGGGTTATTCCCGATCGCTTT
TCACGCAACAGCAACCCTAATCTGAACAAACCTCCTCGTGTCACCAGTCCTAAATCCGGTTATTACGACCCAAAC
TATCTGAGTACGGATAGCGATAAAGATCCCTTTCTGAAAGAGATCATTAAGCTGTTCAAACGCATTAACTCTCGC
GAAATTGGGGAAGAGCTGATCTATCGGCTTTCGACAGATATCCCGTTCCCAGGTAACAATAATACCCCGATTAAT
ACTTTCGACTTTGATGTTGATTTCAATTCTGTGGATGTGAAAACGCGTCAAGGCAATAATTGGGTGAAAACTGGT
AGCATTAACCCGAGTGTAATTATCACAGGTCCCCGTGAGAACATCATCGACCCGGAAACCTCTACCTTCAAGCTG
ACGAACAACACGTTTGCTGCACAGGAAGGGTTTGGTGCCCTGTCAATCATTTCCATCTCACCGCGTTTCATGTTA
ACCTACTCCAATGCCACAAATGATGTTGGCGAAGGACGTTTTAGCAAATCAGAATTTTGCATGGACCCAATTCTC
ATTCTGATGggCacGCTGAACaATGCGATGCACAACTTGTATGGCATTGCTATTCCAAACGATCAAACCATTAGC
TCCGTTACCAGTAATATCTTCTATAGCCAGTATAATGTCAAATTGGAGTATGCCGAAATTTACGCCTTTGGAGGC
CCGACCATTGACCTGATTCCGAAATCTGCACGCAAATACTTCGAAGAAAAGGCGTTAGATTACTATCGCAGCATC
GCGAAACGCCTGAACTCGATTACCACGGCCAATCCGTCGTCGTTCAACAAATACATTGGTGAATATAAACAGAAA
CTGATTCGCAAATATCGGTTTGTCGTAGAAAGCTCTGGTGAAGTGACTGTAAACCGCAACAAATTTGTCGAACTC
TACAACGAGTTGACCCAAATCTTTACCGAGTTTAACTACGCAAAGATCTATAACGTACAGAACCGCAAGATTTAT
CTTAGCAATGTATACACACCGGTTACTGCGAACATCTTAGACGACAATGTGTATGATATTCAGAATGGCTTTAAC
ATCCCGAAATCAAATCTGAACGTTCTGTTTATGGGCCAGAACCTGAGTCGTAATCCAGCACTGCGTAAAGTGAAC
CCGGAAAATATGCTCTACTTGTTTACCAAATTTTGCCACAAAGCGATTGATGGCCGCTCTCTCTATAACAAAACG
CTGGATTGTCGTGAGTTACTTGTGAAGAACACTGATTTACCGTTCATTGGGGATATCTCCGACGTGAAAACCGAT
ATCTTCCTGCGCAAAGACATTAATGAAGAAACGGAAGTCATCTATTACCCCGACAATGTGAGCGTTGATCAGGTC
ATTTTATCGAAGAACACCTCCGAACATGGTCAGTTGGATTTGCTGTACCCTAGCATTGACTCGGAGAGTGAAATC
CTTCCGGGCGAAAATCAAGTGTTTTACGACAACCGTACCCAAAATGTTGATTATTTGAATTCTTATTACTACCTG
GAATCTCAGAAATTGAGCGACAATGTGGAAGATTTCACGTTCACACGCTCCATTGAGGAAGCGCTGGATAATAGC
GCGAAAGTGTATACGTATTTCCCTACCTTGGCGAATAAAGTAAATGCTGGTGTCCAGGGAGGCTTATTTCTGATG
TGGGCGAATGATGTGGTAGAAGATTTTACGACCAATATTTTGCGTAAGGACACCTTAGATAAAATTAGCGATGTT
AGCGCCATCATCCCCTATATTGGCCCAGCACTGAATATCTCGAACTCTGTGCGTCGCGGAAACTTCACCGAAGCA
TTTGCGGTGACCGGGGTTACTATTCTGTTGGAAGCCTTTCCGGAGTTTACTATTCCGGCGCTGGGTGCGTTTGTG
ATTTATTCGAAAGTACAAGAACGCAATGAAATTATCAAAACCATCGATAATTGCCTGGAACAACGCATTAAACGC
TGGAAGGATTCTTATGAATGGATGATGGGCACCTGGTTATCCCGTATTATCACACAGTTTAACAACATCTCGTAT
CAGATGTACGATTCACTGAACTACCAAGCAGGGGCGATCAAAGCCAAGATCGACTTAGAATACAAGAAATATTCA
GGTAGCGATAAAGAGAATATTAAAAGCCAGGTTGAAAACCTGAAGAACTCTCTGGATGTCAAAATTTCAGAGGCT
ATGAACAACATTAACAAATTTATCCGCGAATGTAGCGTCACGTATCTGTTTAAAAACATGCTCCCGAAAGTGATT
GATGAGCTCAACGAGTTTGATCGCAACACAAAGGCCAAACTGATTAACCTGATTGATAGTCACAATATTATTTTA
GTCGGTGAAGTTGACAAGCTGAAGGCTAAGGTCAATAACAGCTTTCAGAACACTATTCCGTTTAATATTTTCTCC
TATACGAACAATAGTCTGCTGAAAGACATTATCAACGAATACTTCAACAATATTAATGACAGCAAAATTCTGAGC
CTGCAGAATCGTAAGAATACGCTGGTAGATACCAGTGGATATAATGCGGAAGTCTCAGAAGAGGGTGATGTACAG
CTGAACCCGATCTTTCCGTTCGACTTTAAACTGGGGTCTAGTGGTGAAGATCGCGGTAAAGTGATCGTTACCCAA
AACGAGAACATTGTGTATAACAGCATGTACGAGAGTTTCTCAATTTCTTTCTGGATTCGCATCAATAAATGGGTT
TCTAATTTGCCTGGCTATACCATCATTGATAGCGTCAAAAACAACTCGGGCTGGTCGATTGGCATTATTAGCAAC
TTTCTGGTGTTTACCCTGAAACAGAATGAGGATTCGGAACAGAGCATTAACTTCTCCTACGACATCAGCAACAAT
GCACCAGGGTATAACAAATGGTTCTTCGTAACGGTGACGAACAATATGATGGGCAATATGAAAATCTACATTAAC
GGGAAACTTATCGACACCATTAAAGTGAAAGAGCTTACTGGGATCAATTTTAGTAAAACCATTACCTTTGAGATC
AACAAAATTCCGGACACGGGTCTGATTACCTCCGATTCGGATAATATCAATATGTGGATTCGCGACTTTTATATC
TTCGCCAAAGAACTTGATGGCAAAGATATCAACATTTTGTTTAATTCCCTGCAGTATACCAATGTCGTTAAGGAC
TATTGGGGCAATGATCTCCGCTACAATAAAGAATACTACATGGTTAACATCGACTATCTCAATCGCTACATGTAT
GCTAACTCGCGTCAAATTGTGTTTAACACACGTCGTAACAACAACGATTTTAACGAAGGTTATAAAATCATTATC
AAACGGATCCGCGGCAATACGAACGATACTCGTGTTCGTGGCGGTGACATTCTGTATTTCGACATGACGATTAAT
AATAAAGCGTACAATCTGTTCATGAAGAACGAAACCATGTACGCCGATAACCATTCCACTGAAGATATCTACGCA
ATCGGACTTCGCGAACAGACCAAAGACATTAACGACAACATCATCTTTCAGATTCAACCGATGAATAATACCTAC
TACTATGCCTCCCAGATCTTCAAAAGTAATTTCAACGGCGAAAACATTTCAGGCATTTGCTCAATCGGCACTTAT
CGGTTCCGGTTAGGTGGTGATTGGTATCGTCACAACTACCTTGTTCCCACAGTGAAACAAGGCAACTATGCATCG
CTCTTAGAAAGCACATCTACGCATTGGGGTTTTGTGCCAGTCAGTGAA
Polypeptide Sequence of rBoNT/C(0)
SEQ ID NO: 12
MPITINNFNYSDPVDNKNILYLDTHLNTLANEPEKAFRITGNIWVIPDRFSRNSNPNLNKPPRVTSPKSGYYDPN
YLSTDSDKDPFLKEIIKLFKRINSREIGEELIYRLSTDIPFPGNNNTPINTFDFDVDFNSVDVKTRQGNNWVKTG
SINPSVIITGPRENIIDPETSTFKLTNNTFAAQEGFGALSIISISPRFMLTYSNATNDVGEGRFSKSEFCMDPIL
ILMGTLNNAMHNLYGIAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTEFNYAKIYNVQNRKIY
LSNVYTPVTANILDDNVYDIQNGFNIPKSNLNVLFMGQNLSRNPALRKVNPENMLYLFTKFCHKAIDGRSLYNKT
LDCRELLVKNTDLPFIGDISDVKTDIFLRKDINEETEVIYYPDNVSVDQVILSKNTSEHGQLDLLYPSIDSESEI
LPGENQVFYDNRTQNVDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILLEAFPEFTIPALGAFV
IYSKVQERNEIIKTIDNCLEQRIKRWKDSYEWMMGTWLSRIITQFNNISYQMYDSLNYQAGAIKAKIDLEYKKYS
GSDKENIKSQVENLKNSLDVKISEAMNNINKFIRECSVTYLFKNMLPKVIDELNEFDRNTKAKLINLIDSHNIIL
VGEVDKLKAKVNNSFQNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIIDSVKNNSGWSIGIISN
FLVFTLKQNEDSEQSINFSYDISNNAPGYNKWFFVTVTNNMMGNMKIYINGKLIDTIKVKELTGINFSKTITFEI
NKIPDTGLITSDSDNINMWIRDFYIFAKELDGKDINILFNSLQYTNVVKDYWGNDLRYNKEYYMVNIDYLNRYMY
ANSRQIVFNTRRNNNDFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYRHNYLVPTVKQGNYAS
LLESTSTHWGFVPVSE
Nucleotide Sequence of rBoNT/E(0)
SEQ ID NO: 13
atgccgaaaatcaactctttcaactacaacgacccggttaacgaccgtaccatcctgtatatcaaaccgggtggt
tgccaggagttctacaaatctttcaacatcatgaaaaacatctggatcatcccggaacgtaacgttatcggtacc
accccgcaggacttccacccgccgacctctctgaaaaacggtgactcttcttactacgacccgaactacctccag
tctgacgaagaaaaagaccgtttcctgaaaatcgttaccaaaatcttcaaccgtatcaacaacaacctgtctggt
ggtatcctgctggaagaactgtctaaagctaacccgtacctgggtaacgacaacaccccggacaaccagttccac
atcggtgacgcttctgctgttgaaatcaaattctctaacggttctcaggacatcctgctgccgaacgttatcatc
atgggtgctgaaccggacctgttcgaaaccaactcttctaacatctctctgcgtaacaactacatgccgtctaac
cacggtttcggttctatcgctatcgttaccttctctccggaatactctttccgtttcaacgacaacagcatgaac
gagttcatccaggacccggctctgaccctgatgcaccaactgatctactctctgcacggtctgtacggtgctaaa
ggtatcaccaccaaatacaccatcacccagaaacagaacccgctgatcaccaacatccgtggtaccaacatcgaa
gagttcctgaccttcggtggtaccgacctgaacatcatcacctctgctcagtctaacgacatctacaccaacctg
ctggctgactacaaaaaaatcgcttctaaactgtctaaagttcaggtttctaacccgctgctgaacccgtacaaa
gacgttttcgaagctaaatacggtctggacaaagacgcttctggtatctactctgttaacatcaacaaattcaac
gacatcttcaaaaaactgtactctttcaccgagttcgacctggcgaccaaattccaggttaaatgccgtcagacc
tacatcggtcagtacaaatacttcaaactgtctaacctgctgaacgactctatctacaacatctctgaaggttac
aacatcaacaacctgaaagttaacttccgtggtcagaacgctaacctgaacccgcgtatcatcaccccgatcacc
ggtcgtggtctggttaaaaaaatcatccgtttctgcAAGAATATTGTAAGCGTTAAAGGAATAAGAAAAAGTATC
tgcatcgaaatcaacaacggtgaactgttcttcgttgcttctgaaaactcttacaacgacgacaacatcaacacc
ccgaaagaaatcgacgacaccgttacctctaacaacaactacgaaaacgacctggaccaggttatcctgaacttc
aactctgaatctgctccgggtctgtctgacgaaaaactgaacctgaccatccagaacgacgcttacatcccgaaa
tacgactctaacggtacctctgacatcgaacagcacgacgttaacgaactgaacgttttcttctacctggacgct
cagaaagttccggaaggtgaaaacaacgttaacctgacctcttctatcgacaccgctctgctggaacagccgaaa
atctacaccttcttctcttctgagttcatcaacaacgttaacaaaccggttcaggctgctctgttcgtttcttgg
attcagcaggttctggttgacttcaccaccgaagctaaccagaaatctaccgttgacaaaatcgctgacatctct
atcgttgttccgtacatcggtctggctctgaacatcggtaacgaagctcagaaaggtaacttcaaagacgctctg
gaactgctgggtgctggtatcctgctggagttcgaaccggaactgctgatcccgaccatcctggttttcaccatc
aaatctttcctgggttcttctgacaacaaaaacaaagttatcaaagctatcaacaacgctctgaaagaacgtgac
gaaaaatggaaagaagtttactctttcatcgtttctaactggatgaccaaaatcaacacccagttcaacaaacgt
aaagaacagatgtaccaggctctccagaaccaggttaacgctatcaaaaccatcatcgaatctaaatacaactct
tacaccctggaagaaaaaaacgaactgaccaacaaatacgacatcaaacagatcgaaaacgaactgaaccagaaa
gtttctatcgctatgaacaacatcgaccgtttcctgaccgaatcttctatctcttacctgatgaaactcatcaac
gaagttaaaatcaacaaactgcgtgaatacgacgaaaacgttaaaacctacctgctgaactacatcatccagcac
ggttctatcctgggtgaatctcagcaggaactgaactctatggttaccgacaccctgaacaactctatcccgttc
aaactgtcttcttacaccgacgacaaaatcctGATCTCTTACTTCAACAAATTCTTTAAAcgcATTAAGAGTTCA
TCGGTTctgaatATGCGGTACAAAAATGATAAAtatGTCGATACTTCTGGATATgatAGCAATATCAACATTAAC
GGCGACGTGTATAAATATccgACAAATAAAAACCAGTTTGGGATATATAACGACAAGctgTCGGAGGTCAATatt
TCTCAAAACGACtatATCattTACGATAATaaaTATAAAAACTTTAGCATTAGTtttTGGGTTcgtATACCTAAT
tatGACAATaaaattGTAAATGTGAATAACGAGTATACCATTATAAACTGTATGcgcGACAATAACAGTGGTTGG
AAGGTATCGctgAACCATAATGAGATTATCTGGACCctgcagGATAATgcaGGTATAAACCAGAAACTGGCTTTT
AACTATGGAAACGCAAATGGGATCTCAGATTACATTaataaarGGatttttGTTaccATTACGAACGATcgcTTA
GGCGACTCAAAACTTTATATTAATggcAATctgATAGATCAGAAATCAATCTTAAATTTGGGCAATATTCATGTC
TCTgatAACATCTTGTTCAAGATCGTTAATTGCAGTTACACTcgtTATATTGGCATTCGTTACTTTAATATCTTC
gataaaGAActgGACGAGACGGAAATCcagACTCTGTATTCAAACGAGCCCAATACTAATATATTGAAAGATTTT
TGGGGTAACTATCTTTTATATGATAAAGAATACTATCTCCTGaatGTATTGAAGCCAAACAATTTCATAGATAGA
CGCAAGGATAGCACATTAAGTATCAACAATATCAGATCTACTATActgttaGCAAATCGCCTcTACTCCggtATT
AAAGTGAAGATTcagCGGGTTAATAACTCCAGTACCAATGATAATCTGGTCCGTAAGAACGATCAGGTATACATC
aatTTCGTCGCGAGCAAAACTcatCTCTTCCCGCTTTACGCCgatACAGCTACGACAAACAAGGAAAAAACCATA
AAAATTTCCAGCTCCGGAAACAGATTCAATCAAGTAGTTGTAATGAACTCTGTGGGTaatAATTGTACGATGAAC
TTTaagAATAACAATGGGAACAATattGGACTTTTGGGCTTcAAAGCCGACACAGTGGTGGCGTCCACCTGGTAT
TACACGcacATGcggGACCATACGAATTCGAACGGTTGCTTCTGGAACTTTATCTCGGAAgaaCACGGGTGGCAA
GAAAAA
Polypeptide Sequence of rBoNT/E(0)
SEQ ID NO: 14
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRFNDNSMNEFIQDPALTLMHQLIYSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKFNDIFKKLYSFTEFDLATKFQVKCRQTYIGQYKYFKLSNLLNDSIYNISEGY
NINNLKVNFRGQNANLNPRIITPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
Nucleotide Sequence of rBoNT/F(0)
SEQ ID NO: 15
ATGCCGGTGGTCATCAACAGCTTCAACTACAACGACCCAGTAAACGACGACACGATCCTGTATATGCAAATCCCG
TATGAAGAGAAGAGCAAGAAGTACTATAAGGCCTTTGAAATCATGCGCAATGTGTGGATTATTCCGGAGCGTAAT
ACGATTGGTACTGACCCAAGCGACTTCGATCCACCTGCGTCTTTGGAAAACGGCTCGTCCGCATATTACGACCCG
AATTACCTGACCACCGATGCGGAGAAAGATCGTTATTTGAAAACCACCATCAAGCTGTTCAAACGCATTAACAGC
AATCCGGCAGGTGAGGTCCTGCTGCAAGAGATTAGCTACGCAAAGCCTTATCTGGGTAATGAGCATACGCCTATT
AACGAGTTTCACCCGGTTACCCGCACTACCAGCGTTAACATCAAGTCCTCGACCAACGTGAAGTCTAGCATTATC
CTGAACCTGCTGGTTCTGGGTGCCGGTCCGGACATCTTCGAAAACTCTAGCTACCCGGTGCGTAAACTGATGGAT
AGCGGCGGTGTTTATGACCCGAGCAATGACGGTTTTGGCAGCATCAATATCGTGACGTTTAGCCCGGAGTACGAG
TACACCTTCAATGATATCAGCGGTGGTTACAATTCTTCTACCGAGAGCTTCATCGCCGACCCGGCGATCAGCCTG
GCACACCAACTGATCTATGCATTGCATGGCTTGTACGGTGCCCGTGGTGTGACGTATAAAGAGACTATCAAGGTT
AAGCAGGCACCTCTGATGATTGCGGAAAAGCCGATTCGCCTGGAAGAGTTCCTGACCTTCGGCGGTCAAGATTTG
AACATCATTACCTCGGCCATGAAAGAGAAAATCTATAACAATTTGCTGGCCAACTATGAAAAGATTGCAACGCGC
TTGTCTCGTGTTAACTCCGCTCCGCCGGAATACGACATTAATGAGTACAAAGACTACTTTCAATGGAAATATGGC
CTGGACAAAAATGCGGATGGTTCTTATACCGTGAATGAAAACAAATTCAATGAAATCTACAAGAAACTGTACAGC
TTCACCGAAATCGATCTGGCGAACAAGTTCAAAGTCAAATGTCGTAATACCTACTTCATCAAATATGGCTTCCTG
AAAGTCCCGAACCTGCTGGACGATGACATCTATACCGTCAGCGAAGGCTTCAACATCGGCAATCTGGCCGTGAAT
AATCGTGGTCAGAACATCAAACTGAATCCGAAAATCATTGACTCCATCCCAGACAAGGGCCTGGTTGAGAAAATC
GTGAAGTTCTGCAAAAGCGTTATTCCGCGTAAAGGTACGAAAGCACCGCCTCGCCTGTGCATTCGCGTTAACAAC
CGTGAGTTGTTCTTTGTGGCATCTGAAAGCAGCTACAACGAGAACGACATCAACACCCCTAAAGAAATTGATGAT
ACCACGAACCTGAATAACAATTATCGCAACAATCTGGACGAGGTGATCCTGGATTACAATTCGGAAACCATTCCG
CAAATTAGCAATCAGACGCTGAACACCCTGGTTCAGGACGATAGCTACGTTCCGCGTTACGACTCCAATGGTACT
AGCGAGATTGAAGAACACAACGTAGTGGACTTGAACGTTTTCTTTTATCTGCACGCCCAGAAGGTTCCGGAGGGC
GAAACCAATATTAGCCTGACCAGCTCGATCGACACCGCGCTGTCTGAGGAGAGCCAAGTCTACACCTTTTTCAGC
AGCGAGTTTATCAACACTATTAACAAGCCAGTTCATGCTGCATTGTTTATCTCTTGGATTAACCAGGTGATTCGC
GACTTTACGACGGAGGCGACCCAGAAGTCTACCTTCGACAAAATTGCAGACATCTCCCTGGTCGTCCCATACGTC
GGCCTGGCGTTGAATATTGGCAATGAAGTTCAAAAAGAGAACTTCAAAGAAGCGTTCGAGCTGCTGGGTGCAGGC
ATCCTGCTGGAGTTCGTGCCGGAACTGTTGATCCCGACCATCCTGGTGTTCACCATTAAGAGCTTCATTGGATCC
TCCGAGAATAAGAACAAGATCATCAAGGCGATCAATAACAGCCTGATGGAGCGTGAAACGAAGTGGAAAGAAATC
TATAGCTGGATTGTTAGCAATTGGCTGACTCGTATTAACACGCAATTCAACAAGCGTAAAGAGCAAATGTACCAA
GCCCTGCAAAACCAAGTTGACGCCATCAAAACGGTAATTGAATACAAGTACAACAATTACACGAGCGATGAGCGC
AACCGCCTGGAAAGCGAATACAACATCAACAACATTCGCGAAGAATTGAACAAGAAAGTGAGCCTGGCGATGGAG
AACATTGAGCGTTTTATCACCGAAAGCAGCATCTTTTACCTGATGAAATTGATTAATGAGGCGAAAGTCTCGAAA
CTGCGTGAGTACGACGAAGGTGTGAAAGAGTATCTGCTGGATTACATTAGCGAGCACCGTAGCATCTTGGGTAAC
TCGGTTCAGGAGCTGAACGATCTGGTGACCTCTACCCTGAACAATAGCATCCCGTTCGAACTGAGCAGCTATACC
AATGACAAGATTCTGATTCTGTATTTCAATAAACTGTATAAGAAGATCAAGGATAACAGCATTCTGGATATGCGT
TACGAAAACAATAAGTTTATCGACATTTCTGGTTACGGCAGCAACATTTCCATCAATGGCGATGTCTACATCTAC
AGCACCAATCGCAACCAGTTCGGCATCTACTCTAGCAAACCGAGCGAAGTTAACATCGCACAGAACAATGATATT
ATTTATAACGGTCGTTATCAAAACTTCTCTATCAGCTTTTGGGTCCGTATCCCGAAGTACTTCAATAAAGTCAAT
CTGAATAATGAATACACGATCATCGACTGCATTCGCAATAACAACAGCGGTTGGAAAATCAGCCTGAATTACAAC
AAAATTATTTGGACCCTGCAAGATACGGCGGGTAACAATCAGAAACTGGTGTTTAACTACACGCAAATGATCAGC
ATTTCTGACTATATCAACAAGTGGATCTTTGTTACCATCACCAATAATCGTCTGGGCAATAGCCGTATTTACATC
AACGGTAACCTGATTGATGAGAAAAGCATCAGCAACCTGGGCGATATTCACGTCAGCGACAACATTCTGTTCAAA
ATTGTTGGTTGTAACGATACCCGTTACGTCGGCATCCGTTATTTCAAGGTTTTCGATACGGAGCTGGGTAAAACG
GAAATCGAAACGTTGTACTCCGATGAACCAGATCCGAGCATTCTGAAGGACTTTTGGGGTAACTACTTGCTGTAC
AATAAACGTTACTATCTGCTGAATCTGTTGCGCACCGACAAGAGCATTACCCAAAACAGCAATTTCCTGAACATT
AATCAGCAACGCGGCGTATACCAAAAACCGAACATCTTCAGCAATACGCGCCTGTATACTGGTGTTGAAGTGATC
ATTCGTAAGAACGGTAGCACCGACATTAGCAACACGGACAATTTCGTCCGTAAGAATGACCTGGCGTACATTAAC
GTCGTGGACCGTGATGTCGAGTATCGTCTGTACGCAGACATCAGCATTGCGAAACCGGAAAAGATTATCAAGCTG
ATCCGTACCAGCAACAGCAACAACAGCCTGGGTCAGATCATTGTGATGGACAGCATTGGTAATAACTGCACGATG
AACTTCCAGAACAACAATGGTGGTAATATCGGTCTGCTGGGTTTTCACAGCAATAATCTGGTTGCTTCCAGCTGG
TACTACAATAACATTCGTAAAAACACGTCTAGCAATGGTTGTTTTTGGAGCTTTATCAGCAAAGAGCACGGCTGG
CAAGAAAAT
Polypeptide Sequence of rBoNT/F(0)
SEQ ID NO: 16
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTFNDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QEN
Nucleotide Sequence of rBoNT/A(0) (His-taqqed)
SEQ ID NO: 17
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAAAGCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACACCAGCTG
ATTTATGCCGGTCATCGTCTGTATGGTATTGCCATTAATCCGAATCGTGTGTTCAAAGTGAATACCAACGCCTAT
TATGAAATGAGCGGTCTGGAAGTGAGTTTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCTTTACCGAATACATTAAGAACATCATCAAC
ACCAGCATTCTGAACCTGCGTTATGAAAGCAATCATCTGATTGATCTGAGCCGTTATGCCAGCAAAATCAATATA
GGCAGCAAGGTTAACTTCGACCCGATTGACAAAAATCAGATACAGCTGTTTAATCTGGAAAGCAGCAAAATTGAG
GTGATCCTGAAAAACGCCATTGTGTATAATAGCATGTACGAGAATTTCTCGACCAGCTTTTGGATTCGTATCCCG
AAATACTTTAATAGCATCAGCCTGAACAACGAGTACACCATTATTAACTGCATGGAAAACAATAGCGGCTGGAAA
GTTAGCCTGAATTATGGCGAAATTATCTGGACCCTGCAGGATACCCAAGAAATCAAACAGCGTGTGGTTTTCAAA
TACAGCCAGATGATTAATATCAGCGACTATATCAACCGCTGGATTTTTGTGACCATTACCAATAATCGCCTGAAT
AACAGCAAGATCTATATTAACGGTCGTCTGATTGACCAGAAACCGATTAGTAATCTGGGTAATATTCATGCGAGC
AACAACATCATGTTTAAACTGGATGGTTGTCGTGATACCCATCGTTATATTTGGATCAAGTACTTCAACCTGTTC
GATAAAGAGTTGAACGAAAAAGAAATTAAAGACCTGTATGATAACCAGAGCAACAGCGGTATTCTGAAGGATTTT
TGGGGAGATTATCTGCAGTATGACAAACCGTATTATATGCTGAATCTGTACGACCCGAATAAATACGTGGATGTG
AATAATGTTGGCATCCGTGGTTATATGTACCTGAAAGGTCCGCGTGGTAGCGTTATGACCACAAACATTTATCTG
AATAGCAGCCTGTATCGCGGAACCAAATTCATCATTAAAAAGTATGCCAGCGGCAACAAGGATAATATTGTGCGT
AATAATGATCGCGTGTACATTAACGTTGTGGTGAAGAATAAAGAATATCGCCTGGCAACCAATGCAAGCCAGGCA
GGCGTTGAAAAAATTCTGAGTGCCCTGGAAATTCCGGATGTTGGTAATCTGAGCCAGGTTGTTGTGATGAAAAGC
AAAAATGATCAGGGCATCACCAACAAGTGCAAAATGAATCTGCAGGACAATAACGGCAACGATATTGGTTTTATT
GGCTTCCACCAGTTCAACAATATTGCGAAACTGGTTGCAAGCAATTGGTATAATCGTCAGATTGAACGTAGCAGT
CGTACCCTGGGTTGTAGCTGGGAATTTATCCCTGTGGATGATGGTTGGGGTGAACGTCCGCTGGAAAACCTGTAT
TTTCAAGGTGCAAGTCATCATCACCATCACCACCATCATTAA
Polypeptide Sequence of rBoNT/A(0) (His-taqqed)
SEQ ID NO: 18
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPLENLYFQGASHHHHHHHH
Nucleotide Sequence of rLH_{N}/A (His-taqqed)
SEQ ID NO: 19
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCGAAAATCTGTATTTCCAGGGTGCAAGTCAT
CATCACCATCACCACCATCATTAA
Polypeptide Sequence of rLH_{N}/A (His-taqqed)
SEQ ID NO: 20
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTENLYFQGASHHHHHHHH
Nucleotide Sequence of rH_{C}/A (His-taqqed)
SEQ ID NO: 21
ATGCATCATCACCATCACCACGAAAATCTATACTTCCAAGGAAAAAACATCATCAATACTAGCATTCTGAACCTG
CGTTACGAGAGCAATCATCTGATTGATCTGAGCCGTTATGCAAGCAAGATCAACATCGGTAGCAAGGTCAATTTT
GACCCGATCGATAAGAACCAGATCCAGCTGTTTAATCTGGAATCGAGCAAAATTGAGGTTATCCTGAAAAACGCC
ATTGTCTACAACTCCATGTACGAGAATTTCTCCACCAGCTTCTGGATTCGCATCCCGAAATACTTCAACAGCATT
AGCCTGAACAACGAGTATACTATCATCAACTGTATGGAGAACAACAGCGGTTGGAAGGTGTCTCTGAACTATGGT
GAGATCATTTGGACCTTGCAGGACACCCAAGAGATCAAGCAGCGCGTCGTGTTCAAGTACTCTCAAATGATCAAC
ATTTCCGATTACATTAATCGTTGGATCTTCGTGACCATTACGAATAACCGTCTGAATAACAGCAAGATTTACATC
AATGGTCGCTTGATCGATCAGAAACCGATTAGCAACCTGGGTAATATCCACGCAAGCAACAACATTATGTTCAAA
TTGGACGGTTGCCGCGATACCCATCGTTATATCTGGATCAAGTATTTCAACCTGTTTGATAAAGAACTGAATGAG
AAGGAGATCAAAGATTTGTATGACAACCAATCTAACAGCGGCATTTTGAAGGACTTCTGGGGCGATTATCTGCAA
TACGATAAGCCGTACTATATGCTGAACCTGTATGATCCGAACAAATATGTGGATGTCAATAATGTGGGTATTCGT
GGTTACATGTATTTGAAGGGTCCGCGTGGCAGCGTTATGACGACCAACATTTACCTGAACTCTAGCCTGTACCGT
GGTACGAAATTCATCATTAAGAAATATGCCAGCGGCAACAAAGATAACATTGTGCGTAATAACGATCGTGTCTAC
ATCAACGTGGTCGTGAAGAATAAAGAGTACCGTCTGGCGACCAACGCTTCGCAGGCGGGTGTTGAGAAAATTCTG
AGCGCGTTGGAGATCCCTGATGTCGGTAATCTGAGCCAAGTCGTGGTTATGAAGAGCAAGAACGACCAGGGTATC
ACTAACAAGTGCAAGATGAACCTGCAAGACAACAATGGTAACGACATCGGCTTTATTGGTTTCCACCAGTTCAAC
AATATTGCTAAACTGGTAGCGAGCAATTGGTACAATCGTCAGATTGAGCGCAGCAGCCGTACTTTGGGCTGTAGC
TGGGAGTTTATCCCGGTCGATGATGGTTGGGGCGAACGTCCGCTGTAA
Polypeptide Sequence of rH_{C}/A (His-taqqed)
SEQ ID NO: 22
MHHHHHHENLYFQGKNIINTSILNLRYESNHLIDLSRYASKINIGSKVNFDPIDKNQIQLFNLESSKIEVILKNA
IVYNSMYENFSTSFWIRIPKYFNSISLNNEYTIINCMENNSGWKVSLNYGEIIWTLQDTQEIKQRVVFKYSQMIN
ISDYINRWIFVTITNNRLNNSKIYINGRLIDQKPISNLGNIHASNNIMFKLDGCRDTHRYIWIKYFNLFDKELNE
KEIKDLYDNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSWEFIPVDDGWGERPL
Nucleotide Sequence of rLC/A (His-taqqed)
SEQ ID NO: 23
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTTGAAGAG
AATCTGTATTTCCAGGGTGCAAGTCATCATCACCATCACCACCATCATTAA
Polypeptide Sequence of rLC/A (His-taqqed)
SEQ ID NO: 24
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEENLYFQGASHHHHHHHH
Nucleotide Sequence of rBoNT/FA(0) (His-taqqed)
SEQ ID NO: 25
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CACCAGCTGATTTATGTTCTGCATGGTCTGTATGGCACCTATATCACCGAATACAAAATTACCCCGAATGTGGTT
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACCGAAAGCAGCATTGCCTATCTGATCAATATCATCAACAACGAAACGATCCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAACTGAAGTACAACTGCATTCTGAACATCAAATATGAGATGGATCGT
GACAAACTGGTTGATAGCAGCGGTTATCGTAGCCGTATCAATATTGGTACAGGCGTCAAATTTAGCGAGATCGAT
AAAAATCAAGTGCAGCTGAGCAATCTGGAATCCAGCAAAATTGAAGTCATTCTGAATAACGGCGTCATCTATAAC
AGCATGTATGAAAACTTTTCGACCAGCTTTTGGATTCGCATTCCGAAATACTTTCGCAACATCAATAACGAGTAC
AAGATCATCAGCTGTATGCAGAATAATAGCGGTTGGGAAGTGAGCCTGAATTTTAGCAATATGAACTCGAAAATC
ATCTGGACCCTGCAGGATACCGAAGGTATCAAAAAAACCGTTGTGTTTCAGTACACCCAGAACATTAACATTAGC
GACTATATCAACCGCTGGATCTTTGTGACCATTACAAATAATCGTCTGAGCAACAGCAAAATCTACATTAATGGT
CGCCTGATCAACGAAGAAAGCATTAGCGATCTGGGTAATATCCATGCCAGCAACAACATTATGTTTAAACTGGAT
GGTTGCCGTGATCCGCATCGTTATATCTGGATTAAATACTTTAACCTGTTTGACAAAGAGCTGAACAAGAAAGAA
ATTAAAGATCTGTACGACAACCAGAGCAATAGCGGTATTCTGAAAGATTTCTGGGGTGATTATCTGCAGTATGAC
AAACCGTATTATATGCTGAATCTGTATGACCCGAATAAGTATCTGGATGTGAATAATGTTGGCATCCGTGGCTAT
ATGTATCTGAAAGGTCCGCGTGGTCGTATTGTGACCACCAACATTTATCTGAATAGCACCCTGTATATGGGCACC
AAATTCATCATTAAGAAATATGCCAGCGGCAACAAAGATAACATTGTGCGTAATAATGATCGCGTGTATATTAAC
GTGGTGGTGAAGAATAAAGAATATCGCCTGGCAACCAATGCAAGCCAGGCAGGCGTTGAAAAAATTCTGAGCGCA
GTTGAAATCCCGGATGTTGGTAATCTGAGCCAGGTTGTTGTGATGAAAAGCGAAAATGATCAGGGCATTCGCAAC
AAGTGTAAAATGAATCTGCAAGACAATAACGGCAACGATATTGGCTTTATCGGCTTTCACCAGTTTAATAACATT
GCAAAACTGGTGGCCAGCAACTGGTATAACCGTCAGATTGGTAAAGCAAGCCGTACCTTTGGTTGTAGCTGGGAA
TTTATCCCGGTTGATGATGGTTGGGGTGAAAGCAGCCTGGAAAATCTGTATTTCCAGGGTGCCAGTCATCATCAC
CACCATCACCATCACTGA
Polypeptide Sequence of rBoNT/FA(0) (His-taqqed)
SEQ ID NO: 26
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HQLIYVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDFTTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGELKYNCILNIKYEMDRDKLVDSSGYRSRINIGTGVKFSEID
KNQVQLSNLESSKIEVILNNGVIYNSMYENFSTSFWIRIPKYFRNINNEYKIISCMQNNSGWEVSLNFSNMNSKI
IWTLQDTEGIKKTVVFQYTQNINISDYINRWIFVTITNNRLSNSKIYINGRLINEESISDLGNIHASNNIMFKLD
GCRDPHRYIWIKYFNLFDKELNKKEIKDLYDNQSNSGILKDFWGDYLQYDKPYYMLNLYDPNKYLDVNNVGIRGY
MYLKGPRGRIVTTNIYLNSTLYMGTKFIIKKYASGNKDNIVRNNDRVYINVVVKNKEYRLATNASQAGVEKILSA
VEIPDVGNLSQVVVMKSENDQGIRNKCKMNLQDNNGNDIGFIGFHQFNNIAKLVASNWYNRQIGKASRTFGCSWE
FIPVDDGWGESSLENLYFQGASHHHHHHHH
Nucleotide Sequence of rLH_{N}/FA (His-taqqed)
SEQ ID NO: 27
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACAGAAAGCAGCATTGCCTACCTGATCAATATCATCAACAACGAAACCATTCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAAGAGAACCTGTATTTCCAGGGTGCCAGTCATCATCACCACCATCAC
CATCACTGA
Polypeptide Sequence of rLH_{N}/FA (His-taqqed)
SEQ ID NO: 28
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDFTTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGEENLYFQGASHHHHHHHH
Nucleotide Sequence of rH_{C}/FA (His-taqqed)
SEQ ID NO: 29
ATGCTGAAGTATAACTGCATCCTGAACATCAAATATGAGATGGATCGTGATAAACTGGTTGATAGCAGCGGTTAT
CGTAGCCGTATCAATATTGGCACCGGTGTGAAATTTAGCGAGATCGATAAAAATCAGGTGCAGCTGAGCAATCTG
GAAAGCAGCAAAATTGAAGTGATTCTGAATAACGGCGTGATCTACAATAGCATGTATGAAAACTTTTCGACCAGC
TTCTGGATTCGCATTCCGAAATACTTTCGCAACATCAACAACGAGTACAAGATTATCAGCTGTATGCAGAATAAT
AGCGGTTGGGAAGTTAGCCTGAATTTCAGCAATATGAACAGCAAAATCATTTGGACCCTGCAGGATACCGAAGGT
ATCAAAAAAACCGTTGTGTTTCAGTACACCCAGAACATTAACATCAGCGATTACATTAACCGCTGGATCTTTGTG
ACCATTACCAATAATCGTCTGAGCAACAGCAAGATCTATATTAACGGTCGCCTGATTAACGAAGAGAGCATTAGC
GATCTGGGTAATATTCATGCCAGCAACAACATCATGTTTAAACTGGATGGTTGTCGTGATCCGCATCGTTATATT
TGGATCAAATACTTCAACCTGTTTGATAAAGAACTGAACAAAAAAGAAATCAAAGACCTGTATGATAACCAGAGC
AATAGCGGCATTCTGAAAGATTTTTGGGGTGATTATCTGCAGTATGACAAACCGTATTACATGCTGAATCTGTAC
GATCCGAACAAATATCTGGATGTGAATAATGTGGGTATCCGTGGCTATATGTATCTGAAAGGTCCGCGTGGTCGT
ATTGTTACCACCAACATTTATCTGAATAGCACCCTGTATATGGGCACCAAATTCATCATTAAAAAGTATGCCAGC
GGCAACAAAGATAACATTGTGCGTAATAATGATCGCGTGTATATCAATGTGGTGGTGAAGAATAAAGAATATCGT
CTGGCCACCAATGCAAGCCAGGCAGGCGTTGAAAAAATTCTGAGCGCAGTTGAAATTCCGGATGTTGGTAATCTG
AGCCAGGTTGTTGTTATGAAAAGCGAAAATGATCAGGGCATTCGCAACAAATGCAAAATGAATCTGCAGGACAAT
AACGGCAACGATATTGGTTTTATTGGCTTCCACCAGTTCAACAACATTGCAAAACTGGTGGCGAGCAATTGGTAT
AATCGTCAGATTGGTAAAGCAAGCCGTACCTTTGGTTGTAGCTGGGAATTTATTCCGGTTGATGATGGTTGGGGT
GAAAGCAGCCTGGAAAATCTGTATTTTCAGGGTGCAAGTCATCATCACCACCATCACCATCATTAA
Polypeptide Sequence of rH_{C}/FA (His-taqqed)
SEQ ID NO: 30
MLKYNCILNIKYEMDRDKLVDSSGYRSRINIGTGVKFSEIDKNQVQLSNLESSKIEVILNNGVIYNSMYENFSTS
FWIRIPKYFRNINNEYKIISCMQNNSGWEVSLNFSNMNSKIIWTLQDTEGIKKTVVFQYTQNINISDYINRWIFV
TITNNRLSNSKIYINGRLINEESISDLGNIHASNNIMFKLDGCRDPHRYIWIKYFNLFDKELNKKEIKDLYDNQS
NSGILKDFWGDYLQYDKPYYMLNLYDPNKYLDVNNVGIRGYMYLKGPRGRIVTTNIYLNSTLYMGTKFIIKKYAS
GNKDNIVRNNDRVYINVVVKNKEYRLATNASQAGVEKILSAVEIPDVGNLSQVVVMKSENDQGIRNKCKMNLQDN
NGNDIGFIGFHQFNNIAKLVASNWYNRQIGKASRTFGCSWEFIPVDDGWGESSLENLYFQGASHHHHHHHH
Nucleotide Sequence of rLC/FA (His-taqqed)
SEQ ID NO: 31
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCGAAAATCTGTATTTTCAGGGTGCCAGTCATCATCACCACCATCACCATCACTGA
Polypeptide Sequence of rLC/FA (His-taqqed)
SEQ ID NO: 32
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSENLYFQGASHHHHHHHH
Nucleotide Sequence of rBoNT/F(0) (His-taqqed)
SEQ ID NO: 33
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACACCAGCTGATTTATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAGATCAAGGACAACAGCATACTGGATATGCGC
TATGAAAACAACAAGTTCATTGATATCAGCGGCTATGGTAGCAACATTAGCATTAATGGTGATGTGTATATCTAC
AGCACCAACCGCAATCAGTTTGGTATTTATAGCAGCAAACCGAGCGAAGTTAATATTGCGCAGAATAACGATATC
ATCTACAACGGTCGCTATCAGAACTTTAGCATTAGCTTTTGGGTTCGCATTCCGAAATACTTTAACAAGGTGAAC
CTGAACAACGAGTACACCATTATTGATTGCATTCGCAATAATAACAGCGGCTGGAAAATCAGCCTGAACTATAAC
AAAATTATCTGGACCCTGCAGGATACCGCAGGTAATAATCAGAAACTGGTGTTTAACTACACCCAGATGATTAGC
ATCAGCGACTATATCAACAAATGGATCTTTGTGACCATTACCAACAATCGTCTGGGTAACAGCCGCATTTATATC
AATGGCAATCTGATCGACGAAAAAAGCATTTCAAATCTGGGCGATATTCACGTGAGCGATAACATTCTGTTCAAA
ATTGTTGGCTGCAACGATACCCGTTATGTTGGTATTCGTTACTTCAAAGTGTTTGATACGGAACTGGGCAAAACG
GAAATTGAAACCCTGTATAGTGATGAACCGGATCCGAGCATTCTGAAAGATTTTTGGGGTAATTATCTGCTGTAC
AACAAACGCTACTATCTGCTGAACCTGCTGCGTACCGATAAAAGCATTACACAGAATAGCAACTTTCTGAACATC
AATCAGCAGCGTGGTGTTTATCAGAAACCGAACATTTTTAGCAACACCCGTCTGTATACCGGTGTGGAAGTTATT
ATTCGTAAAAACGGTAGCACCGATATCAGCAACACCGATAACTTTGTGCGTAAAAATGACCTGGCCTATATTAAC
GTTGTTGATCGTGATGTTGAGTATCGTCTGTATGCGGATATTAGCATTGCCAAACCGGAAAAGATTATCAAACTG
ATCCGTACCAGCAACAGCAATAATTCACTGGGTCAGATTATCGTGATGGACAGCATTGGTAACAATTGCACCATG
AATTTCCAGAACAATAACGGTGGTAATATTGGCCTGCTGGGCTTTCATAGCAATAATCTGGTTGCAAGCAGCTGG
TATTACAACAACATCCGTAAAAATACCAGCAGTAATGGTTGCTTTTGGAGCTTTATCAGTAAAGAACATGGCTGG
CAAGAAAACGAGAACCTGTATTTTCAGGGTGCAAGTCATCATCACCATCACCACCATCATTAA
Polypeptide Sequence of rBoNT/F(0) (His-taqqed)
SEQ ID NO: 34
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTFNDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QENENLYFQGASHHHHHHHH
Nucleotide Sequence of rL_{H}N/F (His-taqqed)
SEQ ID NO: 35
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAAGAAAACCTGTATTTTCAGGGTGCAAGCCAT
CATCACCACCATCACCATCATTAA
Polypeptide Sequence of rL_{H}N/F (His-taqqed)
SEQ ID NO: 36
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTFNDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKENLYFQGASHHHHHHHH
Nucleotide Sequence of rH_{C}/F (His-taqqed)
SEQ ID NO: 37
ATGATCAAGGATAACAGCATTCTGGATATGCGCTATGAGAACAACAAATTCATTGATATTAGCGGCTATGGCAGC
AACATTAGCATTAATGGTGATGTGTATATCTACAGCACCAACCGTAATCAGTTTGGCATTTATAGCAGCAAACCG
AGCGAAGTTAATATTGCCCAGAACAACGATATCATCTATAACGGTCGCTATCAGAACTTCAGCATTAGCTTTTGG
GTTCGCATTCCGAAATACTTCAATAAGGTGAACCTGAACAACGAGTATACCATCATTGATTGCATTCGCAATAAT
AACAGCGGCTGGAAAATTAGCCTGAACTACAACAAAATTATCTGGACCCTGCAGGATACCGCAGGTAATAATCAG
AAACTGGTGTTTAACTACACCCAGATGATTAGCATCAGCGACTATATCAACAAATGGATCTTTGTGACCATTACC
AATAATCGCCTGGGTAATAGCCGCATTTATATCAATGGTAACCTGATCGATGAGAAAAGCATTAGCAATCTGGGT
GATATTCATGTGAGCGATAACATCCTGTTTAAAATCGTGGGTTGTAACGATACCCGTTATGTTGGTATTCGCTAC
TTCAAAGTGTTTGATACCGAACTGGGTAAAACCGAAATTGAAACCCTGTATAGTGATGAACCGGATCCGAGCATT
CTGAAAGATTTTTGGGGTAATTATCTGCTGTACAACAAACGCTACTATCTGCTGAATCTGCTGCGTACCGATAAA
TCAATTACCCAGAATAGCAACTTCCTGAACATTAATCAGCAGCGTGGTGTTTATCAGAAACCGAACATTTTTAGC
AACACCCGTCTGTATACCGGTGTGGAAGTTATTATTCGTAAAAATGGCAGCACCGATATCAGCAACACCGATAAC
TTTGTTCGCAAAAATGATCTGGCGTATATCAACGTTGTTGATCGTGATGTTGAATATCGTCTGTATGCCGATATT
AGCATTGCCAAACCGGAAAAAATCATCAAACTGATCCGTACCAGCAACAGCAATAATTCACTGGGTCAGATTATT
GTGATGGATAGCATTGGTAATAACTGCACCATGAACTTTCAGAACAATAACGGTGGTAATATTGGTCTGCTGGGC
TTTCATAGTAATAATCTGGTTGCAAGCAGCTGGTATTATAACAACATCCGTAAAAATACCAGCAGCAATGGTTGC
TTTTGGAGCTTTATTAGCAAAGAACATGGCTGGCAAGAAAACGAGAATCTGTATTTTCAGGGTGCAAGTCATCAT
CACCACCATCACCATCATTAA
Polypeptide Sequence of rH_{C}/F (His-taqqed)
SEQ ID NO: 38
MIKDNSILDMRYENNKFIDISGYGSNISINGDVYIYSTNRNQFGIYSSKPSEVNIAQNNDIIYNGRYQNFSISFW
VRIPKYFNKVNLNNEYTIIDCIRNNNSGWKISLNYNKIIWTLQDTAGNNQKLVFNYTQMISISDYINKWIFVTIT
NNRLGNSRIYINGNLIDEKSISNLGDIHVSDNILFKIVGCNDTRYVGIRYFKVFDTELGKTEIETLYSDEPDPSI
LKDFWGNYLLYNKRYYLLNLLRTDKSITQNSNFLNINQQRGVYQKPNIFSNTRLYTGVEVIIRKNGSTDISNTDN
FVRKNDLAYINVVDRDVEYRLYADISIAKPEKIIKLIRTSNSNNSLGQIIVMDSIGNNCTMNFQNNNGGNIGLLG
FHSNNLVASSWYYNNIRKNTSSNGCFWSFISKEHGWQENENLYFQGASHHHHHHHH
Nucleotide Sequence of rLC/F (His-taqqed)
SEQ ID NO: 39
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGAGAACCTGTATTTTCAGGGTGCAAGTCATCATCACCATCACCACCATCATTAA
Polypeptide Sequence of rLC/F (His-taqqed)
SEQ ID NO: 40
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTFNDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSENLYFQGASHHHHHHHH
Nucleotide Sequence of Cationic rH_{C}/A (His-taqqed)
SEQ ID NO: 41
ATGATCATCAACACCAGCATTCTGAACCTGCGTTATGAAAGCAAACATCTGATTGATCTGAGCCGTTATGCCAGC
AAAATCAATATAGGCAGCAAGGTTAACTTCGACCCGATTGACAAAAATCAGATACAGCTGTTTAATCTGGAAAGC
AGCAAAATTGAGGTGATCCTGAAAAAAGCGATCGTGTATAATAGCATGTACGAGAATTTTTCGACCAGCTTTTGG
ATTCGCATCCCGAAATACTTTAACAAGATTAGCCTGAACAACGAGTATACCATCATTAACTGCATGGAAAACAAT
AGCGGTTGGAAAGTCAGCCTGAATTATGGCGAAATTATCTGGACCCTGCAGGATACCAAAGAAATCAAACAGCGT
GTGGTGTTCAAATACAGCCAGATGATTAATATCAGCGACTATATCAACCGCTGGATTTTTGTGACCATTACCAAT
AATCGGCTGAACAAGAGCAAGATCTATATTAACGGTCGTCTGATTGACCAGAAACCGATTAGTAATCTGGGTAAT
ATTCATGCGAGCAACAAAATCATGTTTAAACTGGATGGTTGCCGTGATACCCATCGTTATATTTGGATCAAATAC
TTCAACCTGTTCGATAAAGAGTTGAACGAAAAAGAAATTAAAGACCTGTACGATAACCAGAGCAATAGCGGCATA
CTGAAAGATTTTTGGGGAGATTATCTGCAGTATGACAAACCGTATTATATGCTGAATCTGTACGACCCGAATAAA
TACGTGGATGTTAATAATGTGGGCATCCGTGGTTATATGTACCTGAAAGGTCCGCGTGGTAGCGTTATGACCACA
AACATTTATCTGAATAGCAGCCTGTATCGCGGAACCAAATTCATCATTAAAAAGTATGCCAGCGGCAACAAGGAT
AATATTGTGCGTAATAATGATCGCGTGTACATTAACGTTGTGGTGAAGAATAAAGAATATCGCCTGGCAACCAAT
GCAAGCCAGGCAGGCGTTGAAAAAATTCTGAGTGCCCTGGAAATTCCGGATGTTGGTAATCTGAGCCAGGTTGTT
GTGATGAAAAGCAAAAACGATAAAGGCATCACCAACAAATGCAAGATGAATCTGCAGGACAATAACGGCAATGAT
ATTGGCTTCATTGGCTTTCACCAGTTTAACAACATTGCAAAACTGGTTGCGAGCAATTGGTATAATCGTCAGATT
GAACGTAGCAGTCGTACCCTGGGTTGTAGCTGGGAATTTATCCCTGTGGATGATGGTTGGGGTGAACGTCCGCTG
AAGCTTGCGGCCGCACTCGAGCACCACCACCACCACCACTGA
Polypeptide Sequence of Cationic rH_{C}/A (His-taqqed)
SEQ ID NO: 42
MIINTSILNLRYESKHLIDLSRYASKINIGSKVNFDPIDKNQIQLFNLESSKIEVILKKAIVYNSMYENFSTSFW
IRIPKYFNKISLNNEYTIINCMENNSGWKVSLNYGEIIWTLQDTKEIKQRVVFKYSQMINISDYINRWIFVTITN
NRLNKSKIYINGRLIDQKPISNLGNIHASNKIMFKLDGCRDTHRYIWIKYFNLFDKELNEKEIKDLYDNQSNSGI
LKDFWGDYLQYDKPYYMLNLYDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDKGITNKCKMNLQDNNGND
IGFIGFHQFNNIAKLVASNWYNRQIERSSRTLGCSWEFIPVDDGWGERPLKLAAALEHHHHHH
Nucleotide Sequence of rH_{C}/AB (His-taqqed)
SEQ ID NO: 43
ATGATTCTGAACAATATTATCCTGAACCTGCGTTACAAAGACAACAATCTGATCGATCTGAGCGGCTATGGTGCA
AAAGTTGAAGTCTACGACGGTGTCGAACTGAACGATAAAAACCAGTTCAAACTGACCTCATCGGCTAACTCAAAA
ATTCGTGTGACGCAGAACCAAAACATCATCTTCAACTCGGTCTTTCTGGACTTCAGCGTGTCTTTCTGGATTCGC
ATCCCGAAATATAAAAATGATGGCATCCAGAACTACATCCATAACGAATACACCATCATCAACTGTATGAAAAAC
AACAGTGGTTGGAAAATTTCCATCCGTGGCAACCGCATTATCTGGACCCTGATTGATATCAATGGTAAAACGAAA
AGCGTGTTTTTCGAATACAACATCCGTGAAGATATCTCTGAATACATCAATCGCTGGTTTTTCGTGACCATTACG
AACAATCTGAACAATGCGAAAATCTATATCAACGGCAAACTGGAAAGTAATACCGACATCAAAGATATTCGTGAA
GTTATCGCCAACGGTGAAATCATCTTCAAACTGGATGGCGACATCGATCGCACCCAGTTCATTTGGATGAAATAC
TTCTCCATCTTCAACACGGAACTGAGTCAGTCCAATATCGAAGAACGCTACAAAATCCAATCATACTCGGAATAC
CTGAAAGATTTCTGGGGTAACCCGCTGATGTACAACAAAGAATACTACATGTTCAACGCGGGCAACAAAAACTCA
TACATCAAACTGAAAAAAGATTCGCCGGTGGGTGAAATCCTGACCCGTAGCAAATACAACCAGAACTCTAAATAC
ATCAACTATCGCGATCTGTACATTGGCGAAAAATTTATTATCCGTCGCAAAAGCAACTCTCAGAGTATTAATGAT
GACATCGTGCGTAAAGAAGACTACATCTATCTGGATTTCTTTAATCTGAACCAAGAATGGCGCGTTTATACCTAC
AAATACTTCAAAAAAGAAGAAATGAAACTGTTCCTGGCCCCGATTTACGACAGCGATGAATTTTACAACACCATC
CAGATCAAAGAATACGATGAACAGCCGACGTATAGTTGCCAACTGCTGTTCAAAAAAGACGAAGAATCCACCGAT
GAAATTGGCCTGATTGGTATCCACCGTTTCTATGAAAGCGGTATCGTTTTCGAAGAATACAAAGATTACTTCTGT
ATCTCTAAATGGTATCTGAAAGAAGTCAAACGCAAACCGTACAACCTGAAACTGGGCTGCAACTGGCAATTTATC
CCGAAAGACGAAGGCTGGACCGAAAAGCTTGCGGCCGCACTCGAGCACCACCACCACCACCACTGA
Polypeptide Sequence of rH_{C}/AB (His-taqqed)
SEQ ID NO: 44
MILNNIILNLRYKDNNLIDLSGYGAKVEVYDGVELNDKNQFKLTSSANSKIRVTQNQNIIFNSVFLDFSVSFWIR
IPKYKNDGIQNYIHNEYTIINCMKNNSGWKISIRGNRIIWTLIDINGKTKSVFFEYNIREDISEYINRWFFVTIT
NNLNNAKIYINGKLESNTDIKDIREVIANGEIIFKLDGDIDRTQFIWMKYFSIFNTELSQSNIEERYKIQSYSEY
LKDFWGNPLMYNKEYYMFNAGNKNSYIKLKKDSPVGEILTRSKYNQNSKYINYRDLYIGEKFIIRRKSNSQSIND
DIVRKEDYIYLDFFNLNQEWRVYTYKYFKKEEMKLFLAPIYDSDEFYNTIQIKEYDEQPTYSCQLLFKKDEESTD
EIGLIGIHRFYESGIVFEEYKDYFCISKWYLKEVKRKPYNLKLGCNWQFIPKDEGWTEKLAAALEHHHHHH
Nucleotide Sequence of rH_{C}/A Variant Y1117V H1253K (His-taqqed)
SEQ ID NO: 45
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTTCaAaCAGTTCAACAATATTGCTAAACTGGTAGCGAGCAATTGGTACAATCGTCAGATT
GAGCGCAGCAGCCGTACTTTGGGCTGTAGCTGGGAGTTTATCCCGGTCGATGATGGTTGGGGCGAACGTCCGCTG
CACCATCACCATCACCATCACCATCACCATT
Polypeptide Sequence of rH_{C}/A Variant Y1117V H1253K (His-taqqed)
SEQ ID NO: 46
MIINTSILNLRYESNHLIDLSRYASKINIGSKVNFDPIDKNQIQLFNLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGFKQFNNIAKLVASNWYNRQIERSSRTLGCSWEFIPVDDGWGERPLHHHHHHHHHH
Nucleotide Sequence of rH_{C}/A Variant Y1117V F1252Y H1253K L1278F
(His-taqqed)
SEQ ID NO: 47
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTTTtGGCTGTAGCTGGGAGTTTATCCCGGTCGATGATGGTTGGGGCGAACGTCCGCTG
CACCATCACCATCACCATCACCATCACCATTAA
Polypeptide Sequence of rH_{C}/A Variant Y1117V F1252Y H1253K L1278F
(His-taqqed)
SEQ ID NO: 48
MIINTSILNLRYESNHLIDLSRYASKINIGSKVNFDPIDKNQIQLFNLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTFGCSWEFIPVDDGWGERPLHHHHHHHHHH
Nucleotide Sequence of rH_{C}/A Variant Y1117V F1252Y H1253K L1278H
(His-taqqed)
SEQ ID NO: 49
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTcatGGCTGTAGCTGGGAGTTTATCCCGGTCGATGATGGTTGGGGCGAACGTCCGCTG
CACCATCACCATCACCAT
Polypeptide Sequence of rH_{C}/A Variant Y1117V F1252Y H1253K L1278H
(His-taqqed)
SEQ ID NO: 50
MIINTSILNLRYESNHLIDLSRYASKINIGSKVNFDPIDKNQIQLFNLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTHGCSWEFIPVDDGWGERPLHHHHHH
Polypeptide Sequence of BoNT/A - UniProt P10845
SEQ ID NO: 51
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLFDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVWKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
Polypeptide Sequence of BoNT/B - UniProt P10844
SEQ ID NO: 52
MPVTINNFNYNDPIDNNNIIMMEPPFARGTGRYYKAFKITDRIWIIPERYTFGYKPEDFN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVFNNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGETETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
Polypeptide Sequence of BoNT/C - UniProt P18640
SEQ ID NO: 53
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVFNTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYR
HNYLVPTVKQGNYASLLESTSTHWGFVPVSE
Polypeptide Sequence of BoNT/D - UniProt P19321
SEQ ID NO: 54
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
VVYSSQYNVKNRTHYFSRHYLPVFANILDDNIYTIRDGFNLTNKGFNIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNENQAFATAGVAFLLEGEP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
Polypeptide Sequence of BoNT/E - UniProt Q00496
SEQ ID NO: 55
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRFNDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKFNDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNFNSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
Polypeptide Sequence of BoNT/F - UniProt A7GBG3
SEQ ID NO: 56
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
Polypeptide Sequence of BoNT/G - UniProt Q60393
SEQ ID NO: 57
MPVNIKXFNYNDPINNDDIIMMEPFNDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAAWANVSINKKIIQPGAEDQIKGLMTNLIIFGPGPVLSDNFTDSMIMNGH
SPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGFNIASKNLKTEFNGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDFNDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVFNNYISNISSNAILSLSYRGGRLIDSSGYGATMNVGSDVIFND
IGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMQNIYIKYFSKASMGETAPRTNFNNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
Polypeptide Sequence of TeNT - UniProt P04958
SEQ ID NO: 58
MPITINNFRYSDPVNNDTIIMMEPPYCKGLDIYYKAFKITDRIWIVPERYEFGTKPEDFN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMFNNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
Polypeptide Sequence of BoNT/X
SEQ ID NO: 59
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
Nucleotide Sequence of mrBoNT/A
SEQ ID NO: 60
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAAGCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
cGTACTTTGGGCTGTAGCTGGGAGTTTATCCCGGTCGATGATGGTTGGGGCGAACGTCCGCTG
Polypeptide Sequence of mrBoNT/A
SEQ ID NO: 61
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
Polypeptide Sequence of Unmodified BoNT/A1
SEQ ID NO: 62
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASOA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
Polypeptide Sequence of mrBoNT/AB
SEQ ID NO: 63
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
Polypeptide Sequence of mrBoNT/AB(0)
SEQ ID NO: 64
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
Polypeptide Sequence of mrBoNT/A(0)
SEQ ID NO: 65
MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHQLIYAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

### EXAMPLES

### Example 1

### Multiple Catalytically Inactive BoNT Serotypes Increase Total Neurite Length Compared to Untreated Control Cells

### Materials & Methods

Five catalytically inactive (i.e. endopeptidase inactive) botulinum neurotoxin (BoNT) serotypes were recombinantly expressed in E. coli, namely corresponding to serotypes A, B, C, E and F, and denoted as rBoNT/A(0), rBoNT/B(0), rBoNT/C(0), rBoNT/E(0), and rBoNT/F(0). As a result of being catalytically inactive, these molecules were not able to cleave their respective (SNARE) protein substrates.

A motor neuron-like hybrid cell line (NSC34 cells) (Tebu-Bio, Cedarlane laboratories, France) was cultured on poly-D-lysine coated black multiwells at 5000 cell/well and cultured in DMEM with added 10% FCS and penicillin/streptomycin. After plating, cells were differentiated into motor neurons by exposure to 1 uM retinoic acid and low serum for 4 days, then cells were treated with rBoNT/A(0), rBoNT/B(0), rBoNT/C(0), rBoNT/E(0) and rBoNT/F(0) at 3 different concentrations: 0.1, 1 and 10 nM for 4 days and fixed with paraformaldehyde 4%-sucrose 4%. Brain-derived neurotrophic factor (BDNF) (commercially available from ReproTech EC Ltd, London, UK) 1 ng/mL was used as a positive control of neuronal outgrowth. Cells were fixed with paraformaldehyde 4%-sucrose 4%, then stained with appropriate antibodies. In particular, Anti-8111 Tubulin mAb (Promega G7121) was diluted (1:1000) in 1×PBS+2% BSA+0.3% TritonX-100 and plates were incubated at 37° C. for 3 hours. Alexa Fluor 488 Goat anti-Mouse IgG (H+L) Secondary Antibody (Life Tech cat. A-11001) was then administered (1:2000 in 1×PBS+2% BSA+0.3% TritonX-100) for 1 h at 37° C. Nuclei were stained with DAPI. Image analysis: 6 images per well were taken with ArrayScan XTI HCA Reader (Thermo Fisher Scientific) with a 10× objective. All analysis was performed using Image J software (open source software from NIH, Maryland, USA). Three, independent experiments were carried out. Each independent experiment contained 6 replicates.

### Results

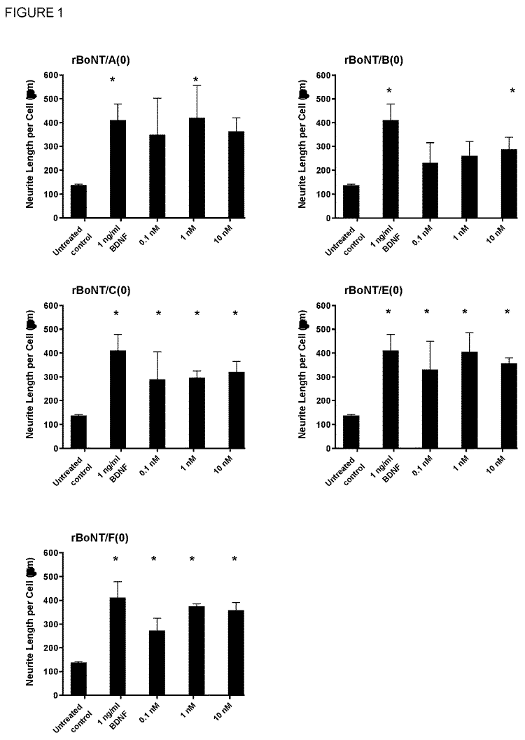

Cells were exposed to the different catalytically inactive BoNT serotypes for 4 days (FIG. 1). FIG. 1 shows the mean neurite outgrowth of NSC34 cells exposed to the three different concentrations. The graph presents the mean of the three independent experimental rounds. Data on mean neurite outgrowth confirms that rBoNT/A(0) increases neurite length per NSC34 cell when compared to an untreated control, similarly to positive control BDNF. rBoNT/B(0), rBoNT/C(0), rBoNT/E(0), and rBoNT/F(0) were also found to increase neurite length per NSC34 cell.

Thus, these data confirm that the neurotrophic properties of BoNT/A can also be extrapolated to other BoNT serotypes.

### Example 2

### BoNT L-Chain and LH_{N} Increase Total Neurite Length Vs. A Control

### Materials & Methods

Catalytically inactive botulinum toxin rBoNT/A(0) was recombinantly expressed in E. coli. Fragments of BoNT/A were also expressed in E. coli, and are denoted as light chain (L/A), light-chain and translocation domain (LH_{N}/A), and the cell binding domain fragment (H_{C}/A) of the heavy chain. NSC34 cells were exposed to the BoNT/A fragments as well as full-length rBoNT/A(0) as for Example 1.

### Results

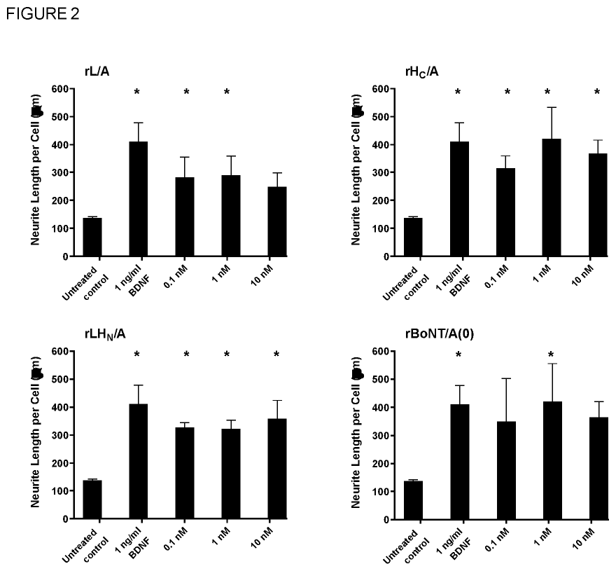

FIG. 2 shows the mean neurite outgrowth of NSC34 cells exposed to the three different concentrations of rBoNT/A(0), rL/A, rLH_{N}/A and rH_{C}/A. The graph presents the mean of the three independent experimental rounds.

Similarly to rH_{C}/A, both rL/A and rLH_{N}/A were found to increase neurite length per NSC34 cell at every concentration when compared to an untreated control, similarly to positive control BDNF. It was particularly unexpected that the rL/A and rLH_{N}/A fragments were neurotrophic, since both lack the clostridial toxin receptor binding domain (present in rH_{C}/A).

### Example 3

### Other Protein(s) Administered at a Similar Concentration to BoNT/a(0) or Fragments Thereof Did not Increase Neurite Outgrowth

### Materials & Methods

NSC34 cells were differentiated, then cultured for 4 days under the following experimental conditions: (1) Untreated cells control: cells underwent the same number of manipulations i.e. washes/feeding as compound treated cells however untreated control cells to be exposed to growth medium only, (2) BDNF— positive assay control, 1 ng/ml, (3) BoNT/A(0) at 3 doses (0.1, 1 and 10 nM), (4) Negative assay controls (protein controls): 1. A7030, Sigma, Bovine Serum Albumin (BSA), 2. NBP1-37082, Bio-techne, Recombinant Human Annexin A4 Protein, 3. U-100AT, Bio-techne, Recombinant Plant Ubiquitin Protein, 4. E. coli expression lysate, which does not contain botulinum neurotoxins or fragments thereof. All negative control proteins were tested at 1.5 ug/ml final concentration. This concentration corresponds to 10 nM of BoNT/A(0). Protein solutions were in PBS, except annexin 4-20 mM Tris-HCl buffer (pH8.0) containing 20% glycerol, 0.2M NaCl. All protein solutions were at 1 mg/ml. Cells were stained with Anti-Beta III Tubulin diluter 1:1000 in 1×PBS-4% BSA-0.3% TritonX100 and secondary antibody anti-mouse Alexa Fluor 488; DAPI was used as nuclear stain. All original images of beta 3-tubulin signal were processed using NeurphologyJ (an Image J macro, NIH, Maryland, USA).

### Results

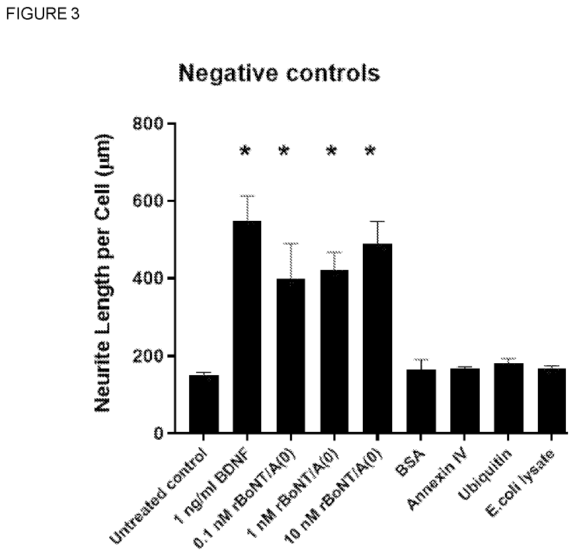

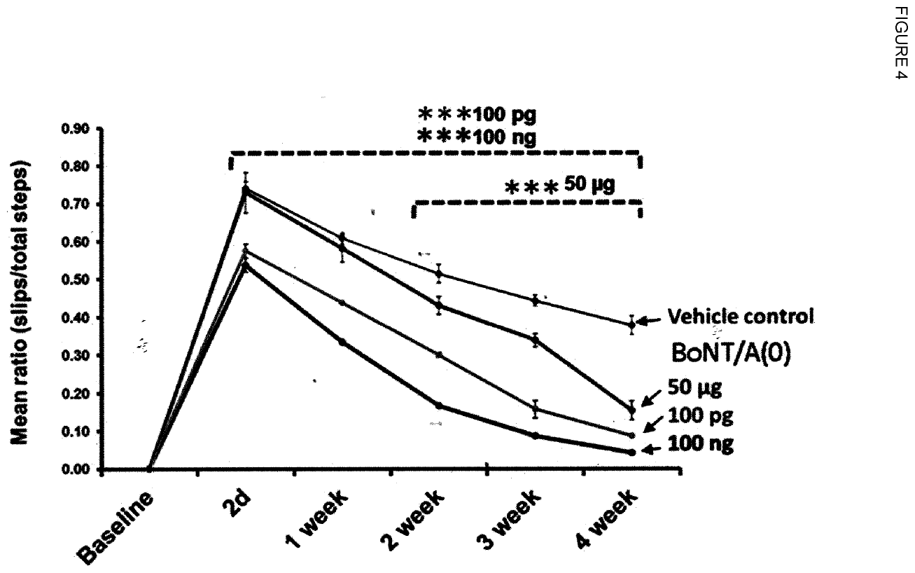

Cells were exposed to the different experimental conditions. FIG. 3 shows the mean neurite length in NSC34 cells. The graph presents the mean of the three independent experimental rounds. Data on mean neurite outgrowth confirm that while rBoNT/A(0) increases neurite length per NSC34 cell when compared to an untreated control, similarly to positive control BDNF. In contrast, none of the other ‘negative control’ conditions increased neurite length. This validates the neurotrophic effects observed upon exposure to rL/A and rLH_{N}/A (as well as the various BoNT serotypes and rH_{C}/A), and demonstrates that the effects do not simply arise from exposure of NSC34 cells to proteins or to putative residual E. coli components present in the botulinum toxin preparations.

### Example 4

### Treatment of a Neuronal Injury In Vivo

A study was designed to investigate the efficacy of catalytically inactive botulinum toxin rBoNT/A(0) in enhancing functional restoration and neuroregeneration using an in vivo mouse dorsal column lesion model. The model is useful for analysing the efficacy of molecules that cause local sprouting and/or long tract axon regeneration. As is well established, crushing injuries are a frequent scenario in spinal cord injury and therefore the model mimics most of the pathological changes that occur in the spinal cord after trauma (see Lagord et al, 2002; Molecular and Cellular Neuroscience 20:69; Esmaelli et al., 2014; Neural Regeneration Research 9:1653; Surey et al., 2014; Neuroscience 275C:62; Almutiri et al., 2018; Scientific Reports 8:10707 for details of the model and the injury responses).

### Materials & Methods

### Mouse Model of Spinal Cord Injury

Before surgery, C57/BL mice were injected subcutaneously with Buprenorphine and anaesthetised using 5% of Isoflurane in 1.8 m1/I of 02 with body temperature and heart rate monitored throughout surgery. After partial laminectomy at thoracic level 8 (T8) the ascending sensory, descending motor and segmental proprioceptive axons (SPA) of the spinal dorsal column (SDC) were crushed bilaterally using calibrated watchmakers' forceps 1 mm deep×1 mm wide.

### Drug Administration

rBoNT/A(0) administration was by way of a single intrathecal 10 μl injection (into the CSF of the spinal canal) of one of 3 doses (100 pg, 100 ng and 50 μg/mouse) at the time of surgery. Treatment groups for each of the 3 doses were as follows:

1. Vehicle (phosphate buffered saline [PBS]), i.e. SDC lesion plus an immediate single 10 μl intrathecal injection of vehicle; n=6 mice.

2. BoNT treated, i.e. SDC lesion plus an immediate single 10 μl intrathecal injection of one of 3 doses of BoNT (100 pg, 100 ng and 50 μg/mouse); 3×n=6/group; 18 mice.

Intrathecal injection of BoNT was carried out as follows. Mice were placed in the prone position and an injection made between L5 and 51 spinal vertebrae. The spinous processes were incised and reflected rostrally to reveal the ligamentum flavum and a blunt 25 G needle was inserted through the ligamentum flavum at an angle of 60° horizontal and access to the intrathecal space was confirmed by reflux of cerebrospinal fluid (CSF) and the presence of a ‘tail flick’. Then 10 μl of injectate was slowly injected over 1 min and CSF expression was facilitated by gentle tail elevation.

### Measured End-Points

1. Locomotor function was measured using the horizontal ladder walking test at baseline (prior to injury) then again at 2 d, 1 w, 2 w, 3 w and 4 w after SDC injury.

2. Qualitative histological assessment at the 4 w time-point of sprouting and regeneration from motor and sensory neurons/axons, i.e. axonal growth over short (<1 mm) and long (˜5 mm) distances. Tissue sections stained for Neurofilament 200 (NF200) detects mature axons. Phosphorylated MAP1b is present in growing axons and growth cones where it maintains a dynamic balance between cytoskeletal components and regulates the stability and interaction of microtubules and actin to promote axonal growth, neural connectivity and regeneration in the central nervous system. MAP1b staining reveals areas of active axonal sprouting.

### Horizontal Ladder Test

This tests locomotor function and is performed on a 0.6 metre long horizontal ladder with a width of 8 cm and randomly adjusted rungs with variable gaps of 1-2 cm. Prior to injury, then again at 2 d, 1 w, 2 w, 3 w and 4 w after SDC injury, mice were assessed traversing the ladder and the left and right rear paw slips were recorded along with the total number of steps by an individual unaware of the treatment group. To calculate the mean error rate, the number of slips was divided by the total number of steps.

### Tissue Preparation and Cryo-Sectioning

At 4 w after SDC lesion, mice were intracardially perfused with 4% formaldehyde (Raymond A Lamb, Peterborough, UK) and dissected segments of T8 cord containing the DC injury sites (lesion site+5 mm either side) together with the Tibialis Cranialis muscles were post-fixed for 2 h at RT, cryoprotected in a graded series of sucrose, blocked up in optimal cutting temperature medium (OCT; Raymond A Lamb) and sectioned at 15 μm thick using a Bright cryostat.

### Immunohistochemistry

Sections were thawed at room temperature for 30 min before washing twice in 0.1M phosphate buffered saline, pH7.4 (PBS; Raymond A Lamb). Sections were then permeablised in 0.1% Triton X-100 in PBS (Sigma) for 10 min and blocked in PBS containing 0.5% bovine serum albumin (BSA) and 0.1% Triton-X100 (all from Sigma) for 30 min at room temperature. Sections were then incubated with the appropriate primary antibody diluted with antibody diluting buffer (ADB; PBS containing 0.5% BSA and 0.05% Tween-20 (all from Sigma)) and incubated overnight at 4° C. in a humidified chamber. Sections were then washed in PBS and incubated with appropriate fluorescently-labelled secondary antibody diluted in ADB. Sections were then washed in PBS and coverslips mounted using Vectashield containing DAPI (Vector Laboratories, Peterborough, UK). Negative controls were included in each run that included omission of primary antibody and these were used to set the background threshold levels for image capture. Sections were viewed and images captured using an Axioplan 2 epifluorescent microscope equipped with an Axiocam HRc running Axiovision software.

Primary antibodies used were as follows:
Rabbit anti-NF200 Sigma, Poole, UK (1:300 dilution)
Rabbit MAP1b Abcam, Cambridge, UK (1:400 dilution)

Secondary antibodies used were as follows:
Alexa 488 anti-rabbit IgG Invitrogen, Paisley, UK (1:400 dilution)
Alexa 594 anti rabbit IgG Invitrogen, Paisley, UK (1:400 dilution)

### Statistics

Statistical analyses on the functional data were performed using SPSS 20 (IBM, USA). Normal distribution tests were carried out to determine the most appropriate statistical analysis to compare treatments. Statistical significance was determined at p<0.05.

### Results

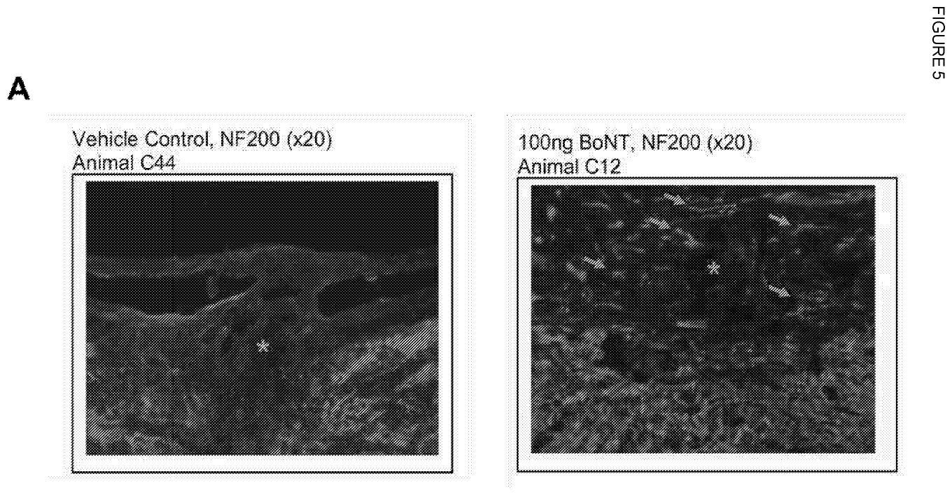

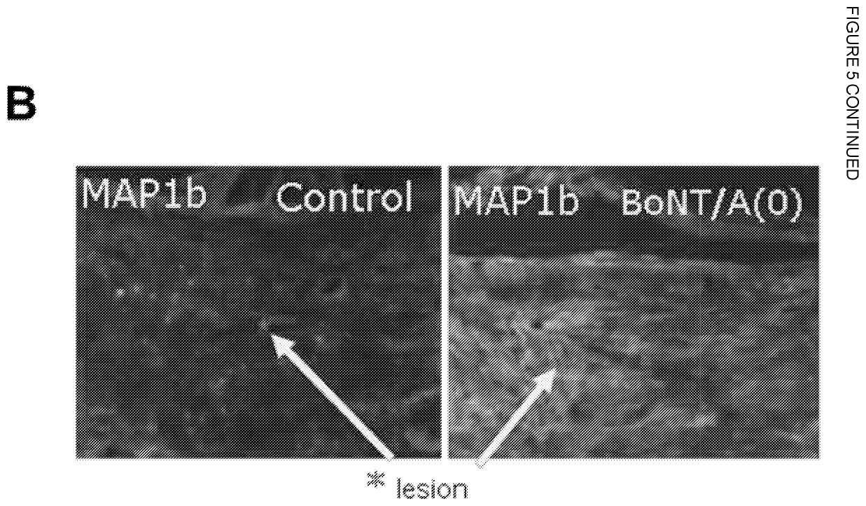

FIG. 4 shows that administration of rBoNT/A(0) reduced the extent of dorsal-column injury induced locomotor deficits at day 2 when compared to vehicle control for the 100 pg and 100 ng doses. Administration of rBoNT/A(0) significantly reduced dorsal column injury-induced locomotor deficits at 4 weeks and the rate of recovery when compared to vehicle control at all dosages tested. Furthermore, the effects were more pronounced when rBoNT/A(0) was administered intrathecally than when administered intraspinally (data not shown).

The immunohistochemical assessment employed the use of antibodies to Neurofilament 200 (NF200) and MAP1b. Neurofilament 200 (NF200) is expressed in mature axons and the pMAP1b antibody reveals neurofilaments in the terminals of actively sprouting axons, illustrating axons that are still actively sprouting around and within the lesion site.

FIG. 5A shows that many NF200 stained axons were visible surrounding the lesion site of vehicle-treated animals, with few if any NF200+ axons present within the core of the lesion site in untreated animals. By contrast, many NF200 stained axons were visible surrounding the lesion site of rBoNT/A(0)-treated animals, with numerous NF200+ axons also visible within the core of the lesion site.

FIG. 5B shows that modest numbers of MAP1b stained sprouting axons were visible surrounding the lesion site of vehicle-treated animals, with little if any MAP1b axons present within the core of the lesion site. In contrast, MAP1b staining revealed florid axonal sprouting around the lesion site and also ramifying throughout the core of the lesion site in the rBoNT/A(0)-treated animals.

The rapidity of the onset of improvement in performance in the functional test shows that rBoNT/A(0) caused axonal sprouting with the establishment of useful functional synapses below the lesion. Qualitative immunohistochemistry provided evidence of BoNT-induced florid axonal sprouting locally through the SDC lesion site.

These in vivo data are clear evidence validating a role for rBoNT/A(0) in the treatment of neurological disorders.

### Example 5

### The Effect of Full-Length Catalytically-Inactive Recombinant BoNTs, BoNT Fragments, & Variants on Neurite Number per Cell

A number of full-length catalytically-inactive recombinant BoNT serotypes, as well as BoNT fragments, and variants were tested for their modulatory action on neurite outgrowth in vitro.

### Materials & Methods

Cells exposed to the polypeptides were compared to those exposed to a positive control (1 ng/ml BDNF). Mouse Motor Neuron-Like Hybrid (NSC34) cells were differentiated and exposed during 4 days in vitro (DIV) to different polypeptides at 3 different doses (0.1 nM, 1 nM, and 10 nM).

NSC34 cells were produced by fusion of motor neuron enriched, embryonic mouse spinal cord cells and mouse neuroblastoma (Cashman et al. Dev Dyn. 1992 July; 194(3):209-21, which is incorporated herein by reference). Said cells mimic many properties of motor neurons, including choline acetyltransferase, acetylcholine synthesis, storage and release and neurofilament triplet proteins. Moreover, NSC34 spinal cord motor neurons express glutamate receptor proteins and generate action potentials. NSC34 neurons have been widely used to study mechanisms of neuron signalling and neuron degeneration.

The following experimental scheme was adopted: Screening on Neuronal cell line (NSC34):

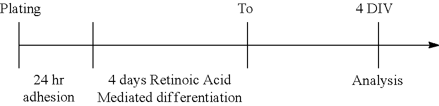

NSC34 cells were cultivated on poly-D-lysine-coated glass coverslips in DMEM plus 10% FCS.

After plating, cells were differentiated into motor neurons by exposure to retinoic acid and low serum levels for 4 days. Cells were cultured either in the presence/absence of the polypeptides at a specific timepoint. (i.e. 4 DIV). Test data was compared with effects seen on positive (BDNF) and also negative (BSA) control data.

After 4 days in vitro (DIV), cells were fixed in 4% paraformaldehyde, stained with specific neuronal markers (beta tubulin) and quantitatively assayed for neurite outgrowths (neurite extension, axonal elongation, arborization). Image acquisition was carried out using Operetta CLS HCS microscope (PerkinElmer) by means of a 20× objective. Per each well, six (6) fields-of-view were acquired. The neurite outgrowth analysis was performed and the mean neurites per cell assessed.

### Results

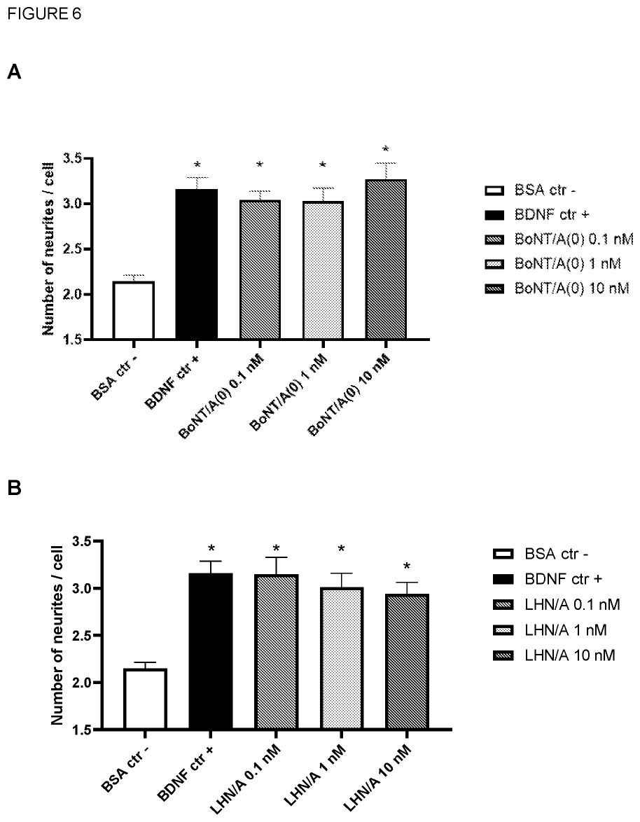

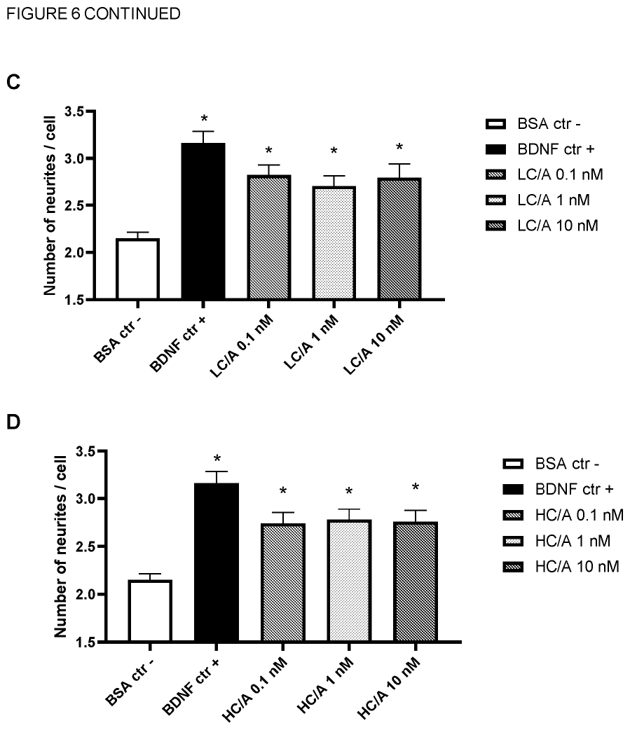

FIGS. 6-10 represent the mean value of the number of neurites counted on each cell, evaluated in three independent experimental sessions. Data were normalized on untreated control cells. The polypeptides statistically-significantly increased the number of neurites per cell when compared to BSA.

For BoNT/A, the LH_{N}/A fragment (light-chain plus translocation domain) had improved activity compared to the cell binding domain (H_{C} domain) fragment (see FIG. 6).

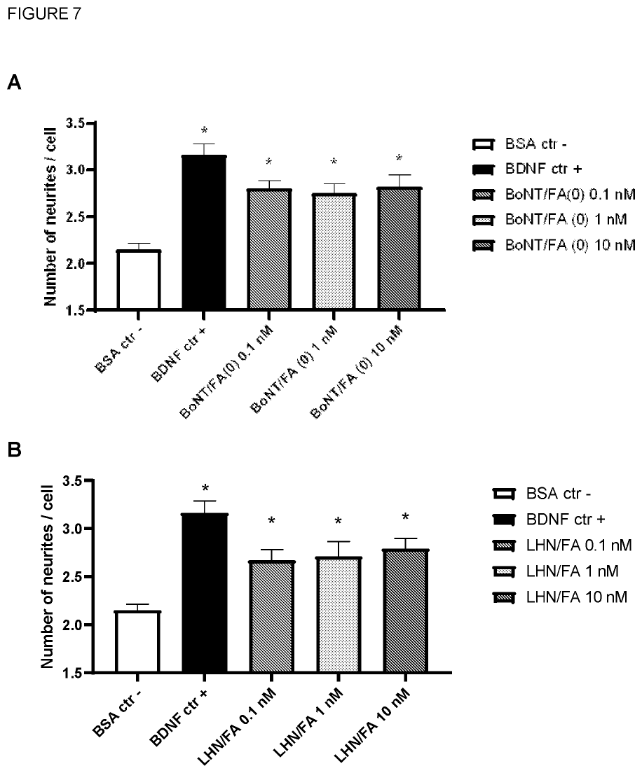

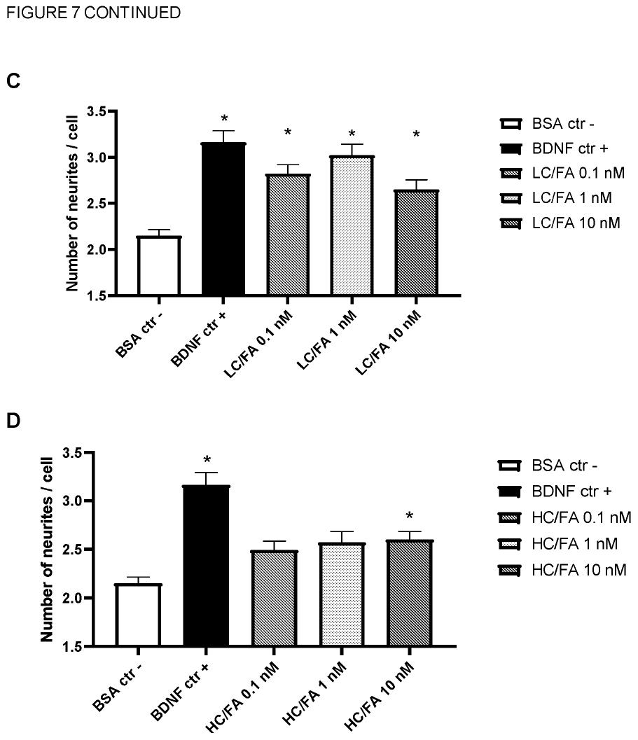

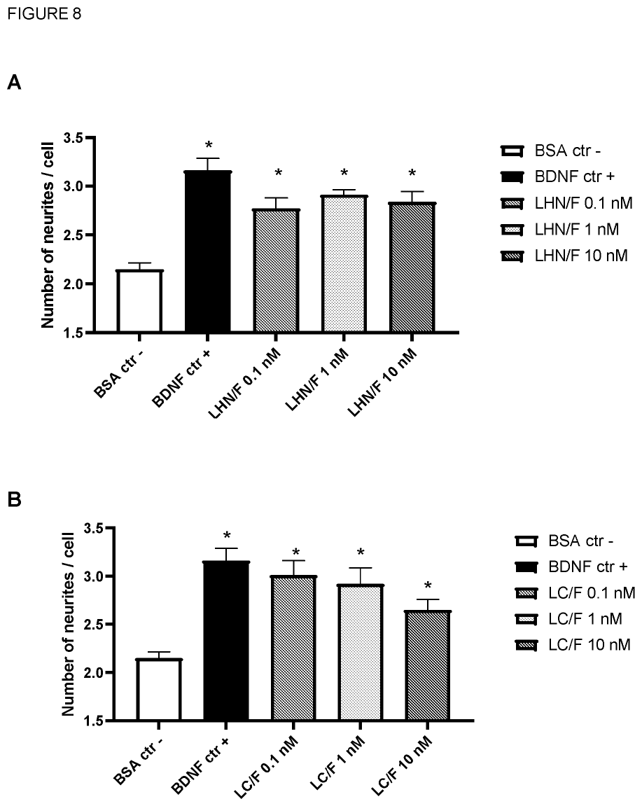

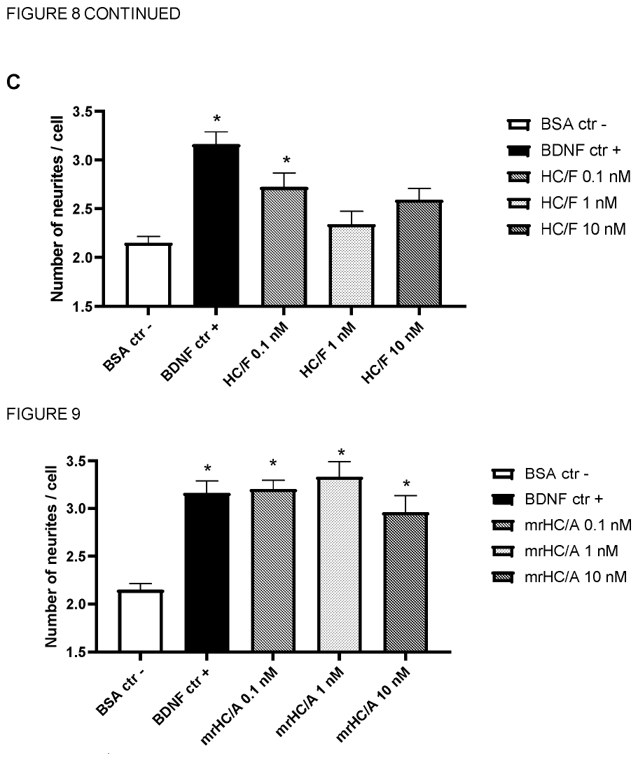

For both BoNT/FA and BoNT/F, the LH_{N} and LC (light-chain only) fragments showed improved activity compared to the H_{C} domain fragments (see FIGS. 7 and 8).

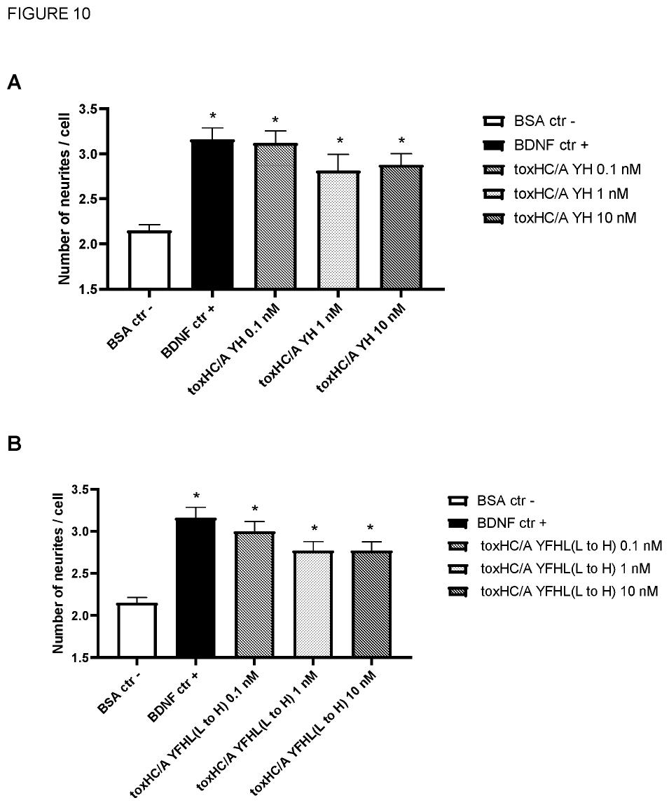

Finally, the variant H_{C} domain fragments were all shown to be highly efficacious (FIGS. 9 and 10), with the cationic H_{C}/A domain (SEQ ID NO: 42—FIG. 9) exhibiting exceptional activity, which at 2 of 3 concentrations was improved versus BDNF. It is expected that the high activity of the cationic H_{C}/A domain would also be evident in full-length polypeptides comprising said domain (whether catalytically inactive or active).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### Clauses

1. A polypeptide for use in promoting neuronal growth or neuronal repair to treat a neurological disorder in a subject, wherein the polypeptide comprises:
   a clostridial neurotoxin light chain (L-chain) or fragment thereof; and/or
   a fragment of a clostridial neurotoxin heavy chain (H-chain).
2. A method for promoting neuronal growth or neuronal repair to treat a neurological disorder in a subject, the method comprising administering a polypeptide to the subject, wherein the polypeptide comprises:
   a clostridial neurotoxin L-chain or fragment thereof; and/or
   a fragment of a clostridial neurotoxin H-chain.
3. Use of a polypeptide in the manufacture of a medicament for promoting neuronal growth or neuronal repair to treat a neurological disorder in a subject, wherein the polypeptide comprises:
   a clostridial neurotoxin L-chain or fragment thereof; and/or
   a fragment of a clostridial neurotoxin H-chain.
4. The polypeptide for use according to clause 1, method according to clause 2 or use according to clause 3, wherein the L-chain is catalytically inactive.
5. The polypeptide for use, method or use according to any one of the preceding clauses, wherein the polypeptide consists essentially of a clostridial neurotoxin light chain (L-chain) or fragment thereof; and/or a fragment of a clostridial neurotoxin heavy chain (H-chain).
6. The polypeptide for use, method or use according to any one of the preceding clauses, wherein the polypeptide consists of a clostridial neurotoxin light chain (L-chain) or fragment thereof; and/or a fragment of a clostridial neurotoxin heavy chain (H-chain).
7. The polypeptide for use, method or use according to any one of the preceding clauses, wherein the fragment of the clostridial neurotoxin H-chain comprises: a translocation domain (H_{N}) or fragment thereof; or a clostridial neurotoxin receptor binding domain (H_{C}) or fragment thereof.
8. The polypeptide for use, method or use according to any one of the preceding clauses, wherein the fragment of the clostridial neurotoxin H-chain comprises an H_{N} domain or fragment thereof.
9. The polypeptide for use, method or use according to any one of the preceding clauses, wherein the fragment of the clostridial neurotoxin H-chain consists of an H_{N} domain or fragment thereof.
10. The polypeptide for use, method or use according to any one of the preceding clauses, wherein the fragment of the clostridial neurotoxin H-chain comprises an H_{C} domain or fragment thereof.
11. The polypeptide for use, method or use according to any one of the preceding clauses, wherein the fragment of the clostridial neurotoxin H-chain consists of an H_{C} domain or fragment thereof.
12. The polypeptide for use, method or use according to any one of the preceding clauses, wherein the polypeptide lacks a C-terminal portion of a clostridial neurotoxin receptor binding domain (H_{cc}).
13. The polypeptide for use, method or use according to any one of the preceding clauses, wherein the polypeptide does not comprise both a clostridial neurotoxin H_{N} domain and H_{C} domain.
14. The polypeptide for use, method or use according to any one of the preceding clauses, wherein the polypeptide does not further comprise a non-clostridial catalytic domain.
15. The polypeptide for use, method or use according to any one of the preceding clauses, wherein the polypeptide comprises: a clostridial neurotoxin L-chain or fragment thereof, and H_{N} domain or fragment thereof.
16. The polypeptide for use, method or use according to any one of the preceding clauses, wherein the polypeptide consists of: a clostridial neurotoxin L-chain or fragment thereof, and H_{N} domain or fragment thereof.
17. The polypeptide for use, method or use according to any one of the preceding clauses, wherein the polypeptide consists of: a clostridial neurotoxin L-chain and H_{N} domain.
18. The polypeptide for use, method or use according to any one of the preceding clauses, wherein the polypeptide:
   a. is encoded by a nucleotide sequence having at least 70% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 70% sequence identity to any one of SEQ ID NOs: 4, 6, 8, 20, 22, 24, 28, 30, 32, 36, 38, 40, 42, 44, 46, 48 or 50.
19. The polypeptide for use, method or use according to any one of the preceding clauses, wherein the polypeptide:
   a. is encoded by a nucleotide sequence having at least 80% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 80% sequence identity to any one of SEQ ID NOs: 4, 6, 8, 20, 22, 24, 28, 30, 32, 36, 38, 40, 42, 44, 46, 48 or 50.
20. The polypeptide for use, method or use according to any one of the preceding clauses, wherein the polypeptide:
   a. is encoded by a nucleotide sequence having at least 90% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 90% sequence identity to any one of SEQ ID NOs: 4, 6, 8, 20, 22, 24, 28, 30, 32, 36, 38, 40, 42, 44, 46, 48 or 50.
21. The polypeptide for use, method or use according to any one of the preceding clauses, wherein the polypeptide:
   a. is encoded by a nucleotide sequence having at least 95% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 95% sequence identity to any one of SEQ ID NOs: 4, 6, 8, 20, 22, 24, 28, 30, 32, 36, 38, 40, 42, 44, 46, 48 or 50.
22. The polypeptide for use, method or use according to any one of the preceding clauses, wherein the polypeptide:
   a. is encoded by a nucleotide sequence having at least 99% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 99% sequence identity to any one of SEQ ID NOs: 4, 6, 8, 20, 22, 24, 28, 30, 32, 36, 38, 40, 42, 44, 46, 48 or 50.
23. The polypeptide for use, method or use according to any one of the preceding clauses, wherein the polypeptide:
   a. is encoded by a nucleotide sequence having at least 99.9% sequence identity to any one of SEQ ID NOs: 3, 5, 7, 19, 21, 23, 27, 29, 31, 35, 37, 39, 41, 43, 45, 47 or 49; or
   b. comprises (preferably consists of) a polypeptide sequence having at least 99.9% sequence identity to any one of SEQ ID NOs: 4, 6, 8, 20, 22, 24, 28, 30, 32, 36, 38, 40, 42, 44, 46, 48 or 50.
24. A polypeptide for use in promoting neuronal growth or neuronal repair to treat a neurological disorder in a subject, wherein the polypeptide comprises a catalytically inactive clostridial neurotoxin L-chain.
25. A method for promoting neuronal growth or neuronal repair to treat a neurological disorder in a subject, the method comprising administering a polypeptide to the subject, wherein the polypeptide comprises a catalytically inactive clostridial neurotoxin L-chain.
26. Use of a polypeptide comprising a catalytically inactive clostridial neurotoxin L-chain in the manufacture of a medicament for promoting neuronal growth or neuronal repair to treat a neurological disorder in a subject.
27. A polypeptide for use in promoting neuronal growth or neuronal repair to treat a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.
28. A method for promoting neuronal growth or neuronal repair to treat a neurological disorder in a subject, the method comprising administering a polypeptide to the subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41.
29. Use of a polypeptide in the manufacture of a medicament for promoting neuronal growth or neuronal repair to treat a neurological disorder in a subject, wherein the polypeptide comprises a polypeptide sequence having at least 70% sequence identity to SEQ ID NO: 42 and/or wherein the polypeptide comprises a polypeptide sequence that is encoded by a nucleotide sequence having at least 70% sequence identity to SEQ ID NO: 41
30. The polypeptide for use, method or use according to any one of clauses 27-29, wherein the polypeptide comprises (preferably consists of) a polypeptide sequence having at least 80% sequence identity to SEQ ID NO: 42 and/or wherein the polypeptide is encoded by a nucleotide sequence having at least 80% sequence identity to SEQ ID NO: 41.
31. The polypeptide for use, method or use according to any one of clauses 27-30, wherein the polypeptide comprises (preferably consists of) a polypeptide sequence having at least 90% sequence identity to SEQ ID NO: 42 and/or wherein the polypeptide is encoded by a nucleotide sequence having at least 90% sequence identity to SEQ ID NO: 41.
32. The polypeptide for use, method or use according to any one of clauses 27-31, wherein the polypeptide comprises (preferably consists of) a polypeptide sequence having at least 95% sequence identity to SEQ ID NO: 42 and/or wherein the polypeptide is encoded by a nucleotide sequence having at least 95% sequence identity to SEQ ID NO: 41.
33. The polypeptide for use, method or use according to any one of clauses 27-32, wherein the polypeptide comprises (preferably consists of) a polypeptide sequence having at least 99% sequence identity to SEQ ID NO: 42 and/or wherein the polypeptide is encoded by a nucleotide sequence having at least 99% sequence identity to SEQ ID NO: 41.
34. The polypeptide for use, method or use according to any one of clauses 27-33, wherein the polypeptide comprises (preferably consists of) a polypeptide sequence having at least 99.9% sequence identity to SEQ ID NO: 42 and/or wherein the polypeptide is encoded by a nucleotide sequence having at least 99.9% sequence identity to SEQ ID NO: 41.
35. The polypeptide for use, method or use according to any one of clauses 27-34, wherein the polypeptide comprises (preferably consists of) a polypeptide sequence having at least 70% sequence identity to SEQ ID NO: 61 or 65 and/or wherein the polypeptide is encoded by a nucleotide sequence having at least 70% sequence identity to SEQ ID NO: 60.
36. The polypeptide for use, method or use according to any one of clauses 27-35, wherein the polypeptide comprises (preferably consists of) a polypeptide sequence having at least 80% sequence identity to SEQ ID NO: 61 or 65 and/or wherein the polypeptide is encoded by a nucleotide sequence having at least 80% sequence identity to SEQ ID NO: 60.
37. The polypeptide for use, method or use according to any one of clauses 27-36, wherein the polypeptide comprises (preferably consists of) a polypeptide sequence having at least 90% sequence identity to SEQ ID NO: 61 or 65 and/or wherein the polypeptide is encoded by a nucleotide sequence having at least 90% sequence identity to SEQ ID NO: 60.
38. The polypeptide for use, method or use according to any one of clauses 27-37, wherein the polypeptide comprises (preferably consists of) a polypeptide sequence having at least 95% sequence identity to SEQ ID NO: 61 or 65 and/or wherein the polypeptide is encoded by a nucleotide sequence having at least 95% sequence identity to SEQ ID NO: 60.
39. The polypeptide for use, method or use according to any one of clauses 27-38, wherein the polypeptide comprises (preferably consists of) a polypeptide sequence having at least 99% sequence identity to SEQ ID NO: 61 or 65 and/or wherein the polypeptide is encoded by a nucleotide sequence having at least 99% sequence identity to SEQ ID NO: 60.
40. The polypeptide for use, method or use according to any one of clauses 27-39, wherein the polypeptide comprises (preferably consists of) a polypeptide sequence having at least 99.9% sequence identity to SEQ ID NO: 61 or 65 and/or wherein the polypeptide is encoded by a nucleotide sequence having at least 99.9% sequence identity to SEQ ID NO: 60.
41. A polypeptide for use in promoting neuronal growth or neuronal repair to treat a neurological disorder in a subject, wherein the polypeptide comprises a polypeptide sequence having at least 70% sequence identity to SEQ ID NO: 63 or 64.
42. A method for promoting neuronal growth or neuronal repair to treat a neurological disorder in a subject, the method comprising administering a polypeptide to the subject, wherein the polypeptide comprises a polypeptide sequence having at least 70% sequence identity to SEQ ID NO: 63 or 64.
43. Use of a polypeptide in the manufacture of a medicament for promoting neuronal growth or neuronal repair to treat a neurological disorder in a subject, wherein the polypeptide comprises a polypeptide sequence having at least 70% sequence identity to SEQ ID NO: 63 or 64.
44. The polypeptide for use, method or use according to any one of clauses 41-43, wherein the polypeptide comprises (preferably consists of) a polypeptide sequence having at least 80% sequence identity to SEQ ID NO: 63 or 64.
45. The polypeptide for use, method or use according to any one of clauses 41-44, wherein the polypeptide comprises (preferably consists of) a polypeptide sequence having at least 90% sequence identity to SEQ ID NO: 63 or 64.
46. The polypeptide for use, method or use according to any one of clauses 41-45, wherein the polypeptide comprises (preferably consists of) a polypeptide sequence having at least 95% sequence identity to SEQ ID NO: 63 or 64.
47. The polypeptide for use, method or use according to any one of clauses 41-46, wherein the polypeptide comprises (preferably consists of) a polypeptide sequence having at least 99% sequence identity to SEQ ID NO: 63 or 64.
48. The polypeptide for use, method or use according to any one of clauses 41-47, wherein the polypeptide comprises (preferably consists of) a polypeptide sequence having at least 99.9% sequence identity to SEQ ID NO: 63 or 64.
49. The polypeptide for use, method or use according to any one of the preceding clauses, wherein the polypeptide does not comprise a native clostridial neurotoxin H-chain.
50. The polypeptide for use, method or use according to any one of the preceding clauses, wherein the polypeptide is neurotrophic.
51. The polypeptide for use, method or use according to any one of the preceding clauses, wherein the polypeptide promotes neuronal growth and/or neuronal repair.
52. The polypeptide for use, method or use according to any one of the preceding clauses, wherein the neurological disorder is a disorder that can be treated by promoting neuronal growth and/or repair.
53. The polypeptide for use, method or use according to any one of the preceding clauses, wherein the neurological disorder is a neuronal injury, a neurodegenerative disorder, a sensory disorder or an autonomic disorder.
54. The polypeptide for use, method or use according to any one of the preceding clauses, wherein the neurological disorder is a neuronal injury selected from: a nerve trauma (e.g. resulting from scarring and/or from a bone fracture), a neuropathy (e.g. peripheral neuropathy), a spinal cord injury (e.g. including paralysis), a nerve section, a brain injury (e.g. traumatic brain injury), a non-traumatic injury (e.g. stroke or spinal cord infarction), and an injury to the brachial plexus, e.g. Erb's palsy or Klumpke's palsy.
55. The polypeptide for use, method or use according to any one of the preceding clauses, wherein the neurological disorder is a neurodegenerative disorder selected from: Alzheimer's disease, Parkinson's disease, Parkinson's disease related disorders, motor neuron disease, peripheral neuropathy, motor neuropathy, prion disease, Huntington's disease, spinocerebellar ataxia, spinal muscular atrophy, monomelic amyotrophy, Friedreich's ataxia, Hallervorden-Spatz disease, and frontotemporal lobar degeneration.
56. The polypeptide for use, method or use according to any one of the preceding clauses, wherein the polypeptide promotes growth or repair of a motor neuron.
57. The polypeptide for use, method or use according to any one of the preceding clauses, wherein the polypeptide is a modified clostridial neurotoxin, such as a chimeric clostridial neurotoxin or a hybrid clostridial neurotoxin.
58. The polypeptide for use, method or use according to any one of clauses 24-34 or 49-57, wherein the polypeptide is catalytically inactive and:
   a. is encoded by a nucleotide sequence having at least 70% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
59. The polypeptide for use, method or use according to any one of clauses 24-34 or 49-58, wherein the polypeptide is catalytically inactive and:
   a. is encoded by a nucleotide sequence having at least 80% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 80% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
60. The polypeptide for use, method or use according to any one of clauses 24-34 or 49-59, wherein the polypeptide is catalytically inactive and:
   a. is encoded by a nucleotide sequence having at least 90% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 90% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
61. The polypeptide for use, method or use according to any one of clauses 24-34 or 49-60, wherein the polypeptide is catalytically inactive and:
   a. is encoded by a nucleotide sequence having at least 95% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 95% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
62. The polypeptide for use, method or use according to any one of clauses 24-34 or 49-61, wherein the polypeptide is catalytically inactive and:
   a. is encoded by a nucleotide sequence having at least 99% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 99% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
63. The polypeptide for use, method or use according to any one of clauses 24-34 or 49-62, wherein the polypeptide is catalytically inactive and:
   a. is encoded by a nucleotide sequence having at least 99.9% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
   b. comprises (preferably consists of) a polypeptide sequence having at least 99.9% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.
64. The polypeptide for use, method or use according to any one of clauses 24-26 or 49-63, wherein the polypeptide:
   a. is encoded by a nucleotide sequence having at least 70% sequence identity to any one of SEQ ID NOs: 1, 9, 11, 13, 15, 17, 25 or 33; or
   b. comprises (preferably consists of) a polypeptide sequence having at least 70% sequence identity to any one of SEQ ID NOs: 2, 10, 12, 14, 16, 18, 26, 34, 64 or 65.
65. The polypeptide for use, method or use according to any one of clauses 24-26 or 49-64, wherein the polypeptide:
   a. is encoded by a nucleotide sequence having at least 80% sequence identity to any one of SEQ ID NOs: 1, 9, 11, 13, 15, 17, 25 or 33; or
   b. comprises (preferably consists of) a polypeptide sequence having at least 80% sequence identity to any one of SEQ ID NOs: 2, 10, 12, 14, 16, 18, 26, 34, 64 or 65.
66. The polypeptide for use, method or use according to any one of clauses 24-26 or 49-65, wherein the polypeptide:
   a. is encoded by a nucleotide sequence having at least 90% sequence identity to any one of SEQ ID NOs: 1, 9, 11, 13, 15, 17, 25 or 33; or
   b. comprises (preferably consists of) a polypeptide sequence having at least 90% sequence identity to any one of SEQ ID NOs: 2, 10, 12, 14, 16, 18, 26, 34, 64 or 65.
67. The polypeptide for use, method or use according to any one of clauses 24-26 or 49-66, wherein the polypeptide:
   a. is encoded by a nucleotide sequence having at least 95% sequence identity to any one of SEQ ID NOs: 1, 9, 11, 13, 15, 17, 25 or 33; or
   b. comprises (preferably consists of) a polypeptide sequence having at least 95% sequence identity to any one of SEQ ID NOs: 2, 10, 12, 14, 16, 18, 26, 34, 64 or 65.
68. The polypeptide for use, method or use according to any one of clauses 24-26 or 49-67, wherein the polypeptide:
   a. is encoded by a nucleotide sequence having at least 99% sequence identity to any one of SEQ ID NOs: 1, 9, 11, 13, 15, 17, 25 or 33; or
   b. comprises (preferably consists of) a polypeptide sequence having at least 99% sequence identity to any one of SEQ ID NOs: 2, 10, 12, 14, 16, 18, 26, 34, 64 or 65.
69. The polypeptide for use, method or use according to any one of clauses 24-26 or 49-68, wherein the polypeptide:
   a. is encoded by a nucleotide sequence having at least 99.9% sequence identity to any one of SEQ ID NOs: 1, 9, 11, 13, 15, 17, 25 or 33; or
   b. comprises (preferably consists of) a polypeptide sequence having at least 99.9% sequence identity to any one of SEQ ID NOs: 2, 10, 12, 14, 16, 18, 26, 34, 64 or 65.
70. The polypeptide for use, method or use according to any one of the preceding clauses, wherein the polypeptide is administered at, or near to, a site of injury, preferably wherein the polypeptide is administered intrathecally.
71. The polypeptide for use, method or use according to any one of the preceding clauses, wherein the polypeptide does not further comprise a domain that binds to a cellular receptor.
72. The polypeptide for use, method or use according to any one of the preceding clauses, wherein the polypeptide lacks a functional H_{C} domain of a clostridial neurotoxin and also lacks any functionally equivalent exogenous ligand Targeting Moiety (TM).
73. The polypeptide for use, method or use according to any one of the preceding clauses, wherein the polypeptide is not expressed in a cell of the subject.
74. The polypeptide for use, method or use according to any one of the preceding clauses, wherein the clostridial sequences of the polypeptide consist of a clostridial neurotoxin light chain (L-chain) or fragment thereof; and/or a fragment of a clostridial neurotoxin heavy chain (H-chain).
75. The polypeptide for use, method or use according to any one of the preceding clauses, wherein the polypeptide further comprises one or more non-clostridial neurotoxin sequences.
76. The polypeptide for use, method or use according to clause 75, wherein the one or more non-clostridial neurotoxin sequences do not bind to a cellular receptor.
77. The polypeptide for use, method or use according to clause 75 or 76, wherein the one or more non-clostridial neurotoxin sequences do not comprise a ligand for a cellular receptor.
78. The polypeptide for use, method or use according to any one of clauses 1-40 or 49-77, wherein the polypeptide is a modified BoNT/A or fragment thereof comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.
79. The polypeptide for use, method or use according to any one of clauses 1-26 or 41-77, wherein the polypeptide is a chimeric BoNT comprising a BoNT/A light-chain and translocation domain, and a BoNT/B receptor binding domain (H_{C} domain).

1. (canceled)

2. A method for treating a neurological disorder in a subject, the method comprising administering a polypeptide to the subject, wherein the polypeptide comprises:
(a) a clostridial neurotoxin light chain (L-chain) or fragment thereof, wherein the clostridial neurotoxin L-chain or fragment thereof is catalytically inactive; or
(b) a fragment of a clostridial neurotoxin heavy chain (H-chain), wherein the fragment of the clostridial neurotoxin H-chain comprises a clostridial neurotoxin receptor binding domain (H_{C} domain) or fragment thereof, and wherein the polypeptide does not comprise both a clostridial neurotoxin translocation domain (H_{N} domain) and a clostridial neurotoxin H_{C} domain.

3-5. (canceled)

6. The method according to claim 2, wherein the polypeptide consists of: the clostridial neurotoxin L-chain or fragment thereof, wherein the clostridial neurotoxin L-chain or fragment thereof is catalytically inactive; the fragment of the clostridial neurotoxin heavy chain (H-chain); or a combination thereof.

7-10. (canceled)

11. The method according to claim 2, wherein the fragment of the clostridial neurotoxin H-chain consists of an H_{C} domain or fragment thereof.

12. The method according to claim 2, wherein the polypeptide lacks a C-terminal portion of a clostridial neurotoxin receptor binding domain (H_{cc}).

13. (canceled)

14. The method according to claim 2, wherein the polypeptide does not further comprise a non-clostridial catalytic domain.

15. The method according to claim 2, wherein the polypeptide comprises: the clostridial neurotoxin L-chain or fragment thereof, wherein the clostridial neurotoxin L-chain or fragment thereof is catalytically inactive; and an H_{N} domain or fragment thereof.

16. The method according to claim 2, wherein the polypeptide consists of: the clostridial neurotoxin L-chain or fragment thereof, wherein the clostridial neurotoxin L-chain or fragment thereof is catalytically inactive; and an H_{N} domain or fragment thereof.

17. The method according to claim 2, wherein the polypeptide consists of: the clostridial neurotoxin L-chain, wherein the clostridial neurotoxin L-chain is catalytically inactive; and an H_{N} domain.

18-48. (canceled)

49. The method according to claim 2, wherein the polypeptide does not comprise a native clostridial neurotoxin H-chain.

50. The method according to claim 2, wherein the polypeptide is neurotrophic.

51-52. (canceled)

53. The method according to claim 2, wherein the neurological disorder is a neuronal injury, a neurodegenerative disorder, a sensory disorder or an autonomic disorder.

54. The method according to claim 2, wherein the neurological disorder is a neuronal injury selected from: a nerve trauma, a neuropathy, a spinal cord injury, a nerve section, a brain injury, a non-traumatic injury, and an injury to the brachial plexus.

55. The method according to claim 2, wherein the neurological disorder is a neurodegenerative disorder selected from: Alzheimer's disease, Parkinson's disease, Parkinson's disease related disorders, motor neuron disease, peripheral neuropathy, motor neuropathy, prion disease, Huntington's disease, spinocerebellar ataxia, spinal muscular atrophy, monomelic amyotrophy, Friedreich's ataxia, Hallervorden-Spatz disease, and frontotemporal lobar degeneration.

56. The method according to claim 2, wherein the polypeptide promotes growth or repair of a motor neuron.

57. The method according to claim 2, wherein the polypeptide is a modified clostridial neurotoxin.

58-59. (canceled)

60. The method according to claim 2, wherein the polypeptide:
(a) is encoded by a nucleotide sequence having at least 90% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60; or
(b) comprises a polypeptide sequence having at least 90% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, or 63.

61-69. (canceled)

70. The method according to claim 2, wherein the polypeptide is administered at, or near to, a site of injury.

71-78. (canceled)

79. The method according to claim 2, wherein the polypeptide is a chimeric BoNT comprising a BoNT/A light-chain and translocation domain, and a BoNT/B receptor binding domain (H_{C} domain).

80. The method according to claim 57, wherein the modified clostridial neurotoxin is a chimeric clostridial neurotoxin or a hybrid clostridial neurotoxin.

81. A method for promoting neuronal growth or neuronal repair to treat a neurological disorder in a subject, the method comprising administering a polypeptide to the subject, wherein the polypeptide consists of:
(a) a clostridial neurotoxin light chain (L-chain); or
(b) a clostridial neurotoxin L-chain and a clostridial neurotoxin translocation domain (H_{N} domain).


SEQUENCE LISTING (76 sequences)

SEQ ID NO: 1 - DNA, 3888 bp, Artificial Sequence
  Nucleotide Sequence of rBoNT/A(0)
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatcaactgatctacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Artificial Sequence
  Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 2634 bp, Artificial Sequence
  Nucleotide Sequence of rLHN/A
atggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctgacgatgacgataaaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaagcacaccatcatcaccaccatcaccatcaccat

SEQ ID NO: 4 - protein, 878 aa, Artificial Sequence
  Polypeptide Sequence of rLHN/A
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEAHHHHHHHHHH

SEQ ID NO: 5 - DNA, 1329 bp, Artificial Sequence
  Nucleotide Sequence of rL/A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgggtctagaagcacaccatcatcaccaccatcaccatcaccat

SEQ ID NO: 6 - protein, 443 aa, Artificial Sequence
  Polypeptide Sequence of rL/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLGLEAHHHHHHHHHH

SEQ ID NO: 7 - DNA, 1299 bp, Artificial Sequence
  Nucleotide Sequence of rHC/A
atgcatcatcaccatcaccacaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 433 aa, Artificial Sequence
  Polypeptide Sequence of rHC/A
MHHHHHHKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 9 - DNA, 3873 bp, Artificial Sequence
  Nucleotide Sequence of rBoNT/B(0)
atgccggtgacgattaacaacttcaactacaacgacccgattgacaacaacaacattatcatgatggaaccgccgtttgcacgcggcacgggccgttattacaaagcgtttaaaatcaccgatcgtatttggattatcccggaacgctacacgtttggttataaaccggaagacttcaacaaaagctctggcatcttcaaccgtgatgtttgcgaatactacgatccggactacctgaacaccaacgataagaaaaacatttttctgcaaacgatgatcaaactgttcaatcgcattaaaagcaaaccgctgggtgaaaaactgctggaaatgattatcaatggcattccgtatctgggtgatcgtcgcgtgccgctggaagaatttaacaccaatatcgcgagtgttacggtcaacaaactgatttccaatccgggtgaagtcgaacgtaaaaaaggcatcttcgccaacctgatcatcttcggcccgggtccggtgctgaacgaaaatgaaaccattgatatcggtattcagaaccattttgcctcacgcgaaggcttcggcggtattatgcaaatgaaattttgcccggaatatgtgtcggttttcaacaatgttcaggaaaacaaaggtgcaagcatctttaatcgtcgcggctatttctctgatccggctctgatcctgatgcaccaactgatttatgtgctgcacggcctgtatggtatcaaagtggatgacctgccgatcgttccgaacgagaaaaaatttttcatgcagagcaccgacgcaattcaagctgaagaactgtatacgtttggcggtcaggacccgtctattatcaccccgagcaccgacaaaagcatctacgataaagtgctgcaaaactttcgtggcattgttgaccgcctgaataaagtcctggtgtgtatctctgatccgaacatcaacatcaacatctacaaaaacaaattcaaagacaaatacaaattcgttgaagattctgaaggcaaatatagtattgacgtcgaatcctttgataaactgtacaaaagtctgatgttcggtttcaccgaaacgaacatcgcggaaaactacaaaatcaaaacccgcgcctcctatttcagcgactctctgccgccggttaaaatcaaaaatctgctggataacgaaatttatacgatcgaagaaggtttcaacatcagcgataaagacatggaaaaagaataccgtggccagaataaagcaatcaacaaacaggcgtatgaagaaattagtaaagaacatctggcggtctacaaaattcagatgtgcaaatccgtgaaagccccgggtatttgtatcgatgttgacaatgaagacctgtttttcatcgccgataaaaacagtttttccgatgacctgtcaaaaaatgaacgcatcgaatacaacacccaatcgaactacatcgaaaacgatttcccgatcaacgaactgattctggatacggacctgattagtaaaatcgaactgccgtcagaaaacaccgaatcgctgacggactttaatgttgatgtcccggtgtatgaaaaacagccggcaattaagaaaatttttaccgatgaaaacacgatcttccagtacctgtacagccaaacctttccgctggacattcgcgatatctctctgacgagttcctttgatgacgcactgctgttcagcaacaaagtgtactcctttttctcaatggattacatcaaaaccgctaacaaagtggttgaagcgggcctgtttgccggttgggtgaaacagatcgttaacgatttcgtcatcgaagccaacaaaagtaacacgatggataaaattgctgatatctccctgattgtcccgtatattggcctggcactgaatgtgggtaacgaaacggcgaaaggcaattttgaaaacgccttcgaaattgcaggcgcttcaatcctgctggaatttattccggaactgctgatcccggtcgtgggtgcgttcctgctggaatcttacatcgacaacaaaaacaaaatcatcaaaaccattgataacgcgctgacgaaacgtaacgaaaaatggtcagatatgtacggcctgattgttgcccagtggctgagcaccgtcaacacgcaattttacaccatcaaagaaggtatgtacaaagcgctgaattatcaggcgcaagccctggaagaaatcatcaaataccgctacaacatctacagcgaaaaagaaaaatctaacatcaacatcgactttaatgatatcaacagcaaactgaacgaaggtatcaaccaggcaatcgataacatcaacaacttcatcaacggctgctcagtgtcgtatctgatgaagaaaatgatcccgctggctgttgaaaaactgctggattttgacaacaccctgaagaaaaacctgctgaactacatcgatgaaaacaaactgtacctgatcggctcagccgaatacgaaaaatcgaaagtgaacaaatacctgaaaaccatcatgccgtttgacctgagtatttacaccaacgatacgatcctgatcgaaatgttcaacaaatacaactccgaaattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaagagaaactgttcctggccccgattagcgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 10 - protein, 1291 aa, Artificial Sequence
  Polypeptide Sequence of rBoNT/B(0)
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - DNA, 3873 bp, Artificial Sequence
  Nucleotide Sequence of rBoNT/C(0)
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgggcacgctgaacaatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaa

SEQ ID NO: 12 - protein, 1291 aa, Artificial Sequence
  Polypeptide Sequence of rBoNT/C(0)
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMGTLNNAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 13 - DNA, 3756 bp, Artificial Sequence
  Nucleotide Sequence of rBoNT/E(0)
atgccgaaaatcaactctttcaactacaacgacccggttaacgaccgtaccatcctgtatatcaaaccgggtggttgccaggagttctacaaatctttcaacatcatgaaaaacatctggatcatcccggaacgtaacgttatcggtaccaccccgcaggacttccacccgccgacctctctgaaaaacggtgactcttcttactacgacccgaactacctccagtctgacgaagaaaaagaccgtttcctgaaaatcgttaccaaaatcttcaaccgtatcaacaacaacctgtctggtggtatcctgctggaagaactgtctaaagctaacccgtacctgggtaacgacaacaccccggacaaccagttccacatcggtgacgcttctgctgttgaaatcaaattctctaacggttctcaggacatcctgctgccgaacgttatcatcatgggtgctgaaccggacctgttcgaaaccaactcttctaacatctctctgcgtaacaactacatgccgtctaaccacggtttcggttctatcgctatcgttaccttctctccggaatactctttccgtttcaacgacaacagcatgaacgagttcatccaggacccggctctgaccctgatgcaccaactgatctactctctgcacggtctgtacggtgctaaaggtatcaccaccaaatacaccatcacccagaaacagaacccgctgatcaccaacatccgtggtaccaacatcgaagagttcctgaccttcggtggtaccgacctgaacatcatcacctctgctcagtctaacgacatctacaccaacctgctggctgactacaaaaaaatcgcttctaaactgtctaaagttcaggtttctaacccgctgctgaacccgtacaaagacgttttcgaagctaaatacggtctggacaaagacgcttctggtatctactctgttaacatcaacaaattcaacgacatcttcaaaaaactgtactctttcaccgagttcgacctggcgaccaaattccaggttaaatgccgtcagacctacatcggtcagtacaaatacttcaaactgtctaacctgctgaacgactctatctacaacatctctgaaggttacaacatcaacaacctgaaagttaacttccgtggtcagaacgctaacctgaacccgcgtatcatcaccccgatcaccggtcgtggtctggttaaaaaaatcatccgtttctgcaagaatattgtaagcgttaaaggaataagaaaaagtatctgcatcgaaatcaacaacggtgaactgttcttcgttgcttctgaaaactcttacaacgacgacaacatcaacaccccgaaagaaatcgacgacaccgttacctctaacaacaactacgaaaacgacctggaccaggttatcctgaacttcaactctgaatctgctccgggtctgtctgacgaaaaactgaacctgaccatccagaacgacgcttacatcccgaaatacgactctaacggtacctctgacatcgaacagcacgacgttaacgaactgaacgttttcttctacctggacgctcagaaagttccggaaggtgaaaacaacgttaacctgacctcttctatcgacaccgctctgctggaacagccgaaaatctacaccttcttctcttctgagttcatcaacaacgttaacaaaccggttcaggctgctctgttcgtttcttggattcagcaggttctggttgacttcaccaccgaagctaaccagaaatctaccgttgacaaaatcgctgacatctctatcgttgttccgtacatcggtctggctctgaacatcggtaacgaagctcagaaaggtaacttcaaagacgctctggaactgctgggtgctggtatcctgctggagttcgaaccggaactgctgatcccgaccatcctggttttcaccatcaaatctttcctgggttcttctgacaacaaaaacaaagttatcaaagctatcaacaacgctctgaaagaacgtgacgaaaaatggaaagaagtttactctttcatcgtttctaactggatgaccaaaatcaacacccagttcaacaaacgtaaagaacagatgtaccaggctctccagaaccaggttaacgctatcaaaaccatcatcgaatctaaatacaactcttacaccctggaagaaaaaaacgaactgaccaacaaatacgacatcaaacagatcgaaaacgaactgaaccagaaagtttctatcgctatgaacaacatcgaccgtttcctgaccgaatcttctatctcttacctgatgaaactcatcaacgaagttaaaatcaacaaactgcgtgaatacgacgaaaacgttaaaacctacctgctgaactacatcatccagcacggttctatcctgggtgaatctcagcaggaactgaactctatggttaccgacaccctgaacaactctatcccgttcaaactgtcttcttacaccgacgacaaaatcctgatctcttacttcaacaaattctttaaacgcattaagagttcatcggttctgaatatgcggtacaaaaatgataaatatgtcgatacttctggatatgatagcaatatcaacattaacggcgacgtgtataaatatccgacaaataaaaaccagtttgggatatataacgacaagctgtcggaggtcaatatttctcaaaacgactatatcatttacgataataaatataaaaactttagcattagtttttgggttcgtatacctaattatgacaataaaattgtaaatgtgaataacgagtataccattataaactgtatgcgcgacaataacagtggttggaaggtatcgctgaaccataatgagattatctggaccctgcaggataatgcaggtataaaccagaaactggcttttaactatggaaacgcaaatgggatctcagattacattaataaatggatttttgttaccattacgaacgatcgcttaggcgactcaaaactttatattaatggcaatctgatagatcagaaatcaatcttaaatttgggcaatattcatgtctctgataacatcttgttcaagatcgttaattgcagttacactcgttatattggcattcgttactttaatatcttcgataaagaactggacgagacggaaatccagactctgtattcaaacgagcccaatactaatatattgaaagatttttggggtaactatcttttatatgataaagaatactatctcctgaatgtattgaagccaaacaatttcatagatagacgcaaggatagcacattaagtatcaacaatatcagatctactatactgttagcaaatcgcctctactccggtattaaagtgaagattcagcgggttaataactccagtaccaatgataatctggtccgtaagaacgatcaggtatacatcaatttcgtcgcgagcaaaactcatctcttcccgctttacgccgatacagctacgacaaacaaggaaaaaaccataaaaatttccagctccggaaacagattcaatcaagtagttgtaatgaactctgtgggtaataattgtacgatgaactttaagaataacaatgggaacaatattggacttttgggcttcaaagccgacacagtggtggcgtccacctggtattacacgcacatgcgggaccatacgaattcgaacggttgcttctggaactttatctcggaagaacacgggtggcaagaaaaa

SEQ ID NO: 14 - protein, 1252 aa, Artificial Sequence
  Polypeptide Sequence of rBoNT/E(0)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHQLIYSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 15 - DNA, 3834 bp, Artificial Sequence
  Nucleotide Sequence of rBoNT/F(0)
atgccggtggtcatcaacagcttcaactacaacgacccagtaaacgacgacacgatcctgtatatgcaaatcccgtatgaagagaagagcaagaagtactataaggcctttgaaatcatgcgcaatgtgtggattattccggagcgtaatacgattggtactgacccaagcgacttcgatccacctgcgtctttggaaaacggctcgtccgcatattacgacccgaattacctgaccaccgatgcggagaaagatcgttatttgaaaaccaccatcaagctgttcaaacgcattaacagcaatccggcaggtgaggtcctgctgcaagagattagctacgcaaagccttatctgggtaatgagcatacgcctattaacgagtttcacccggttacccgcactaccagcgttaacatcaagtcctcgaccaacgtgaagtctagcattatcctgaacctgctggttctgggtgccggtccggacatcttcgaaaactctagctacccggtgcgtaaactgatggatagcggcggtgtttatgacccgagcaatgacggttttggcagcatcaatatcgtgacgtttagcccggagtacgagtacaccttcaatgatatcagcggtggttacaattcttctaccgagagcttcatcgccgacccggcgatcagcctggcacaccaactgatctatgcattgcatggcttgtacggtgcccgtggtgtgacgtataaagagactatcaaggttaagcaggcacctctgatgattgcggaaaagccgattcgcctggaagagttcctgaccttcggcggtcaagatttgaacatcattacctcggccatgaaagagaaaatctataacaatttgctggccaactatgaaaagattgcaacgcgcttgtctcgtgttaactccgctccgccggaatacgacattaatgagtacaaagactactttcaatggaaatatggcctggacaaaaatgcggatggttcttataccgtgaatgaaaacaaattcaatgaaatctacaagaaactgtacagcttcaccgaaatcgatctggcgaacaagttcaaagtcaaatgtcgtaatacctacttcatcaaatatggcttcctgaaagtcccgaacctgctggacgatgacatctataccgtcagcgaaggcttcaacatcggcaatctggccgtgaataatcgtggtcagaacatcaaactgaatccgaaaatcattgactccatcccagacaagggcctggttgagaaaatcgtgaagttctgcaaaagcgttattccgcgtaaaggtacgaaagcaccgcctcgcctgtgcattcgcgttaacaaccgtgagttgttctttgtggcatctgaaagcagctacaacgagaacgacatcaacacccctaaagaaattgatgataccacgaacctgaataacaattatcgcaacaatctggacgaggtgatcctggattacaattcggaaaccattccgcaaattagcaatcagacgctgaacaccctggttcaggacgatagctacgttccgcgttacgactccaatggtactagcgagattgaagaacacaacgtagtggacttgaacgttttcttttatctgcacgcccagaaggttccggagggcgaaaccaatattagcctgaccagctcgatcgacaccgcgctgtctgaggagagccaagtctacacctttttcagcagcgagtttatcaacactattaacaagccagttcatgctgcattgtttatctcttggattaaccaggtgattcgcgactttacgacggaggcgacccagaagtctaccttcgacaaaattgcagacatctccctggtcgtcccatacgtcggcctggcgttgaatattggcaatgaagttcaaaaagagaacttcaaagaagcgttcgagctgctgggtgcaggcatcctgctggagttcgtgccggaactgttgatcccgaccatcctggtgttcaccattaagagcttcattggatcctccgagaataagaacaagatcatcaaggcgatcaataacagcctgatggagcgtgaaacgaagtggaaagaaatctatagctggattgttagcaattggctgactcgtattaacacgcaattcaacaagcgtaaagagcaaatgtaccaagccctgcaaaaccaagttgacgccatcaaaacggtaattgaatacaagtacaacaattacacgagcgatgagcgcaaccgcctggaaagcgaatacaacatcaacaacattcgcgaagaattgaacaagaaagtgagcctggcgatggagaacattgagcgttttatcaccgaaagcagcatcttttacctgatgaaattgattaatgaggcgaaagtctcgaaactgcgtgagtacgacgaaggtgtgaaagagtatctgctggattacattagcgagcaccgtagcatcttgggtaactcggttcaggagctgaacgatctggtgacctctaccctgaacaatagcatcccgttcgaactgagcagctataccaatgacaagattctgattctgtatttcaataaactgtataagaagatcaaggataacagcattctggatatgcgttacgaaaacaataagtttatcgacatttctggttacggcagcaacatttccatcaatggcgatgtctacatctacagcaccaatcgcaaccagttcggcatctactctagcaaaccgagcgaagttaacatcgcacagaacaatgatattatttataacggtcgttatcaaaacttctctatcagcttttgggtccgtatcccgaagtacttcaataaagtcaatctgaataatgaatacacgatcatcgactgcattcgcaataacaacagcggttggaaaatcagcctgaattacaacaaaattatttggaccctgcaagatacggcgggtaacaatcagaaactggtgtttaactacacgcaaatgatcagcatttctgactatatcaacaagtggatctttgttaccatcaccaataatcgtctgggcaatagccgtatttacatcaacggtaacctgattgatgagaaaagcatcagcaacctgggcgatattcacgtcagcgacaacattctgttcaaaattgttggttgtaacgatacccgttacgtcggcatccgttatttcaaggttttcgatacggagctgggtaaaacggaaatcgaaacgttgtactccgatgaaccagatccgagcattctgaaggacttttggggtaactacttgctgtacaataaacgttactatctgctgaatctgttgcgcaccgacaagagcattacccaaaacagcaatttcctgaacattaatcagcaacgcggcgtataccaaaaaccgaacatcttcagcaatacgcgcctgtatactggtgttgaagtgatcattcgtaagaacggtagcaccgacattagcaacacggacaatttcgtccgtaagaatgacctggcgtacattaacgtcgtggaccgtgatgtcgagtatcgtctgtacgcagacatcagcattgcgaaaccggaaaagattatcaagctgatccgtaccagcaacagcaacaacagcctgggtcagatcattgtgatggacagcattggtaataactgcacgatgaacttccagaacaacaatggtggtaatatcggtctgctgggttttcacagcaataatctggttgcttccagctggtactacaataacattcgtaaaaacacgtctagcaatggttgtttttggagctttatcagcaaagagcacggctggcaagaaaat

SEQ ID NO: 16 - protein, 1278 aa, Artificial Sequence
  Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 17 - DNA, 3942 bp, Artificial Sequence
  Nucleotide Sequence of rBoNT/A(0) (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacaccagctgatttatgccggtcatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaacctttaccgaatacattaagaacatcatcaacaccagcattctgaacctgcgttatgaaagcaatcatctgattgatctgagccgttatgccagcaaaatcaatataggcagcaaggttaacttcgacccgattgacaaaaatcagatacagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggctggaaagttagcctgaattatggcgaaattatctggaccctgcaggatacccaagaaatcaaacagcgtgtggttttcaaatacagccagatgattaatatcagcgactatatcaaccgctggatttttgtgaccattaccaataatcgcctgaataacagcaagatctatattaacggtcgtctgattgaccagaaaccgattagtaatctgggtaatattcatgcgagcaacaacatcatgtttaaactggatggttgtcgtgatacccatcgttatatttggatcaagtacttcaacctgttcgataaagagttgaacgaaaaagaaattaaagacctgtatgataaccagagcaacagcggtattctgaaggatttttggggagattatctgcagtatgacaaaccgtattatatgctgaatctgtacgacccgaataaatacgtggatgtgaataatgttggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctggaaaacctgtattttcaaggtgcaagtcatcatcaccatcaccaccatcattaa

SEQ ID NO: 18 - protein, 1313 aa, Artificial Sequence
  Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLENLYFQGASHHHHHHHH

SEQ ID NO: 19 - DNA, 2649 bp, Artificial Sequence
  Nucleotide Sequence of rLHN/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaaccgaaaatctgtatttccagggtgcaagtcatcatcaccatcaccaccatcattaa

SEQ ID NO: 20 - protein, 882 aa, Artificial Sequence
  Polypeptide Sequence of rLHN/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTENLYFQGASHHHHHHHH

SEQ ID NO: 21 - DNA, 1323 bp, Artificial Sequence
  Nucleotide Sequence of rHC/A (His-tagged)
atgcatcatcaccatcaccacgaaaatctatacttccaaggaaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctgtaa

SEQ ID NO: 22 - protein, 440 aa, Artificial Sequence
  Polypeptide Sequence of rHC/A (His-tagged)
MHHHHHHENLYFQGKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 23 - DNA, 1326 bp, Artificial Sequence
  Nucleotide Sequence of rLC/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgtttgaagagaatctgtatttccagggtgcaagtcatcatcaccatcaccaccatcattaa

SEQ ID NO: 24 - protein, 441 aa, Artificial Sequence
  Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEENLYFQGASHHHHHHHH

SEQ ID NO: 25 - DNA, 3918 bp, Artificial Sequence
  Nucleotide Sequence of rBoNT/FA(0) (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcaccagctgatttatgttctgcatggtctgtatggcacctatatcaccgaatacaaaattaccccgaatgtggttcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattaccgaaagcagcattgcctatctgatcaatatcatcaacaacgaaacgatccagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaactgaagtacaactgcattctgaacatcaaatatgagatggatcgtgacaaactggttgatagcagcggttatcgtagccgtatcaatattggtacaggcgtcaaatttagcgagatcgataaaaatcaagtgcagctgagcaatctggaatccagcaaaattgaagtcattctgaataacggcgtcatctataacagcatgtatgaaaacttttcgaccagcttttggattcgcattccgaaatactttcgcaacatcaataacgagtacaagatcatcagctgtatgcagaataatagcggttgggaagtgagcctgaattttagcaatatgaactcgaaaatcatctggaccctgcaggataccgaaggtatcaaaaaaaccgttgtgtttcagtacacccagaacattaacattagcgactatatcaaccgctggatctttgtgaccattacaaataatcgtctgagcaacagcaaaatctacattaatggtcgcctgatcaacgaagaaagcattagcgatctgggtaatatccatgccagcaacaacattatgtttaaactggatggttgccgtgatccgcatcgttatatctggattaaatactttaacctgtttgacaaagagctgaacaagaaagaaattaaagatctgtacgacaaccagagcaatagcggtattctgaaagatttctggggtgattatctgcagtatgacaaaccgtattatatgctgaatctgtatgacccgaataagtatctggatgtgaataatgttggcatccgtggctatatgtatctgaaaggtccgcgtggtcgtattgtgaccaccaacatttatctgaatagcaccctgtatatgggcaccaaattcatcattaagaaatatgccagcggcaacaaagataacattgtgcgtaataatgatcgcgtgtatattaacgtggtggtgaagaataaagaatatcgcctggcaaccaatgcaagccaggcaggcgttgaaaaaattctgagcgcagttgaaatcccggatgttggtaatctgagccaggttgttgtgatgaaaagcgaaaatgatcagggcattcgcaacaagtgtaaaatgaatctgcaagacaataacggcaacgatattggctttatcggctttcaccagtttaataacattgcaaaactggtggccagcaactggtataaccgtcagattggtaaagcaagccgtacctttggttgtagctgggaatttatcccggttgatgatggttggggtgaaagcagcctggaaaatctgtatttccagggtgccagtcatcatcaccaccatcaccatcactga

SEQ ID NO: 26 - protein, 1305 aa, Artificial Sequence
  Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHQLIYVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 27 - DNA, 2634 bp, Artificial Sequence
  Nucleotide Sequence of rLHN/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattacagaaagcagcattgcctacctgatcaatatcatcaacaacgaaaccattcagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaagagaacctgtatttccagggtgccagtcatcatcaccaccatcaccatcactga

SEQ ID NO: 28 - protein, 877 aa, Artificial Sequence
  Polypeptide Sequence of rLHN/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGEENLYFQGASHHHHHHHH

SEQ ID NO: 29 - DNA, 1341 bp, Artificial Sequence
  Nucleotide Sequence of rHC/FA (His-tagged)
atgctgaagtataactgcatcctgaacatcaaatatgagatggatcgtgataaactggttgatagcagcggttatcgtagccgtatcaatattggcaccggtgtgaaatttagcgagatcgataaaaatcaggtgcagctgagcaatctggaaagcagcaaaattgaagtgattctgaataacggcgtgatctacaatagcatgtatgaaaacttttcgaccagcttctggattcgcattccgaaatactttcgcaacatcaacaacgagtacaagattatcagctgtatgcagaataatagcggttgggaagttagcctgaatttcagcaatatgaacagcaaaatcatttggaccctgcaggataccgaaggtatcaaaaaaaccgttgtgtttcagtacacccagaacattaacatcagcgattacattaaccgctggatctttgtgaccattaccaataatcgtctgagcaacagcaagatctatattaacggtcgcctgattaacgaagagagcattagcgatctgggtaatattcatgccagcaacaacatcatgtttaaactggatggttgtcgtgatccgcatcgttatatttggatcaaatacttcaacctgtttgataaagaactgaacaaaaaagaaatcaaagacctgtatgataaccagagcaatagcggcattctgaaagatttttggggtgattatctgcagtatgacaaaccgtattacatgctgaatctgtacgatccgaacaaatatctggatgtgaataatgtgggtatccgtggctatatgtatctgaaaggtccgcgtggtcgtattgttaccaccaacatttatctgaatagcaccctgtatatgggcaccaaattcatcattaaaaagtatgccagcggcaacaaagataacattgtgcgtaataatgatcgcgtgtatatcaatgtggtggtgaagaataaagaatatcgtctggccaccaatgcaagccaggcaggcgttgaaaaaattctgagcgcagttgaaattccggatgttggtaatctgagccaggttgttgttatgaaaagcgaaaatgatcagggcattcgcaacaaatgcaaaatgaatctgcaggacaataacggcaacgatattggttttattggcttccaccagttcaacaacattgcaaaactggtggcgagcaattggtataatcgtcagattggtaaagcaagccgtacctttggttgtagctgggaatttattccggttgatgatggttggggtgaaagcagcctggaaaatctgtattttcagggtgcaagtcatcatcaccaccatcaccatcattaa

SEQ ID NO: 30 - protein, 446 aa, Artificial Sequence
  Polypeptide Sequence of rHC/FA (His-tagged)
MLKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 31 - DNA, 1347 bp, Artificial Sequence
  Nucleotide Sequence of rLC/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcgaaaatctgtattttcagggtgccagtcatcatcaccaccatcaccatcactga

SEQ ID NO: 32 - protein, 448 aa, Artificial Sequence
  Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSENLYFQGASHHHHHHHH

SEQ ID NO: 33 - DNA, 3888 bp, Artificial Sequence
  Nucleotide Sequence of rBoNT/F(0) (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacaccagctgatttatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaagatcaaggacaacagcatactggatatgcgctatgaaaacaacaagttcattgatatcagcggctatggtagcaacattagcattaatggtgatgtgtatatctacagcaccaaccgcaatcagtttggtatttatagcagcaaaccgagcgaagttaatattgcgcagaataacgatatcatctacaacggtcgctatcagaactttagcattagcttttgggttcgcattccgaaatactttaacaaggtgaacctgaacaacgagtacaccattattgattgcattcgcaataataacagcggctggaaaatcagcctgaactataacaaaattatctggaccctgcaggataccgcaggtaataatcagaaactggtgtttaactacacccagatgattagcatcagcgactatatcaacaaatggatctttgtgaccattaccaacaatcgtctgggtaacagccgcatttatatcaatggcaatctgatcgacgaaaaaagcatttcaaatctgggcgatattcacgtgagcgataacattctgttcaaaattgttggctgcaacgatacccgttatgttggtattcgttacttcaaagtgtttgatacggaactgggcaaaacggaaattgaaaccctgtatagtgatgaaccggatccgagcattctgaaagatttttggggtaattatctgctgtacaacaaacgctactatctgctgaacctgctgcgtaccgataaaagcattacacagaatagcaactttctgaacatcaatcagcagcgtggtgtttatcagaaaccgaacatttttagcaacacccgtctgtataccggtgtggaagttattattcgtaaaaacggtagcaccgatatcagcaacaccgataactttgtgcgtaaaaatgacctggcctatattaacgttgttgatcgtgatgttgagtatcgtctgtatgcggatattagcattgccaaaccggaaaagattatcaaactgatccgtaccagcaacagcaataattcactgggtcagattatcgtgatggacagcattggtaacaattgcaccatgaatttccagaacaataacggtggtaatattggcctgctgggctttcatagcaataatctggttgcaagcagctggtattacaacaacatccgtaaaaataccagcagtaatggttgcttttggagctttatcagtaaagaacatggctggcaagaaaacgagaacctgtattttcagggtgcaagtcatcatcaccatcaccaccatcattaa

SEQ ID NO: 34 - protein, 1295 aa, Artificial Sequence
  Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 35 - DNA, 2649 bp, Artificial Sequence
  Nucleotide Sequence of rLHN/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaaagaaaacctgtattttcagggtgcaagccatcatcaccaccatcaccatcattaa

SEQ ID NO: 36 - protein, 882 aa, Artificial Sequence
  Polypeptide Sequence of rLHN/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKENLYFQGASHHHHHHHH

SEQ ID NO: 37 - DNA, 1296 bp, Artificial Sequence
  Nucleotide Sequence of rHC/F (His-tagged)
atgatcaaggataacagcattctggatatgcgctatgagaacaacaaattcattgatattagcggctatggcagcaacattagcattaatggtgatgtgtatatctacagcaccaaccgtaatcagtttggcatttatagcagcaaaccgagcgaagttaatattgcccagaacaacgatatcatctataacggtcgctatcagaacttcagcattagcttttgggttcgcattccgaaatacttcaataaggtgaacctgaacaacgagtataccatcattgattgcattcgcaataataacagcggctggaaaattagcctgaactacaacaaaattatctggaccctgcaggataccgcaggtaataatcagaaactggtgtttaactacacccagatgattagcatcagcgactatatcaacaaatggatctttgtgaccattaccaataatcgcctgggtaatagccgcatttatatcaatggtaacctgatcgatgagaaaagcattagcaatctgggtgatattcatgtgagcgataacatcctgtttaaaatcgtgggttgtaacgatacccgttatgttggtattcgctacttcaaagtgtttgataccgaactgggtaaaaccgaaattgaaaccctgtatagtgatgaaccggatccgagcattctgaaagatttttggggtaattatctgctgtacaacaaacgctactatctgctgaatctgctgcgtaccgataaatcaattacccagaatagcaacttcctgaacattaatcagcagcgtggtgtttatcagaaaccgaacatttttagcaacacccgtctgtataccggtgtggaagttattattcgtaaaaatggcagcaccgatatcagcaacaccgataactttgttcgcaaaaatgatctggcgtatatcaacgttgttgatcgtgatgttgaatatcgtctgtatgccgatattagcattgccaaaccggaaaaaatcatcaaactgatccgtaccagcaacagcaataattcactgggtcagattattgtgatggatagcattggtaataactgcaccatgaactttcagaacaataacggtggtaatattggtctgctgggctttcatagtaataatctggttgcaagcagctggtattataacaacatccgtaaaaataccagcagcaatggttgcttttggagctttattagcaaagaacatggctggcaagaaaacgagaatctgtattttcagggtgcaagtcatcatcaccaccatcaccatcattaa

SEQ ID NO: 38 - protein, 431 aa, Artificial Sequence
  Polypeptide Sequence of rHC/F (His-tagged)
MIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 39 - DNA, 1347 bp, Artificial Sequence
  Nucleotide Sequence of rLC/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgagaacctgtattttcagggtgcaagtcatcatcaccatcaccaccatcattaa

SEQ ID NO: 40 - protein, 448 aa, Artificial Sequence
  Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSENLYFQGASHHHHHHHH

SEQ ID NO: 41 - DNA, 1317 bp, Artificial Sequence
  Nucleotide Sequence of Cationic rHC/A (His-tagged)
atgatcatcaacaccagcattctgaacctgcgttatgaaagcaaacatctgattgatctgagccgttatgccagcaaaatcaatataggcagcaaggttaacttcgacccgattgacaaaaatcagatacagctgtttaatctggaaagcagcaaaattgaggtgatcctgaaaaaagcgatcgtgtataatagcatgtacgagaatttttcgaccagcttttggattcgcatcccgaaatactttaacaagattagcctgaacaacgagtataccatcattaactgcatggaaaacaatagcggttggaaagtcagcctgaattatggcgaaattatctggaccctgcaggataccaaagaaatcaaacagcgtgtggtgttcaaatacagccagatgattaatatcagcgactatatcaaccgctggatttttgtgaccattaccaataatcggctgaacaagagcaagatctatattaacggtcgtctgattgaccagaaaccgattagtaatctgggtaatattcatgcgagcaacaaaatcatgtttaaactggatggttgccgtgatacccatcgttatatttggatcaaatacttcaacctgttcgataaagagttgaacgaaaaagaaattaaagacctgtacgataaccagagcaatagcggcatactgaaagatttttggggagattatctgcagtatgacaaaccgtattatatgctgaatctgtacgacccgaataaatacgtggatgttaataatgtgggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaacgataaaggcatcaccaacaaatgcaagatgaatctgcaggacaataacggcaatgatattggcttcattggctttcaccagtttaacaacattgcaaaactggttgcgagcaattggtataatcgtcagattgaacgtagcagtcgtaccctgggttgtagctgggaatttatccctgtggatgatggttggggtgaacgtccgctgaagcttgcggccgcactcgagcaccaccaccaccaccactga

SEQ ID NO: 42 - protein, 438 aa, Artificial Sequence
  Polypeptide Sequence of Cationic rHC/A (His-tagged)
MIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPLKLAAALEHHHHHH

SEQ ID NO: 43 - DNA, 1341 bp, Artificial Sequence
  Nucleotide Sequence of rHC/AB (His-tagged)
atgattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaaaagcttgcggccgcactcgagcaccaccaccaccaccactga

SEQ ID NO: 44 - protein, 446 aa, Artificial Sequence
  Polypeptide Sequence of rHC/AB (His-tagged)
MILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEKLAAALEHHHHHH

SEQ ID NO: 45 - DNA, 1306 bp, Artificial Sequence
  Nucleotide Sequence of rHC/A Variant Y1117V H1253K (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttcaaacagttcaacaatattgctaaactggtagcgagcaattggtacaatcgtcagattgagcgcagcagccgtactttgggctgtagctgggagtttatcccggtcgatgatggttggggcgaacgtccgctgcaccatcaccatcaccatcaccatcaccatt

SEQ ID NO: 46 - protein, 435 aa, Artificial Sequence
  Polypeptide Sequence of rHC/A Variant Y1117V H1253K (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFKQFNNIAKLVASNWYNRQIERSSRTLGCSWEFIPVDDGWGERPLHHHHHHHHHH

SEQ ID NO: 47 - DNA, 1308 bp, Artificial Sequence
  Nucleotide Sequence of rHC/A Variant Y1117V F1252Y H1253K L1278F (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtacttttggctgtagctgggagtttatcccggtcgatgatggttggggcgaacgtccgctgcaccatcaccatcaccatcaccatcaccattaa

SEQ ID NO: 48 - protein, 435 aa, Artificial Sequence
  Polypeptide Sequence of rHC/A Variant Y1117V F1252Y H1253K L1278F (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTFGCSWEFIPVDDGWGERPLHHHHHHHHHH

SEQ ID NO: 49 - DNA, 1293 bp, Artificial Sequence
  Nucleotide Sequence of rHC/A Variant Y1117V F1252Y H1253K L1278H (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtactcatggctgtagctgggagtttatcccggtcgatgatggttggggcgaacgtccgctgcaccatcaccatcaccat

SEQ ID NO: 50 - protein, 431 aa, Artificial Sequence
  Polypeptide Sequence of rHC/A Variant Y1117V F1252Y H1253K L1278H (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTHGCSWEFIPVDDGWGERPLHHHHHH

SEQ ID NO: 51 - protein, 1296 aa, Clostridium botulinum [sequence omitted]

SEQ ID NO: 52 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 53 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 54 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 55 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 56 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 57 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 58 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 59 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 60 - DNA, 3888 bp, Artificial Sequence
  Nucleotide Sequence of mrBoNT/A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 61 - protein, 1296 aa, Artificial Sequence
  Polypeptide Sequence of mrBoNT/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 62 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 63 - protein, 1304 aa, Artificial Sequence
  Polypeptide Sequence of mrBoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 64 - protein, 1304 aa, Artificial Sequence
  Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 65 - protein, 1296 aa, Artificial Sequence
  Polypeptide Sequence of mrBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 66 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XDXXXLL

SEQ ID NO: 67 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLL

SEQ ID NO: 68 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXIL

SEQ ID NO: 69 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLM

SEQ ID NO: 70 - protein, 4 aa, Artificial Sequence
  Tyrosine-Based Motif
  SITE (2)..(3): Xaa can be any naturally occurring amino acid
  VARIANT (4)..(4): Xaa is a hydrophobic amino acid
YXXX

SEQ ID NO: 71 - protein, 6 aa, Clostridium botulinum
NIINTS

SEQ ID NO: 72 - protein, 6 aa, Clostridium botulinum
NIVNTS

SEQ ID NO: 73 - protein, 6 aa, Clostridium botulinum
NITNAS

SEQ ID NO: 74 - protein, 6 aa, Clostridium botulinum
NITNTS

SEQ ID NO: 75 - protein, 6 aa, Clostridium botulinum
EILNNI

SEQ ID NO: 76 - protein, 6 aa, Clostridium botulinum
DILNNI